(19) United States
(12) Patent Application Publication
(10) Pub. No.: US 2021/0166781 A1
(43) Pub. Date: Jun. 3, 2021

(54) METHODS AND SYSTEMS FOR DIAGNOSING FROM WHOLE GENOME SEQUENCING DATA

(71) Applicant: Illumina, Inc., San Diego, CA (US)

(72) Inventors: Michael A. Eberle, San Diego, CA (US); Xiao Chen, San Diego, CA (US)

(21) Appl. No.: 17/003,856

(22) Filed: Aug. 26, 2020

Related U.S. Application Data
(60) Provisional application No. 62/896,548, filed on Sep. 5, 2019.
(60) Provisional application No. 62/908,555, filed on Sep. 30, 2019.
(60) Provisional application No. 63/006,651, filed on Apr. 7, 2020.

(51) Int. Cl. G16B 20/10; G16B 30/10; G16B 20/20; G16B 5/20

(52) U.S. Cl. CPC G16B 20/10; G16B 5/20; G16B 20/20; G16B 30/10

(57) ABSTRACT

Disclosed herein include systems, devices, computer readable media, and methods for paralog genotyping, such as determining a copy number of survival of motor neuron 1 gene and genotyping cytochrome P450 family 2 subfamily D member 6 gene using a Gaussian mixture model comprising a plurality of Gaussians each representing a different integer copy number.

### CROSS-REFERENCE TO RELATED APPLICATIONS

This application claims the benefit of priority to U.S. Provisional Patent Application No. 62/896,548, filed on Sep. 5, 2019, U.S. Provisional Patent Application No. 62/908,555, filed on Sep. 30, 2019, and U.S. Provisional Patent Application No. 63/006,651, filed on Apr. 7, 2020. The content of each of the related applications is incorporated herein by reference herein in its entirety.

### BACKGROUND

### Field

This disclosure relates to relates generally to the field of paralog genotyping, and more particularly to paralog genotyping using sequencing data.

### Background

Genotyping is challenging. For example, spinal muscular atrophy is caused by loss of the functional survival of motor neuron 1 (SMN1) gene but retention of the paralogous SMN2 gene. Due to the near identical sequences of SMN1 and its paralog SMN2, analysis of this region has been challenging. As another example, CYP2D6 is involved in the metabolism of 25% of all drugs. Genotyping CYP2D6 is challenging due to its high polymorphism, the presence of common structural variants (SVs), and high sequence similarity with the gene's pseudogene paralog CYP2D7.

### SUMMARY

Disclosed herein include methods for determining a copy number of survival of motor neuron 1 (SMN1) gene. In some embodiments, a method for determining a copy number of SMN1 gene is under control of a processor (such as a hardware processor or a virtual processor) and comprises: receiving sequence data comprising a plurality of sequence reads obtained from a sample of a subject aligned to SMN1 gene or survival of motor neuron 2 (SMN2) gene. The method can comprise: determining (i) a first number of sequence reads of the plurality of sequence reads aligned to a first SMN1 or SMN2 region comprising at least one of exon 1 to exon 6 of the SMN1 gene or the SMN2 gene, respectively, and (ii) a second number of sequence reads of the plurality of sequence reads aligned to a second SMN1 or SMN2 region comprising at least one of exon 7 and exon 8 of the SMN1 gene or the SMN2 gene, respectively. The method can comprise: determining (i) a first normalized number of the sequence reads aligned to the first SMN1 or SMN2 region and (ii) a second normalized number of the sequence reads aligned to the second SMN1 or SMN2 region using (i) a length of the first SMN1 or SMN2 region and (ii) a length of the second SMN1 or SMN2 region, respectively. The method can comprise: determining (i) a copy number of total survival of motor neuron (SMN) genes, each being an intact SMN1 gene, an intact SMN2 gene, a truncated SMN1 gene, or a truncated SMN2 gene, and (ii) a copy number of any intact SMN genes, each being the intact SMN1 gene or the intact SMN2 gene, using a Gaussian mixture model comprising a plurality of Gaussians each representing a different integer copy number, given (i) the first normalized number of the sequence reads aligned to the first SMN1 or SMN2 region and (ii) the second normalized number of the sequence reads aligned to the second SMN1 or SMN2 region, respectively. The method can comprise: for one of a plurality of SMN1 gene-specific bases associated with the intact SMN1 gene, determining a most likely combination, of a plurality of possible combinations each comprising a possible copy number of the SMN1 gene and a possible copy number of the SMN2 gene summed to the copy number of any intact SMN genes determined, given (a) a number of sequence reads of the plurality of sequence reads with bases that support the SMN1 gene-specific base and (b) a number of sequence reads of the plurality of sequence reads with bases that support a SMN2 gene-specific base of the SMN2 gene corresponding to the SMN1 gene-specific base. The method can comprise: determining a copy number of the SMN1 gene using the most likely combination of the possible copy number of the SMN1 gene and the possible copy number of the SMN2 gene determined for the SMN1 gene-specific base.

In some embodiments, the sequencing data comprises whole genome sequencing (WGS) data or short-read WGS data. In some embodiments, the subject is a fetal subject, a neonatal subject, a pediatric subject, an adolescent subject, or an adult subject. The sample can comprise cells or cell-free DNA. The sample can comprise fetal cells or cell-free fetal DNA.

In some embodiments, a sequence read of the plurality of sequence reads is aligned to the first SMN1 or SMN2 region or the second SMN1 or SMN2 region with an alignment quality score of about zero. The first SMN1 or SMN2 region can comprise the exon 1 to the exon 6 of the SMN1 gene or the SMN2 gene, respectively, and is about 22.2 kb in length. The second SMN1 or SMN2 region can comprise the exon 7 and the exon 8 of the SMN1 gene or the SMN2 gene, respectively, and is about 6 kb in length.

In some embodiments, determining (i) the first normalized number of the sequence reads aligned to the first SMN1 or SMN2 region and (ii) the second normalized number of the sequence reads aligned to the second region comprises: determining (i) the first normalized number of the sequence reads aligned to the first SMN1 or SMN2 region and (ii) the second normalized number of the sequence reads aligned to the second SMN1 or SMN2 region using (i) the length of the first SMN1 or SMN2 region and (ii) the length of the second SMN1 or SMN2 region, respectively, and (iii) a depth of sequence reads of a region of a genome of the subject other than genetic loci comprising the SMN1 gene and the SMN2 gene in the sequence data. Determining (i) the first normalized number of the sequence reads aligned to the first SMN1 or SMN2 region and (ii) the second normalized number of the sequence reads aligned to the second SMN1 or SMN2 region can comprise: determining (i) a first SMN1 or SMN2 region-length normalized number of the sequence reads aligned to the first SMN1 or SMN2 region and (ii) a second SMN1 or SMN2 region-length normalized number of the sequence reads aligned to the second SMN1 or SMN2 region using (i) the length of the first SMN1 or SMN2 region and (ii) the length of the second SMN1 or SMN2 region, respectively. Determining (i) the first normalized number of the sequence reads aligned to the first SMN1 or SMN2 region and (ii) the second normalized number of the sequence reads aligned to the second SMN1 or SMN2 region can comprise: determining (i) a first normalized depth of the sequence reads aligned to the first region SMN1 or SMN2 and (ii) a second normalized depth of the sequence reads aligned to the second SMN1 or SMN2 region from (i) the first SMN1 or SMN2 region-length normalized number and (ii) the second SMN1 or SMN2 region-length normalized number, respectively, using the depth of the sequence reads of the region of the genome of the subject other than genetic loci comprising the SMN1 gene and the SMN2 gene, the first normalized number of the sequence reads aligned to the first SMN1 or SMN2 region and the second normalized number of the sequence reads aligned to the second SMN1 or SMN2 region being the first normalized depth and the second normalized depth, respectively.

In some embodiments, determining (i) the first normalized number of the sequence reads aligned to the first SMN1 or SMN2 region and (ii) the second normalized number of the sequence reads aligned to the second region comprises: determining (i) the first normalized number of the sequence reads aligned to the first SMN1 or SMN2 region and (ii) the second normalized number of the sequence reads aligned to the second SMN1 or SMN2 region using (i) a GC content of the first SMN1 or SMN2 region and (ii) a GC content of the second SMN1 or SMN2 region, respectively, and (iii) a depth of sequence reads of a region of a genome of the subject other than genetic loci comprising the SMN1 gene and the SMN2 gene in the sequence data, and (iv) a GC content of the region of the genome.

In some embodiments, the depth of the region comprises an average depth or a median depth of the sequence reads of the region of the genome of the subject other than the genetic loci comprising the SMN1 gene and the SMN2 gene in the sequencing data. The region can comprise about 3000 pre-selected regions of about 2 kb in length each across the genome of the subject. In some embodiments, (i) the first normalized number of the sequence reads aligned to the first SMN1 or SMN2 region and/or (ii) the second normalized number of the sequence reads aligned to the second SMN1 or SMN2 region is about 30 to about 40.

In some embodiments, the Gaussian mixture model comprises a one-dimensional Gaussian mixture model. The plurality of Gaussians of the Gaussian mixture model can represent integer copy numbers 0 to 10. A mean of each of the plurality of Gaussians can be the integer copy number represented by the Gaussian.

In some embodiments, determining (i) the copy number of the total SMN genes and (ii) the copy number of any intact SMN genes comprises determining (i) the copy number of the total SMN genes and (ii) the copy number of any intact SMN genes using the Gaussian mixture model, and a first predetermined posterior probability threshold, given (i) the first normalized number of the sequence reads aligned to the first SMN1 or SMN2 region and (ii) the second normalized number of the sequence reads aligned to the second SMN1 or SMN2 region, respectively. The first predetermined posterior probability threshold can be 0.95.

In some embodiments, the method comprises: determining a copy number of truncated SMN genes using (i) the copy number of the total SMN genes determined and (ii) the copy number of the intact SMN genes determined. The copy number of the truncated SMN genes can be a difference of (i) the copy number of the total SMN genes determined and (ii) the copy number of the intact SMN genes determined.

In some embodiments, the SMN1 gene-specific base is a splicing enhancer. The SMN1 gene-specific base can be a base at c.840 of the SMN1 gene. In some embodiments, the most likely combination of the possible copy number of the SMN1 gene and the possible copy number of the SMN2 gene is associated with a highest posterior probability, relative to other combinations of the plurality of combinations given (a) the number of sequence reads of the plurality of sequence reads with bases that support the SMN1 gene-specific base and (b) the number of sequence reads of the plurality of sequence reads with bases that support the corresponding SMN2 gene-specific base.

In some embodiments, determining the most likely combination of the possible copy number of the SMN1 gene and the possible combination of the SMN2 gene comprises: determining the most likely combination, of the plurality of possible combinations each comprising a possible copy number of the SMN1 gene and a possible copy number of the SMN2 gene summed to the copy number of any intact SMN genes determined, given a ratio of (a) a number of sequence reads of the plurality of sequence reads with bases that support the SMN1 gene-specific base and (b) a number of sequence reads of the plurality of sequence reads with bases that support the SMN2 gene-specific base of the SMN2 gene corresponding to the SMN1 gene-specific base. Determining the most likely combination of the possible copy number of the SMN1 gene and the possible combination of the SMN2 gene can comprise: determining (a) a number of sequence reads of the plurality of sequence reads with bases that support the SMN1 gene-specific base and (b) a number of sequence reads of the plurality of sequence reads with bases that support the SMN2 gene-specific base of the SMN2 gene corresponding to the SMN1 gene-specific base; determining the ratio of (a) a number of sequence reads of the plurality of sequence reads with bases that support the SMN1 gene-specific base and (b) a number of sequence reads of the plurality of sequence reads with bases that support the SMN2 gene-specific base of the SMN2 gene corresponding to the SMN1 gene-specific base; and determining the most likely combination, of the plurality of possible combinations each comprising a possible copy number of the SMN1 gene and a possible copy number of the SMN2 gene summed to the copy number of any intact SMN genes determined based on the ratio of (a) a number of sequence reads of the plurality of sequence reads with bases that support the SMN1 gene-specific base and (b) a number of sequence reads of the plurality of sequence reads with bases that support the SMN2 gene-specific base of the SMN2 gene corresponding to the SMN1 gene-specific base.

In some embodiments, determining the most likely combination of the possible copy number of the SMN1 gene and the possible combination of the SMN2 gene comprises: for each of the plurality of SMN1 gene-specific bases, determining a most likely combination, of a plurality of possible combinations each comprising a possible copy number of the SMN1 gene and a possible copy number of the SMN2 gene summed to the copy number of any intact SMN genes determined, a being associated with a highest posterior probability given (a) a number of sequence reads of the plurality of sequence reads with bases that support the SMN1 gene-specific base and (b) a number of sequence reads of the plurality of sequence reads with bases that support a SMN2 gene-specific base of the SMN2 gene corresponding to the SMN1 gene-specific base. Determining the copy number of the SMN1 gene can comprise: determining the copy number of the SMN1 gene based on the possible copy number of the SMN1 gene of the most likely combination of the possible copy number of the SMN1 gene and the possible copy number of the SMN2 gene determined for each of the plurality of SMN1 gene-specific bases.

In some embodiments, the SMN1 gene-specific base has a concordance with each of the plurality of SMN1 gene-specific bases other than the SMN1 gene-specific base above a predetermined concordance threshold. The concordance threshold can be 97%. The plurality of SMN1 gene-specific bases can comprise 8 SMN1 gene-specific bases. Each of the plurality of SMN1 gene-specific bases can be on intron 6, the exon 7, intron 7, or the exon 8 of the SMN1 gene. The plurality of SMN1 gene-specific bases if the subject is of a first race, the plurality of SMN1 gene-specific bases if the subject is of a second race, and the plurality of SMN1 gene-specific bases if the subject is of an unknown race can be different. A race of the subject can be unknown, and the plurality of SMN1 gene-specific bases can be race non-specific. A race of the subject can be known, and the plurality of SMN1 gene-specific bases can specific to the race of the subject. In some embodiments, the method comprises: receiving race information of the subject. The method can comprise: selecting the plurality of SMN1 gene-specific bases from pluralities of SMN1 gene-specific bases based on the race information received.

In some embodiments, determining the copy number of the SMN1 gene comprises: determining the copy number of the SMN1 gene and a copy number of the SMN2 gene using the most likely combination of the possible copy number of the SMN1 gene and the possible copy number of the SMN2 gene determined for each of the plurality of SMN1 gene-specific bases. Determining the copy number can comprise: determining the copy number of the SMN1 gene using the most likely combination of the possible copy number of the SMN1 gene and the possible copy number of the SMN2 gene determined for the SMN1 gene-specific base and a second predetermined posterior probability threshold for the combination of the possible copy number of SMN1 gene and the possible copy number of the SMN2 gene. The second predetermined posterior probability threshold can be 0.6 or 0.8.

In some embodiments a majority of the possible copy numbers of the SMN1 gene determined agree. The copy number of the SMN1 gene determined can be the agreed possible copy number of the SMN1 gene. The method can comprise: determining a possible combination comprising a possible copy number of the SMN1 gene and a possible copy number of the SMN2 gene summed to the copy number of any intact SMN genes determined given (a) a number of sequence reads of the plurality of sequence reads with bases that support any of the plurality of SMN1 gene-specific bases and (b) a number of sequence reads of the plurality of sequence reads with bases that support any of the plurality of corresponding SMN2 gene-specific bases. The method can comprise: determining the possible copy number of the possible combination is the agreed possible copy number of the SMN1 gene.

In some embodiments, determining the copy number of the SMN1 gene comprises: determining the copy number of the SMN1 gene to be zero, one, or more than one. In some embodiments, the method comprises: determining a spinal muscular atrophy (SMA) status of the subject based on the copy number of the SMN1 gene. The SMA status of the subject can comprise SMA, SMA carrier/not SMA, and not SMA carrier. In some embodiments, the method comprises: determining subject is a silent SMA carrier using a number of sequence reads of the plurality of sequence reads aligned to g.27134 of the SMN1 gene and the bases of the sequence reads aligned to the g.27134 of the SMN1 gene.

In some embodiments, the method comprises: determining a treatment recommendation for the subject based on the copy number of the SMN1 gene determined. The treatment recommendation can comprise administering Nusinersen and/or Zolgensma to the subject.

Disclosed herein includes methods for genotyping cytochrome P450 family 2 subfamily D member 6 (CYP2D6) gene. In some embodiments, a method for genotyping CYP2D6 gene is under control of a processor (such as a hardware processor or a virtual processor) and comprises: receiving sequence data comprising a plurality of sequence reads obtained from a sample of a subject aligned to CYP2D6 gene or cytochrome P450 Family 2 Subfamily D Member 7 (CYP2D7) gene. The method can comprise: determining (i) a first number of sequence reads of the plurality of sequence reads aligned to the CYP2D6 gene or the CYP2D7 gene. The method can comprise: determining (i) a first normalized number of the sequence reads aligned to the CYP2D6 gene or the CYP2D7 gene using (i) a length of the CYP2D6 gene or the CYP2D7 gene, respectively. The method can comprise: determining (i) a total copy number of the CYP2D6 gene and the CYP2D7 gene using a Gaussian mixture model comprising a plurality of Gaussians each representing a different integer copy number, given (i) the first normalized number of the sequence reads aligned to the CYP2D6 gene or the CYP2D7 gene. The method can comprise: for one of a plurality of CYP2D6 gene-specific bases, determining a most likely combination, of a plurality of possible combinations each comprising a possible copy number of the CYP2D6 gene and a possible copy number of the CYP2D7 gene summed to the total copy number of the CYP2D6 gene and the CYP2D7 gene determined, given (a) a number of sequence reads of the plurality of sequence reads with bases that support the CYP2D6 gene-specific base and (b) a number of sequence reads of the plurality of sequence reads with bases that support a CYP2D7 gene-specific base of corresponding to the CYP2D6 gene-specific base. The method can comprise: determining an allele of the CYP2D6 gene the subject has using the most likely combination of the possible copy number of the CYP2D6 gene and the possible copy number of the CYP2D7 gene determined for the CYP2D6 gene-specific base.

In some embodiments, the sequencing data comprises whole genome sequencing (WGS) data or short-read WGS data. The subject can be a fetal subject, a neonatal subject, a pediatric subject, an adolescent subject, or an adult subject. The sample can comprise cells or cell-free DNA. The sample can comprise cells or cell-free DNA.

In some embodiments, a sequence read of the plurality of sequence reads is aligned to the CYP2D6 gene or the CYP2D7 gene with an alignment quality score of about zero. In some embodiments, determining (i) the first number of sequence reads of the plurality of sequence reads aligned to the CYP2D6 gene or the CYP2D7 gene comprises: determining (i) the first number of sequence reads of the plurality of sequence reads aligned to at least one exon or intron of the CYP2D6 gene or at least one of exon or intron of the CYP2D7 gene.

In some embodiments, determining (i) the first normalized number of the sequence reads aligned to the CYP2D6 gene or the CYP2D7 gene comprises: determining (i) the first normalized number of the sequence reads aligned to the CYP2D6 gene or the CYP2D7 gene using (i) the length of the CYP2D6 gene or the CYP2D7 gene, respectively, and (iii) a depth of sequence reads of a region of a genome of the subject other than genetic loci comprising the CYP2D6 gene and the CYP2D7 gene in the sequence data. Determining (i) the first normalized number of the sequence reads aligned to the CYP2D6 gene or the CYP2D7 gene and (ii) the second normalized number of the sequence reads aligned to the second region can comprises: determining (i) a first CYP2D6 gene or the CYP2D7 gene-length normalized number of the sequence reads aligned to the CYP2D6 gene or the CYP2D7 gene using (i) the length of the CYP2D6 gene or the CYP2D7 gene, respectively. Determining (i) the first normalized number of the sequence reads aligned to the CYP2D6 gene or the CYP2D7 gene and (ii) the second normalized number of the sequence reads aligned to the second region can comprises: determining (i) a first normalized depth of the sequence reads aligned to the CYP2D6 gene or the CYP2D7 gene from (i) the CYP2D6 gene or the CYP2D7 gene-length normalized number using the depth of the sequence reads of the region of the genome of the subject other than genetic loci comprising the CYP2D6 gene and the CYP2D7, the first normalized depth of the sequence reads aligned to the CYP2D6 gene or the CYP2D7 gene being the first normalized number of the sequence reads aligned to the CYP2D6 gene or CYP2D7 gene, respectively.

In some embodiments, determining (i) the first normalized number of the sequence reads aligned to the CYP2D6 gene or the CYP2D7 gene comprises: determining (i) the first normalized number of the sequence reads aligned to the CYP2D6 gene or the CYP2D7 gene using (i) a GC content of the CYP2D6 gene or the CYP2D7 gene and (iii) a depth of sequence reads of a region of a genome of the subject other than genetic loci comprising the CYP2D6 gene and the CYP2D7 gene in the sequence data, and (iv) a GC content of the region of the genome. The depth of the region can comprise an average depth or a median depth of the sequence reads of the region of the genome of the subject other than the genetic loci comprising the CYP2D6 gene and the CYP2D7 gene in the sequencing data. The region can comprise about 3000 pre-selected regions of about 2 kb in length each across the genome of the subject. In some embodiments, (i) the first normalized number of the sequence reads aligned to the CYP2D6 gene or the CYP2D7 gene and/or (ii) the second normalized number of the sequence reads aligned to the second region is about 30 to about 40.

In some embodiments, the Gaussian mixture model comprises a one-dimensional Gaussian mixture model. The plurality of Gaussians of the Gaussian mixture model can represent integer copy numbers 0 to 10. A mean of each of the plurality of Gaussians can be the integer copy number represented by the Gaussian.

In some embodiments, determining (i) the total copy number of the CYP2D6 gene and the CYP2D7 gene comprises determining (i) the total copy number of the CYP2D6 gene and the CYP2D7 gene using the Gaussian mixture model and a first predetermined posterior probability threshold, given (i) the first normalized number of the sequence reads aligned to the CYP2D6 gene or the CYP2D7 gene. The first predetermined posterior probability threshold can be 0.95.

In some embodiments, the most likely combination of the possible copy number of the CYP2D6 gene and the possible copy number of the CYP2D7 gene is associated with a highest posterior probability, relative to other combinations of the plurality of combinations given (a) the number of sequence reads of the plurality of sequence reads with bases that support the CYP2D6 gene-specific base and (b) the number of sequence reads of the plurality of sequence reads with bases that support the corresponding CYP2D7 gene-specific base.

In some embodiments, determining the most likely combination comprising a possible copy number of the CYP2D6 gene and the possible copy number CYP2D7 gene comprises: determining the most likely combination, of the plurality of possible combinations each comprising a possible copy number of the CYP2D6 gene and a possible copy number of the CYP2D7 gene summed to the total copy number of the CYP2D6 gene and the CYP2D7 gene determined, given a ratio of (a) the number of sequence reads of the plurality of sequence reads with bases that support the CYP2D6 gene-specific base and (b) the number of sequence reads of the plurality of sequence reads with bases that support a CYP2D7 gene-specific base of corresponding to the CYP2D6 gene-specific base. Determining the most likely combination comprising a possible copy number of the CYP2D6 gene and a possible copy number can comprise: determining (a) the number of sequence reads of the plurality of sequence reads with bases that support the CYP2D6 gene-specific base and (b) the number of sequence reads of the plurality of sequence reads with bases that support a CYP2D7 gene-specific base of corresponding to the CYP2D6 gene-specific base; determining a ratio of (a) the number of sequence reads of the plurality of sequence reads with bases that support the CYP2D6 gene-specific base and (b) the number of sequence reads of the plurality of sequence reads with bases that support a CYP2D7 gene-specific base of corresponding to the CYP2D6 gene-specific base; and determining the most likely combination, of the plurality of possible combinations each comprising a possible copy number of the CYP2D6 gene and a possible copy number of the CYP2D7 gene summed to the total copy number of the CYP2D6 gene and the CYP2D7 gene determined, given the ratio (a) a number of sequence reads of the plurality of sequence reads with bases that support the CYP2D6 gene-specific base and (b) a number of sequence reads of the plurality of sequence reads with bases that support a CYP2D7 gene-specific base of corresponding to the CYP2D6 gene-specific base.

In some embodiments, determining the allele of the CYP2D6 gene the subject has comprises: determining one or more structural variants of the CYP2D6 gene the subject has using the most likely combination of the possible copy number of the CYP2D6 gene and the possible copy number of the CYP2D7 gene determined for the CYP2D6 gene-specific base. In some embodiments, determining the most likely combination of the possible copy number of the CYP2D6 gene and the possible copy number of the CYP2D7 gene comprises: for each of the plurality of CYP2D6 gene-specific bases, determining a most likely combination, of a plurality of possible combinations each comprising a possible copy number of the CYP2D6 gene and a possible copy number of the CYP2D7 gene summed to the total copy number the CYP2D6 gene and the CYP2D7 gene determined, associated with a highest posterior probability given (a) a number of sequence reads of the plurality of sequence reads with bases that support the CYP2D6 gene-specific base and (b) a number of sequence reads of the plurality of sequence reads with bases that support a CYP2D7 gene-specific base of the CYP2D7 gene corresponding to the CYP2D6 gene-specific base. Determining the one or more structural variants of the CYP2D6 gene the subject has can comprise determining the one or more structural variants using the most likely combination of the possible copy number of the CYP2D6 gene and the possible copy number of the CYP2D7 gene determined for each of the plurality of CYP2D6 gene-specific bases. In some embodiments, determining the one or more structural variants of the CYP2D6 gene the subject has comprises: determining one or more structural variants of the CYP2D6 gene the subject has based on the copy numbers of the CYP2D6 gene of the most likely combinations determined for two or more of the plurality of CYP2D6 gene-specific bases that are different and the positions of the two or more CYP2D6 gene-specific bases.

In some embodiments, the CYP2D6 gene-specific base has a concordance with each of the plurality of CYP2D6 gene-specific bases other than the CYP2D6 gene-specific base above a predetermined concordance threshold. The concordance threshold can be 97%. The plurality of CYP2D6 gene-specific bases can comprise 118 CYP2D6 gene-specific bases. The plurality of CYP2D6 gene-specific bases if the subject is of a first race, the plurality of CYP2D6 gene-specific bases if the subject is of a second race, and the plurality of CYP2D6 gene-specific bases if the subject is of an unknown race can be different. A race of the subject can be unknown, and the plurality of CYP2D6 gene-specific bases can be race non-specific. A race of the subject can be known, and the plurality of CYP2D6 gene-specific bases can be specific to the race of the subject. In some embodiments, the method comprises: receiving race information of the subject. The method can comprise: selecting the plurality of CYP2D6 gene-specific bases from pluralities of CYP2D6 gene-specific bases based on the race information received.

In some embodiments, the method comprises: determining (ii) a second number of sequence reads of the plurality of sequence reads aligned to a spacer region between the CYP2D7 gene and a repetitive element REP7 downstream of the CYP2D7 gene. The method can comprise: determining (ii) a second normalized number of the sequence reads aligned to the spacer region using (ii) a length of the spacer region. The method can comprise: determining (ii) a copy number of the spacer region using the Gaussian mixture model given (ii) the second normalized number of the sequence reads aligned to the spacer region. Determining the one or more structural variants of the CYP2D6 gene the subject has can comprise: determining the one or more structural variants of the CYP2D6 gene the subject has using the most likely combination of the possible copy number of the CYP2D6 gene and the possible copy number of the CYP2D7 gene determined for the CYP2D6 gene-specific base and the copy number of the spacer region. The one or more structural variants can comprise a CYP2D6/CYP2D7 fusion allele with the spacer region and the repetitive element REP7 downstream of the CYP2D6/CYP2D7 fusion allele.

In some embodiments, the method comprises: determining one or more small variants of the CYP2D6 gene the subject has using the sequence data received. In some embodiments, determining the one or more small variants of the CYP2D6 gene the subject has comprises: for a small variant position of the CYP2D6 gene associated with a small variant allele of the CYP2D6 gene, determining a most likely combination of a possible copy number of the small variant allele of the CYP2D6 gene at the small variant position and a possible copy number of a reference allele of the CYP2D6 gene summed to a copy number of the CYP2D6 gene at the small variant position, given (a) a number of sequence reads with bases supporting the small variant allele of the CYP2D6 gene at the small variant position and (b) a number of sequence reads with bases supporting the reference allele of the CYP2D6 gene at the small variant position, the possible copy number of the small variant allele of the CYP2D6 gene of the most likely combination at the small variant position indicates the one or more small variants of the CYP2D6 gene. In some embodiments, determining the one or more small variants of the CYP2D6 gene the subject has comprises: for each of a plurality of small variant positions of the CYP2D6 gene, the small variant position being associated with a small variant allele of the CYP2D6 gene, determining a most likely combination of a possible copy number of the small variant allele of the CYP2D6 gene at the small variant position and a possible copy number of a reference allele of the CYP2D6 gene at the small variant position summed to a copy number of the CYP2D6 gene at the small variant position, given (a) a number of sequence reads with bases supporting the small variant allele of the CYP2D6 gene at the small variant position and (b) a number of sequence reads with bases supporting the reference allele of the CYP2D6 gene at the small variant position, the possible copy numbers of the small variant alleles of the CYP2D6 gene of the most likely combinations at the plurality of small variant positions indicate the one or more small variants of the CYP2D6 gene.

In some embodiments, the method comprises: for a small variant position of the CYP2D6 gene associated with a small variant allele of the CYP2D6 gene, determining a most likely combination of a possible copy number of the small variant allele of the CYP2D6 gene at the small variant position and a possible copy number of a reference allele of the CYP2D6 gene at the small variant position summed to a copy number of the CYP2D6 gene at the small variant position, given (a) a number of sequence reads aligned to the CYP2D6 gene that overlap the small variant position and with bases supporting the small variant allele of the CYP2D6 gene at the small variant position and (b) a number of sequence reads aligned to the CYP2D6 gene that overlap the small variant position and with bases supporting the reference allele of the CYP2D6 gene at the small variant position; and determining one or more small variants the CYP2D6 gene using the possible copy number of the small variant allele of the CYP2D6 gene of the most likely combination determined. In some embodiments, the method comprises: for each of a plurality of small variant positions of the CYP2D6 gene, the small variant position being associated with a small variant allele of the CYP2D6 gene, determining a most likely combination of a possible copy number of the small variant allele of the CYP2D6 gene at the small variant position and a possible copy number of a reference allele of the CYP2D6 gene at the small variant position summed to a copy number of the CYP2D6 gene at the small variant position, given (a) a number of sequence reads aligned to the CYP2D6 gene that overlap the small variant position and with bases supporting the small variant allele of the CYP2D6 gene at the small variant position and (b) a number of sequence reads aligned to the CYP2D6 gene that overlap the small variant position and with bases supporting the reference allele of the CYP2D6 gene at the small variant position; and determining one or more small variants the CYP2D6 gene using the possible copy numbers of the small variant alleles of the CYP2D6 gene of the most likely combinations at the plurality of small variant positions determined.

In some embodiments, the small variant position is in a CYP2D6/CYP2D7 homology region, determining the most likely combination comprises determining the most likely combination of the possible copy number of the small variant allele of the CYP2D6 gene at the small variant position and the possible copy number of the reference allele of the CYP2D6 gene at the small variant position summed to the copy number of the CYP2D6 gene at the small variant position given (a) a number of sequence reads aligned to the CYP2D6 gene or CYP2D7 gene with bases supporting the small variant allele of the CYP2D6 gene at the small variant position and/or (b) a number of sequence reads aligned to the CYP2D6 gene or CYP2D7 gene with bases supporting the reference allele of the CYP2D6 at the small variant position. In some embodiments, the small variant position is not in a CYP2D6/CYP2D7 homology region, determining the most likely combination comprises determining the most likely combination of the possible copy number of the small variant allele of the CYP2D6 gene at the small variant position and the possible copy number of the reference allele of the CYP2D6 gene at the small variant position summed to the copy number of the CYP2D6 gene at the small variant position given (a) a number of sequence reads aligned to the CYP2D6 gene and not to the CYP2D7 gene with bases supporting the small variant allele of the CYP2D6 gene at the small variant position and/or (b) a number of sequence reads aligned to the CYP2D6 gene and not CYP2D7 gene with bases supporting the reference allele of the CYP2D6 at the small variant position.

In some embodiments, the method comprises determining the copy number of the CYP2D6 gene at the small variant position. The copy number of the CYP2D6 gene at the small variant position can comprise a copy number of the CYP2D6 gene. The copy number of the CYP2D6 gene at the small variant position can comprise a copy number of the CYP2D6 gene of possible copy numbers of the CYP2D6 gene of the most likely combinations determined. The copy number of the CYP2D6 gene at the small variant position can comprise a copy number of the CYP2D6 gene of possible copy numbers of the CYP2D6 gene of the most likely combinations determined and closest to the small variant position. The copy number of the CYP2D6 gene at the small variant position can comprise a copy number of the CYP2D6 gene at a 5′ position or 3′ position of the small variant position. In some embodiments, the method comprises: (a) determining the number of sequence reads with bases supporting the small variant allele of the CYP2D6 gene; and (b) determining the number of sequence reads with bases supporting the reference allele of the CYP2D6 gene.

In some embodiments, determining the allele of the CYP2D6 gene the subject has comprises: determining alleles (e.g., 2, 3, 4, 5, or more alleles) of the CYP2D6 gene the subject has. In some embodiments, determining the allele of the CYP2D6 gene the subject has comprises: determining a star allele and/or a haplotype of the CYP2D6 gene the subject has using the one or more structural variants of the CYP2D6 gene determined and/or the one or more small variants of the CYP2D6 gene determined, optionally the star allele is associated with a known function.

In some embodiments, the method comprises: determining a level of CYP2D6 enzymatic activity in the subject using the allele of the CYP2D6 gene determined. The enzymatic activity can be poor, intermediate, normal, or ultrarapid. In some embodiments, the method comprises determining a dosage recommendation of a treatment and/or a treatment recommendation for the subject based on the allele of the CYP2D6 gene the subject has.

Disclosed herein include systems for paralog genotyping. In some embodiments, a system for paralog genotyping comprises: non-transitory memory configured to store executable instructions and sequence data comprising a plurality of sequence reads obtained from a sample of a subject aligned to a first paralog or a second paralog. The system can comprise: a processor (such as a hardware processor or a virtual processor) in communication with the non-transitory memory, the processor programmed by the executable instructions to perform: determining a copy number of paralogs of a first type using a Gaussian mixture model comprising a plurality of Gaussians each representing a different integer copy number given (i) a first number of sequence reads aligned to a first region. The hardware processor programmed by the executable instructions to perform: for one of a plurality of first paralog-specific bases, determining a most likely combination, of a plurality of possible combinations each comprising a possible copy number of a first paralog of the first type and a possible copy number of a second paralog of the first type summed to the copy number of the paralogs of the first type determined, given (a) a number of sequence reads of the plurality of sequence reads with bases that support the first paralog-specific base and (b) a number of sequence reads of the plurality of sequence reads with bases that support a second paralog-specific base of the second paralog corresponding to the first paralog-specific base. The hardware processor programmed by the executable instructions to perform: determining a copy number or an allele of the first paralog using the most likely combination of the possible copy number of the first paralog and the possible copy number of the second paralog determined for the first paralog-specific base. In some embodiments, the first paralog and the second paralog have a sequence identity of at least 90%.

In some embodiments, the hardware processor is programmed by the executable instructions to perform: determining (i) a first number of sequence reads of a plurality of sequence reads in sequence data obtained from a sample of a subject aligned to the first region. The method can comprise: determining (i) a first normalized number of the sequence reads aligned to the first region using (i) a length of the first region, wherein determining the copy number of the first type of paralogs comprises: determining the copy number of the first type of paralogs using the Gaussian mixture model given (i) the first normalized number of the sequence reads aligned to the first region. The hardware processor can be programmed by the executable instructions to perform: can comprise: receiving the sequence data comprising the plurality of sequence reads aligned to the first region.

In some embodiments, the hardware processor is programmed by the executable instructions to perform: determining a copy number of one or more paralogs of a second type using the Gaussian mixture given (ii) a second number of sequence reads aligned to a second region. Determining the copy number or the allele of the first paralog can comprise: determining the copy number or the allele of the first paralog using the most likely combination of the possible copy number of the first paralog and the possible copy number of the second paralog determined for the first paralog-specific base and the copy number of the one or more paralogs of the second type. The method can comprise: determining a copy number of paralogs of a third type from the copy number of the paralogs of the first type and the copy number of the paralogs of the second type. Determining the copy number or the allele of the first paralog comprises: determining the copy number or the allele of the first paralog using the most likely combination of the possible copy number of the first paralog and the possible copy number of the second paralog determined for the first paralog-specific base.

In some embodiments, the first paralog is survival of motor neuron 1 (SMN1) gene. The second paralog can be survival of motor neuron 2 (SMN2) gene. The first region can comprise at least one exon 1 to exon 6 of the SMN1 gene and at least one exon 1 to exon 6 of the SMN2 gene. The second region can comprise at least one of exon 7 and exon 8 of the SMN1 gene and at least one of exon 7 and exon 8 of the SMN2 gene. The paralogs of the first type can comprise an intact SMN1 gene and an intact SMN2 gene. The one or more paralogs of the second type can comprise the intact SMN1 gene, the intact SMN2 gene, a truncated SMN1 gene, or a truncated SMN2 gene. The copy number of the first paralog can comprise a copy number of the SMN1 gene.

In some embodiments, the first paralog is Cytochrome P450 Family 2 Subfamily D Member 6 (CYP2D6) gene. The second paralog can be Cytochrome P450 Family 2 Subfamily D Member 7 (CYP2D7) gene. The first region can comprise the CYP2D6 gene and the CYP2D7 gene. The second region can comprise a spacer region between the CYP2D7 gene and a repetitive element REP7 downstream of the CYP2D7 gene. The paralogs of the first type can comprise the CYP2D6 gene and the CYP2D7 gene. The one or more paralogs of the second type can comprise a CYP2D6/CYP2D7 fusion allele with the spacer region and the repetitive element REP7 downstream of the CYP2D6/CYP2D7 fusion allele. The copy number of first paralog can comprise an allele of the CYP2D6 gene the subject has is a small variant or a structural variant of the CYP2D6 gene.

Disclosed herein include embodiments of a system (e.g., a computing system) comprising non-transitory memory configured to store executable instructions; and a processor (e.g., a hardware processor or a virtual processor) in communication with the non-transitory memory, the hardware processor programmed by the executable instructions to perform any method disclosed herein. Disclosed herein include embodiments of a device (e.g., an electronic device) comprising non-transitory memory configured to store executable instructions; and a processor (e.g., a hardware processor or a virtual processor) in communication with the non-transitory memory, the hardware processor programmed by the executable instructions to perform any method disclosed herein. Disclosed herein include embodiments of a computer readable medium comprising executable instructions that, when executed by a processor (e.g., a hardware processor or a virtual processor) of a system or a device, cause the hardware processor to perform any method disclosed herein.

Details of one or more implementations of the subject matter described in this specification are set forth in the accompanying drawings and the description below. Other features, aspects, and advantages will become apparent from the description, the drawings, and the claims. Neither this summary nor the following detailed description purports to define or limit the scope of the inventive subject matter.

### BRIEF DESCRIPTION OF THE DRAWINGS

FIGS. 1A-1E show causes of SMA and SMN copy number calling according to one embodiment of a method disclosed herein.

FIGS. 2A-2C show population distributions of SMN1/2 copy numbers determined using one embodiment of a method disclosed herein.

FIG. 3 shows SMA identified in two trios in the Next Generation Children project and validated using MLPA.

FIG. 4 shows population frequencies determined using one embodiment of a method disclosed herein agree with previous studies.

FIG. 5 is a non-limiting exemplary IGV snapshot showing CYP2D6 is highly polymorphic and is downstream of CYP2D7, a pseudogene paralog of CYP2D6.

FIG. 6 is a non-limiting exemplary schematic illustration of CYP2D6/7 gene deletions, duplications and fusion genes.

FIG. 7 is a non-limiting exemplary plot showing that allele frequencies determined by the method agree with PharmVar Database from the Pharmacogene Variation (PharmVar) Consortium.

FIG. 8 is a flow diagram showing an exemplary method of determining a copy number of survival of motor neuron 1 (SMN1) gene using sequencing data.

FIG. 9 is a flow diagram showing an exemplary method of genotyping cytochrome P450 family 2 subfamily D member 6 (CYP2D6) gene using sequencing data.

FIG. 10 is a flow diagram showing an exemplary method of paralog genotyping using sequencing data.

FIG. 11 is a block diagram of an illustrative computing system configured to implement paralog genotyping using sequencing data.

FIGS. 12A and 12B show non-limiting exemplary plots illustrating common CNVs affecting the SMN1/SMN2 loci. FIG. 12A shows depth profiles across the SMN1/SMN2 regions. Samples with a total SMN1+SMN2 copy number of 2, 3, 4 and 5 are shown dots, respectively. Depth from 50 samples are summed up for each CN category. Each dot represents normalized depth values in a 100 bp window. Read counts are calculated in each 100 bp window, summing up reads from both SMN1 and SMN2, and normalized to the depth of wild-type samples (CN=4). The SMN exons are represented as purple boxes. The two x axes show coordinates in SMN1 (bottom) and SMN2 (upper). FIG. 12B shows depth profiles aggregated from 50 samples carrying a deletion of exons 7 and 8 are shown as dots. Read depths are calculated in the same way as in FIG. 12A.

FIG. 13 shows a non-limiting exemplary scatterplot of total SMN (SMN1+SMN2) copy number (x axis, called by read depth in Exon 1-6) and intact SMN copy number (y axis, called by read depth in Exon 7-8).

FIGS. 14A-14D illustrate the distributions of SMN1/SMN2/SMN* copy numbers in the population. FIG. 14A is a non-limiting exemplary plot illustrating the percentage of samples showing CN call agreement with c.840C>T across 16 SMN1-SMN2 base difference sites in African and non-African populations. Site 13* is the c.840C>T splice variant site. The black horizontal line denotes 85% concordance. FIG. 14B shows non-limiting exemplary histograms of the distributions of SMN1, SMN2 and SMN* copy numbers across five populations in 1kGP and the NIHR BioResource cohort (numbers shown in Table 15). FIG. 14C is a non-limiting exemplary plot of SMN1 CN vs. total SMN2 CN (intact SMN2+SMN*). FIG. 14D shows two trios with an SMA proband detected by the caller and orthogonally confirmed in the NIHR BioResource cohort. CNs per allele of SMN1, SMN2 and SMN* are phased and labeled for each member of the trios.

FIG. 15 shows non-limiting exemplary plots each illustrating a distribution of posterior probability for simulated SMN1 CN using a single site at different read depths and SMN1:SMN2 CN combinations

FIG. 16 shows a non-limiting exemplary IGV snapshot of the SMN2 region in a sample with the exon 7-8 deletion. Horizontal lines join two reads in a pair in the center alignment track. BLAT results of two split reads spanning the breakpoint are shown in the bottom track, showing two segments of the same read aligning to either side of the deletion breakpoint.

FIG. 17 shows non-limiting exemplary plots illustrating correlation between raw SMN1 CNs at 15 base differences near c840.C>T and raw SMN1 CNs at the c840.C>T site. The raw SMN1 CN at each site was calculated as the CN of intact SMN times the fraction of SMN1 supporting read counts out of SMN1+SMN2 supporting read counts. Correlation coefficients are listed in the title of each plot.

FIGS. 18A and 18B show non-limiting exemplary plots illustrating SMN1/SMN2 haplotypes in samples with SMN1:2 SMN2:0 and SMN1:2 SMN2:1 in 1kGP. The y axis shows the raw SMN1 CNs as defined in FIG. 16. The x axis shows the 16 sites whose indices are listed and explained in Table 8. Index #13 represents the c840.C>T site. Samples with SMN1:2 SMN2:0 are shown together in the upper left plot. Samples with SMN1:2 SMN2:1 are shown as 5 clusters. FIG. 18A. Non-Africans. FIG. 18B. Africans.

FIG. 19 shows a non-limiting exemplary IGV snapshot showing a 1.9 kb deletion in SMN1 in MB509.

FIG. 20 shows a non-limiting exemplary plot illustrating SMN1/SMN2/SMN* CNs in 1kGP and NIHR cohorts.

FIGS. 21A and 21B show discrepancies and no-calls in validation samples.

FIG. 22 shows CN calls derived from BWA and Isaac BAMs.

FIG. 23 is a non-limiting exemplary plot showing WGS data quality in CYP2D6/7 region. Mean mapping quality across 1kGP samples are plotted for each position in the CYP2D6/7 region. A median filter is applied in a 200 bp window. REP6, REP7, and the 9 exons of CYP2D6/7 are shown as boxes on the left (CYP2D6) and right (CYP2D7) boxes. Two 2.8 kb repeat regions downstream of CYP2D6 (REP6) and CYP2D7 (REP7) are identical and essentially unalignable. The dotted box denotes the spacer region between CYP2D7 and REP7. Two major homology regions within the genes are shaded.

FIG. 24 shows structural variants validated by PacBio CCS reads. PacBio reads supporting deletion (*5), duplications, and fusions (*36, *68 and *13). Plots were generated using sv-viz2 (zotero.org/google-docs/?xAunA6). For deletions and duplications, due to significant homology in the REP regions, the exact position of the breakpoints within REP is not available. The breakpoints in A and B are for illustration purposes only.

FIG. 25 is a non-limiting exemplary plot showing CYP2D6 allele frequencies across five ethnic populations for ten most common haplotypes with altered CYP2D6 function. One haplotype (*2×2) has increased function, two haplotypes (*4 and *4+*68) have no function, and the remaining haplotypes have decreased function.

FIG. 26 shows that CYP2D6/CYP2D7 base difference sites have high variability in the population. The y axis shows the frequency of samples where the CN of the CYP2D6 base is called at 2 out of all samples that have a total CYP2D6+CYP2D7 CN of 4. The x axis shows genome coordinates in hg38. CYP2D6 exons are drawn as gray boxes above the plot. The black horizontal line denotes the 98% cutoff.

FIG. 27 shows raw CYP2D6 CNs across CYP2D6/7 differentiating sites in examples with SVs. Raw CYP2D6 CN was calculated as the total CYP2D6+CYP2D7 CN multiplied by the ratio of CYP2D6 supporting reads out of CYP2D6 and CYP2D7 supporting reads. The large diamond denotes the copy number of genes that are CYP2D6-derived at the end of the gene (can be complete CYP2D6 or fusion gene ending in CYP2D6), calculated as the total CYP2D6+CYP2D7 CN minus the CN of the CYP2D7 spacer region (see FIG. 23). To detect SVs, a CYP2D6 CN was called at each site and a change in CYP2D6 CN within the gene indicated the presence of SV. For example, in HG01161, the CYP2D6 CN changed from 2 to 1 between Exon 7 and Exon 9, indicating a CYP2D7-CYP2D6 hybrid gene. In HG00553, the CYP2D6 CN changed from 2 to 3 between Exon 1 and Exon 2, indicating a CYP2D6-CYP2D7 hybrid gene.

FIG. 28 shows that PacBio data confirms *10D fusion in HG00421. A sample with *36 (HG00612) is shown in comparison. PacBio reads that contain the fusions are those with shaded bases that represent soft-clips made by the aligner and were derived from CYP2D7 part of the fusion. The fusion breakpoints are close to each other but the breakpoint for *36 is upstream of the base differences in exon 9 (those inside the black box), while the breakpoint for *10D is downstream, leaving the CYP2D6 gene intact.

FIG. 29 shows that PacBio data had a false *61 (CYP2D6/CYP2D7 hybrid) call made by Aldy in HG02622. Expected genotype was *17/*45 but Aldy called *61-like/*78 (both *61 and *78 are star alleles with SVs). PacBio data showed that there was no structural variant in the region (each read aligns completely, with no soft-clips indicating unaligned parts).

FIGS. 30A and 30B show novel *10+*36+*36+*83 haplotype in HG00597. FIG. 30A. Depth plot as in FIG. 27, showing that HG00597 had three copies of *36-like fusions, all of which had a breakpoint in the homology region between Exon 7 and Exon 9. FIG. 30B. IGV screenshot of the PacBio data, showing all the fusion-containing reads, i.e. those that aligned with a soft-clip. One copy of the fusion gene did not have g.42130692G>A, the SNP that was in *36 but not in *83, as shown in the region flanked by two black vertical lines. This copy was *83, and unlike what was reported in PharmVar, this was a fusion gene with REP7 not REP6, otherwise the copy number of the region downstream of exon 9 would be 3 instead of 2 in FIG. 30A.

FIGS. 31A and 31B compare between 1kGP and pharmGKB frequencies. Each dot represents a haplotype with a frequency greater than or equal to 0.5% in either 1kGP or pharmGKB. SV-related haplotypes are marked, including the two haplotypes with the largest deviation (*10+*36 in East-Asians and *4+*68 in Europeans). Other haplotypes with deviated values are annotated (*2, *41, *34, *39, *2, and *29). A diagonal line is drawn for each panel. Correlation coefficients are listed for each population (*10+*36 is excluded in East-Asians and *4+*68 is excluded in Europeans for calculation). FIG. 31B shows values in the low value range (<5%).

FIG. 32 is a non-limiting exemplary IGV snapshot showing de novo assembly of PacBio reads in HG00733 did not include the *68 fusion.

Throughout the drawings, reference numbers may be re-used to indicate correspondence between referenced elements. The drawings are provided to illustrate example embodiments described herein and are not intended to limit the scope of the disclosure.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments can be utilized, and other changes can be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include methods for determining a copy number of survival of motor neuron 1 (SMN1) gene and/or the survival of motor neuron 2 (SMN2) gene. In some embodiments, a method for determining a copy number of the SMN1 gene and/or the SMN2 gene is under control of a processor (such as a hardware processor or a virtual processor) and comprises: receiving sequence data comprising a plurality of sequence reads obtained from a sample of a subject aligned to the SMN1 gene or SMN2 gene. The method can comprise: determining (i) a first number of sequence reads of the plurality of sequence reads aligned to a first SMN1 or SMN2 region comprising at least one of exon 1 to exon 6 of the SMN1 gene or the SMN2 gene, respectively, and (ii) a second number of sequence reads of the plurality of sequence reads aligned to a second SMN1 or SMN2 region comprising at least one of exon 7 and exon 8 of the SMN1 gene or the SMN2 gene, respectively. The method can comprise: determining (i) a first normalized number of the sequence reads aligned to the first SMN1 or SMN2 region and (ii) a second normalized number of the sequence reads aligned to the second SMN1 or SMN2 region using (i) a length of the first SMN1 or SMN2 region and (ii) a length of the second SMN1 or SMN2 region, respectively. The method can comprise: determining (i) a copy number of total survival of motor neuron (SMN) genes, each being an intact SMN1 gene, an intact SMN2 gene, a truncated SMN1 gene, or a truncated SMN2 gene, and (ii) a copy number of any intact SMN genes, each being the intact SMN1 gene or the intact SMN2 gene, using a Gaussian mixture model comprising a plurality of Gaussians each representing a different integer copy number, given (i) the first normalized number of the sequence reads aligned to the first SMN1 or SMN2 region and (ii) the second normalized number of the sequence reads aligned to the second SMN1 or SMN2 region, respectively. The method can comprise: for one of a plurality of SMN1 gene-specific bases associated with the intact SMN1 gene, determining a most likely combination, of a plurality of possible combinations each comprising a possible copy number of the SMN1 gene and a possible copy number of the SMN2 gene summed to the copy number of any intact SMN genes determined, given (a) a number of sequence reads of the plurality of sequence reads with bases that support the SMN1 gene-specific base and (b) a number of sequence reads of the plurality of sequence reads with bases that support a SMN2 gene-specific base of the SMN2 gene corresponding to the SMN1 gene-specific base. The method can comprise: determining a copy number of the SMN1 gene and/or the SMN2 gene using the most likely combination of the possible copy number of the SMN1 gene and the possible copy number of the SMN2 gene determined for the SMN1 gene-specific base.

Disclosed herein includes methods for genotyping cytochrome P450 family 2 subfamily D member 6 (CYP2D6) gene. In some embodiments, a method for genotyping the CYP2D6 gene is under control of a processor (such as a hardware processor or a virtual processor) and comprises: receiving sequence data comprising a plurality of sequence reads obtained from a sample of a subject aligned to the CYP2D6 gene or cytochrome P450 Family 2 Subfamily D Member 7 (CYP2D7) gene. The method can comprise: determining (i) a first number of sequence reads of the plurality of sequence reads aligned to the CYP2D6 gene or the CYP2D7 gene. The method can comprise: determining (i) a first normalized number of the sequence reads aligned to the CYP2D6 gene or the CYP2D7 gene using (i) a length of the CYP2D6 gene or the CYP2D7 gene, respectively. The method can comprise: determining (i) a total copy number of the CYP2D6 gene and the CYP2D7 gene using a Gaussian mixture model comprising a plurality of Gaussians each representing a different integer copy number, given (i) the first normalized number of the sequence reads aligned to the CYP2D6 gene or the CYP2D7 gene. The method can comprise: for one of a plurality of CYP2D6 gene-specific bases, determining a most likely combination, of a plurality of possible combinations each comprising a possible copy number of the CYP2D6 gene and a possible copy number of the CYP2D7 gene summed to the total copy number of the CYP2D6 gene and the CYP2D7 gene determined, given (a) a number of sequence reads of the plurality of sequence reads with bases that support the CYP2D6 gene-specific base and (b) a number of sequence reads of the plurality of sequence reads with bases that support a CYP2D7 gene-specific base of corresponding to the CYP2D6 gene-specific base. The method can comprise: determining an allele of the CYP2D6 gene the subject has using the most likely combination of the possible copy number of the CYP2D6 gene and the possible copy number of the CYP2D7 gene determined for the CYP2D6 gene-specific base.

Disclosed herein include methods for paralog genotyping. In some embodiments, a method for paralog genotyping is under control of a processor (such as a hardware processor or a virtual processor) and comprises: receiving sequence data comprising a plurality of sequence reads obtained from a sample of a subject aligned to a first paralog or a second paralog. The method can comprise: determining a copy number of paralogs of a first type using a Gaussian mixture model comprising a plurality of Gaussians each representing a different integer copy number given (i) a first number of sequence reads aligned to a first region. The method can comprise: for one of a plurality of first paralog-specific bases, determining a most likely combination, of a plurality of possible combinations each comprising a possible copy number of a first paralog of the first type and a possible copy number of a second paralog of the first type summed to the copy number of the paralogs of the first type determined, given (a) a number of sequence reads of the plurality of sequence reads with bases that support the first paralog-specific base and (b) a number of sequence reads of the plurality of sequence reads with bases that support a second paralog-specific base of the second paralog corresponding to the first paralog-specific base. The method can comprise: determining a copy number or an allele of the first paralog using the most likely combination of the possible copy number of the first paralog and the possible copy number of the second paralog determined for the first paralog-specific base.

Disclosed herein include embodiments of a system (e.g., a computing system) comprising non-transitory memory configured to store executable instructions; and a processor (e.g., a hardware processor or a virtual processor) in communication with the non-transitory memory, the hardware processor programmed by the executable instructions to perform any method disclosed herein. Disclosed herein include embodiments of a device (e.g., an electronic device) comprising non-transitory memory configured to store executable instructions; and a processor (e.g., a hardware processor or a virtual processor) in communication with the non-transitory memory, the hardware processor programmed by the executable instructions to perform any method disclosed herein. Disclosed herein include embodiments of a computer readable medium comprising executable instructions that, when executed by a processor (e.g., a hardware processor or a virtual processor) of a system or a device, cause the hardware processor to perform any method disclosed herein.

Spinal Muscular Atrophy Diagnosis and Carrier Screening from Whole Genome Sequencing Data

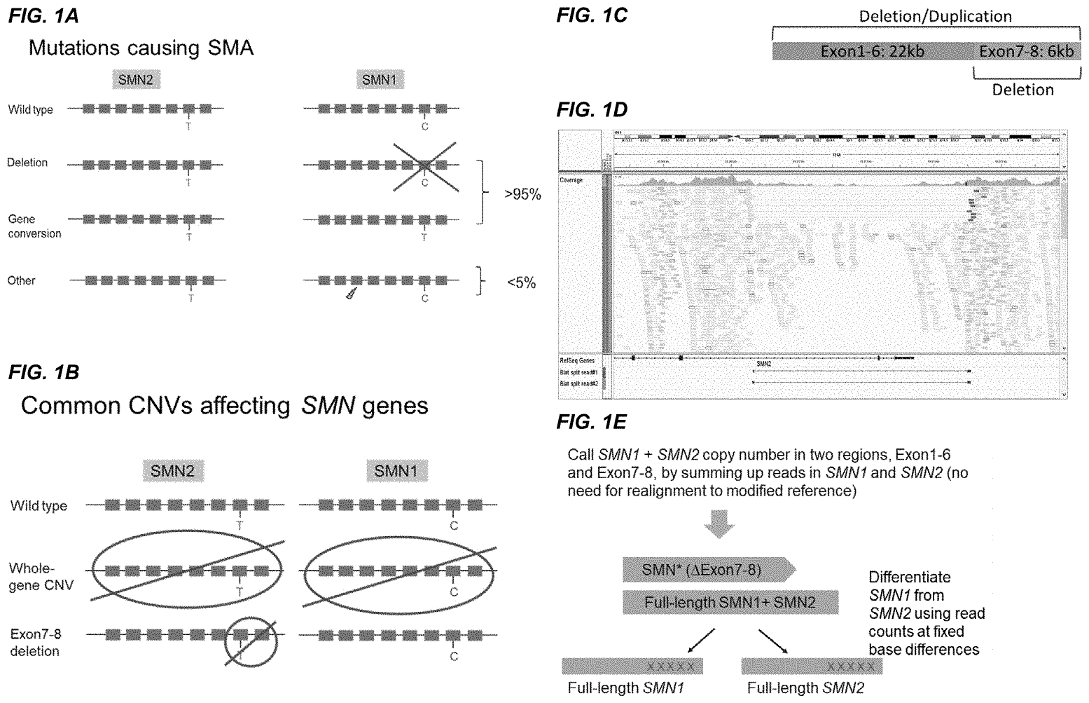

Spinal muscular atrophy (SMA) is characterized by a weakness of the voluntary muscles and is a leading genetic cause of early childhood death with an incidence of 1 in 6000-10,000 live births and a carrier frequency of 1:40-801,2. SMA is caused by mutations in the SMN1 (survival motor neuron 1) gene (FIG. 1A). A duplicated gene SMN2 differs with SMN1 by just a few base pairs, one of which, the c.840C>T splice variant in exon 7, has a functional consequence. By interrupting a splicing enhancer, the c.840C>T mutation leads to increased skipping of exon 7 and reduction of full-length transcripts in SMN23 (FIGS. 1B-1D). The genomic region is subject to unequal crossing-over and gene conversion, resulting in variable copy numbers of SMN1 and SMN2 (FIG. 1B). Due to the high incidence rate and disease severity, population-wide SMA screening is recommended and the key to this screening is to determine the copy number of SMN1, for SMA diagnosis and carrier testing. Additionally, the copy number of SMN2 defines the severity of SMA and is important for clinical classification and prognosis.

Conventional SMA carrier tests use PCR-based methods, such as multiplex ligation-dependent probe amplification (MLPA), quantitative PCR (qPCR) and digital PCR. These methods mainly target the c.840C>T site. Incorporating SMA screening into high-throughput NGS-based tests that can simultaneously profile a large number of genes or even the entire genome can be advantageous. The almost perfect sequence identity between SMN1 and SMN2 makes variant calling challenging with standard NGS-based methods.

Disclosed herein is a SMN copy number caller based on a bioinformatics method that determines the copy number of SMN1 and SMN2 with whole genome sequencing (WGS) data (FIG. 1E). The method can include calling SMN1+SMN2 copy number in two regions, Exon1-6 and Exon7-8, by summing up reads in SMN1 and SMN2. The method can include differentiating SMN1 from SMN2 using read counts at fixed base differences. In some embodiments, the method does not include realignment of aligned sequences to a modified reference. The method is the first SMN copy number calling tool that can identify both patients with SMA and carriers from WGS data. Some embodiments of the method are not restricted to exons 7 and 8 and do not focus primarily on c.840C>T. The method takes a gene-wide approach and provides the most comprehensive call set, including the copy number of full-length SMN1 and SMN2, as well as a truncated form of SMN with a deletion of exons 7 and 8. This method can be readily applicable to any WGS data and will be a valuable tool for SMA diagnosis and carrier screening to be incorporated into high-throughput population-wide WGS screening.

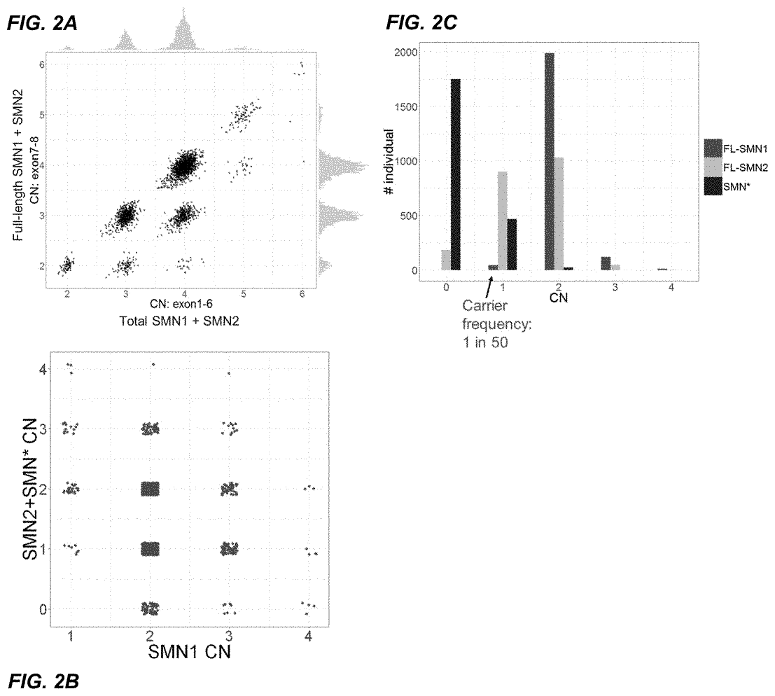

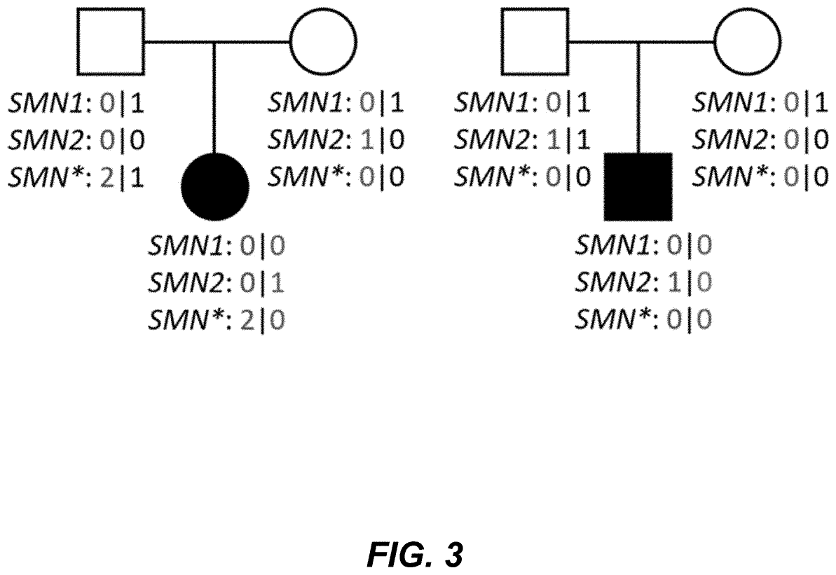

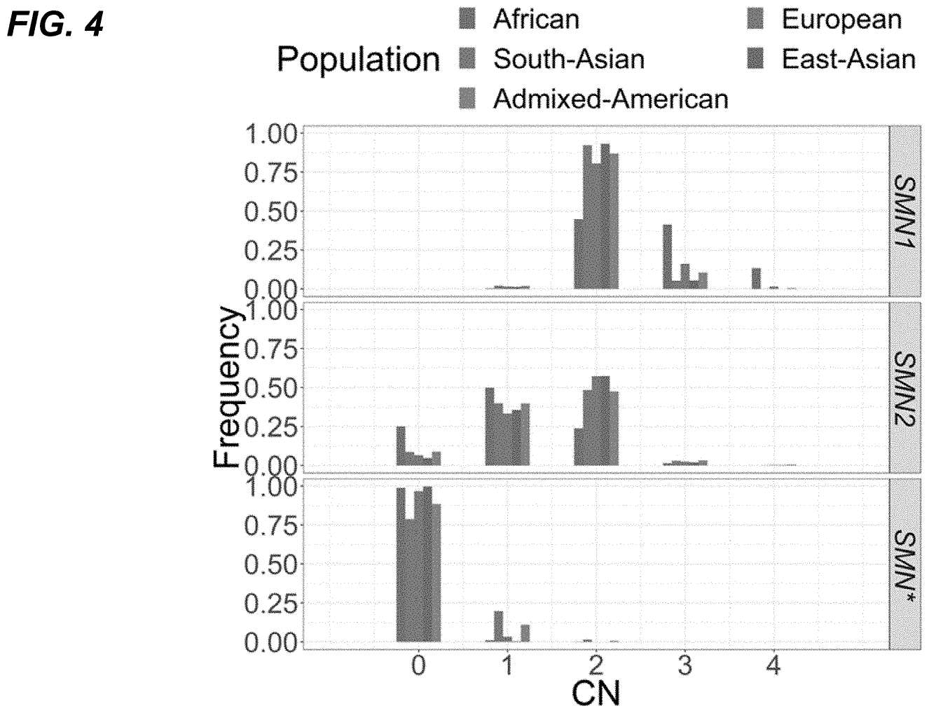

FIGS. 1A-1E show causes of SMA SMN copy number calling according to one embodiment of a bioinformatics method disclosed herein. Table 1 shows differentiating SMN1 from SMN2 based on fixed single nucleotide polymorphism (SNPs) according to the embodiment of the method. SMN1 copy number calls are made in 16 sites near c.840C>T. Nine sites with a high percent agreement with c.840C>T are selected to make a joint call on SMN1 copy number. FIGS. 2A-2C and Table 2 show population distributions of SMN1/2 copy numbers determined. More copies of SMN1 were observed when there were fewer copies of SMN2, suggesting gene conversion as a mechanism for the CN variability of SMN1 and SMN2. Table 3 shows validation of copy numbers calling determined using the bioinformatics method against copy numbers determined using digital PCR. Validation against digital PCR showed 100% agreement in SMN1 CN and 98% agreement in SMN2 CN. FIG. 3 shows SMA identified in two trios in the Next Generation Children project and validated using MLPA. FIG. 4 and Table 4 show population frequencies determined using the bioinformatics method agree with previous studies.

TABLE 1
Differentiating SMN1 from SMN2 based on fixed single nucleotide polymorphism (SNPs).

| | | | SMN1 | | SMN2 | | Percent agreement |
|---|---|---|---|---|---|---|---|
| Site # | Location | Selected | Position, hg19 | Base | Position, hg19 | Base | with c.840C > T |
| 1 | Intron 6 | | 70244142 | A | 69368717 | G | 85.2 |
| 2 | Intron 6 | | 70245876 | T | 69370451 | C | 85.5 |
| 3 | Intron 6 | | 70246016 | G | 69370591 | A | 94.8 |
| 4 | Intron 6 | | 70246019 | T | 69370594 | C | 94.1 |
| 5 | Intron 6 | | 70246156 | G | 69370731 | A | 93.9 |
| 6 | Intron 6 | | 70246167 | T | 69370742 | C | 59.8 |
| 7 | Intron 6 | | 70246320 | G | 69370895 | A | 96.3 |
| 8 | Intron 6 | Yes | 70246793 | G | 69371368 | A | 99 |
| 9 | Intron 6 | Yes | 70246919 | A | 69371499 | C | 98.2 |
| 10 | Intron 6 | Yes | 70247219 | G | 69371799 | A | 98.8 |
| 11 | Intron 6 | Yes | 70247290 | T | 69371870 | C | 99 |
| 12 | Intron 6 | Yes | 70247724 | G | 69372304 | A | 99.6 |
| 13 | Exon 7 | Yes | 70247773 | C | 69372353 | T | 100 |
| | (c.840C > T) | | | | | | |
| 14 | Intron 7 | Yes | 70247921 | A | 69372501 | G | 99.5 |
| 15 | Intron 7 | Yes | 70248036 | A | 69372616 | G | 99.6 |
| 16 | Exon 8 | Yes | 70248501 | G | 69373081 | A | 97.9 |

TABLE 2
Population distributions of SMN1/2 copy numbers

| # individuals | | |
|---|---|---|
| N = 2235 | Full length SMN1 | Full length SMN2 |
| 9 | 1 | 1 |
| 24 | 1 | 2 |
| 10 | 1 | 3 |
| 2 | 1 | 4 |
| 162 | 2 | 0 |
| 827 | 2 | 1 |
| 1005 | 2 | 2 |
| 45 | 2 | 3 |
| 1 | 2 | 4 |
| 15 | 3 | 0 |
| 75 | 3 | 1 |
| 41 | 3 | 2 |
| 7 | 3 | 3 |
| 1 | 3 | 4 |
| 5 | 4 | 0 |
| 3 | 4 | 1 |
| 3 | 4 | 2 |

TABLE 3
Validation of copy numbers calling determined using the bioinformatics
method against copy numbers determined using digital PCR.

| | | SMN copy number caller | | | | | |
|---|---|---|---|---|---|---|---|
| Sample | SMA | Full-length | Full-length | | Digital PCR | | |
| ID | Status | SMN1 CN | SMN2 CN | SMN* CN | SMN1 CN | SMN2 CN | Agree |
| NA03813 | Affected | 0 | 3 | 0 | 0 | 3 | Yes |
| NA09677 | Affected | 0 | 3 | 0 | 0 | 3 | Yes |
| NA23689 | Affected | 0 | 3 | 0 | 0 | 3 | Yes |
| NA00232 | Affected | 0 | 2 | 0 | 0 | 2 | Yes |
| NA10684 | Affected | 0 | 2 | 0 | 0 | 2 | Yes |
| NA23687 | Carrier | 1 | 2 | 0 | 1 | 2 | Yes |
| NA23688 | Carrier | 1 | 2 | 0 | 1 | 2 | Yes |
| NA03815 | Carrier | 1 | 1 | 0 | 1 | 1 | Yes |

TABLE 4
Population frequencies determined using the bioinformatics
method agree with previous studies.

| | | Carrier | | |
|---|---|---|---|---|
| | | SMN1 | SMN1 | SMN1 |
| | frequency (%) | CN = 1 | CN = 2 | CN >= 3 |
| Caucasians | This study | 2.2 | 92.2 | 5.6 |
| | Hendrickson et al.^{a} | 2.7 | 91 | 6.3 |
| | Sugarman et al.^{b} | 2.02 | 90.9 | 7.05 |
| Africans | This study | 0.44 | 44.8 | 54.8 |
| | Hendrickson et al.^{a}* | 1.1 | 52.1 | 46.8 |
| | Sugarman et al.^{b}* | 0.98 | 51.9 | 47.1 |

^{a}Hendrickson et al. Differences in SMN1 allele frequencies among ethnic groups within North America. J Med Genet. 2009; 46(9): 641-644. doi: 10.1136/jmg.2009.066969.
^{b}Sugarman et al. Pan-ethnic carrier screening and prenatal diagnosis for spinal muscular atrophy: clinical laboratory analysis of >72 400 specimens. Eur J Hum Genet. 2012; 20(1): 27-32. doi: 10.1038/ejhg.2011.134.
*African Americans

### Characterization of Medically Actionable Variants in 2,500 Publicly-Available, High-Depth Genomes of Diverse Ancestry

Population-scale whole genome sequencing (WGS) data is increasingly available. For example, public sequence data such as the high depth (>30×) WGS data from >2,500 samples from the 1000 Genomes Project (1kGP) is available. This has greatly improved clinical interpretation of simple single nucleotide variations (SNVs) and insertions/deletions (indels). Yet, many medically-important regions and variants such as triplet repeats and homologs are not included in WGS-based databases because annotating these regions and variants require specialized bioinformatics methods. To this effect, population-level characterization of known clinical variants is needed to maximize the impact of population sequencing experiments. In some embodiments, methods disclosed herein address three shortcomings of the standard secondary analysis pipelines: 1) spinal muscular atrophy (SMA) detection and carrier screening, 2) CYP2D6 genotyping for pharmacogenomics applications and 3) detection of triplet repeat expansions. The methods can be targeted can used to call the SMN1/2 copy number, CYP2D6 star alleles, and repeat expansions in the 1kGP population and quantify differences between subpopulations. The frequency distributions by sub-population and perpendicular validation of these methods using validation data generated from high-quality long reads are described herein.

CYP2D6

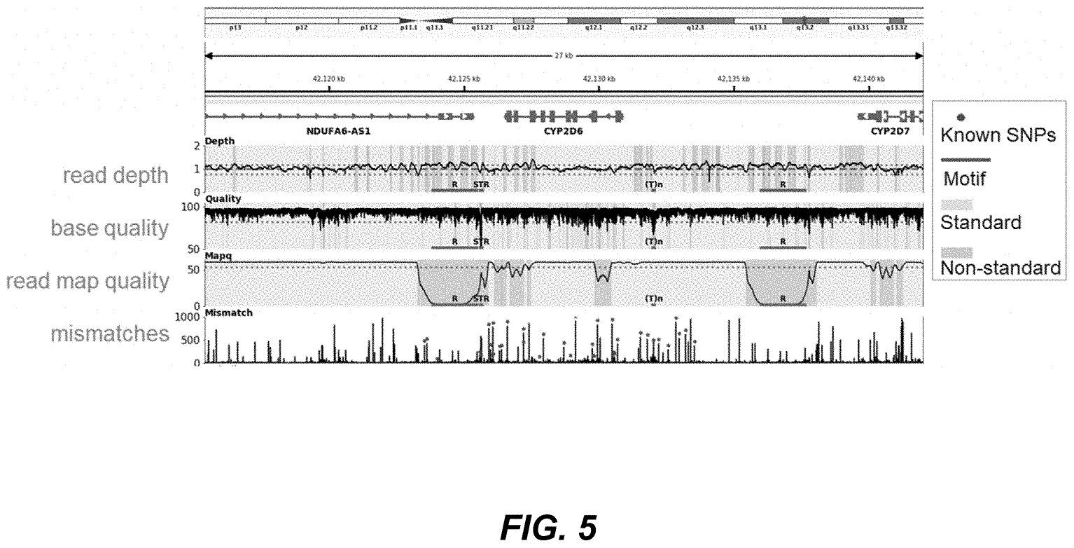

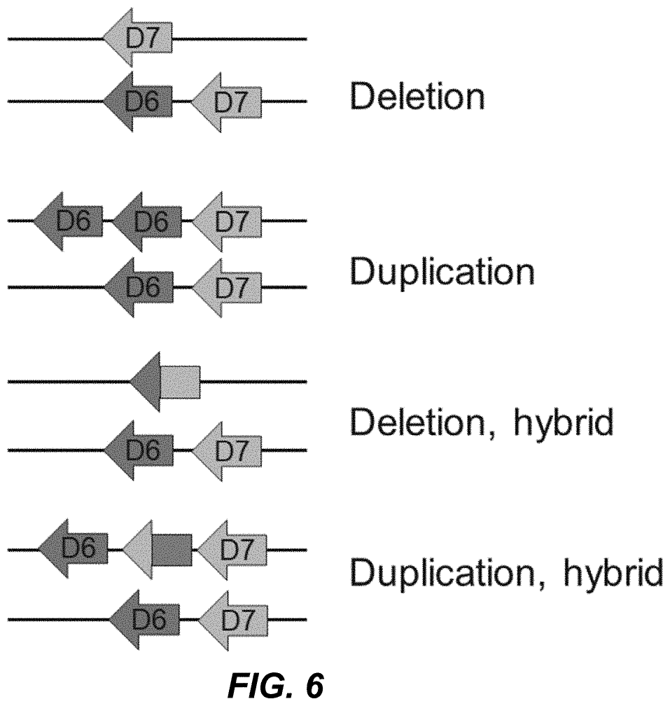

CYP2D6 is an important drug-metabolizing enzyme that is highly polymorphic (FIG. 5). CYP2D6 has high sequence similarity with its pseudogene paralog (CYP2D7). CYP2D6 genotyping with WGS is challenging due to common gene conversions between CYP2D6 and CYP2D7 (referred to herein CYP2D6/7 hereafter), common SVs (gene deletions, duplications and CYP2D6/7 fusion genes; see FIG. 6 for illustrations), as well as the sequence similarity between CYP2D/7, which results in ambiguous read alignments to either genes (FIG. 5). Disclosed herein is a CYP2D6 caller based on a bioinformatics method that can call (e.g., definitely call) diplotypes targeting star alleles (e.g., all star alleles) with known functions. In some embodiments, the method includes the following actions

1. Call total copy number CYP2D6+CYP2D7.

2. Call CNV/hybrids based on copy number calls across CYP2D6/CYP2D7 differentiating sites.

3. Call 56 SNPs/indels from BAM (or another file containing sequence reads).

Use copy number information.

Count reads at both CYP2D6 and CYP2D7 positions in homology regions.

4. Call star alleles and diplotypes based on all called variants.

Table 5 shows validation results of CYP2D6 star allele calls made by the method. The CYP2D6 star allele calls made by the method for 92 out of 96 samples agree with GeT-RM consensus calls from multiple platforms. The method outperformed callers such as Aldy (CYP2D6 star allele calls for 89 out of 96 samples agree with the GeT-RM consensus) and Stargazer (CYP2D6 star allele calls for 83 out of 96 samples agree with the GeT-RM consensus)

TABLE 5
CYP2D6 Caller Validation.

| Sample | CYP2D6 call | GeT-RM consensus | Aldy | Stargazer |
|---|---|---|---|---|
| NA24008 | *1/*4 + *68 | *1/*4 | *1/*4 + *68 | *1/*4 + *68 |
| NA21781 | *2 × 2/*4 + *68 | *2 × 2/*68 + *2 | *2 × 2/*4 + *68 | *2 × 2/*4 + *68 |
| NA23874 | *4/*4 + *68 | *4/*4 | No-call | *4/*4 + *68 |
| NA18565 | *10/*10 + *36 | *10/*36 × 2 | *10/*10 + *36 | *10/*10 + *36 |

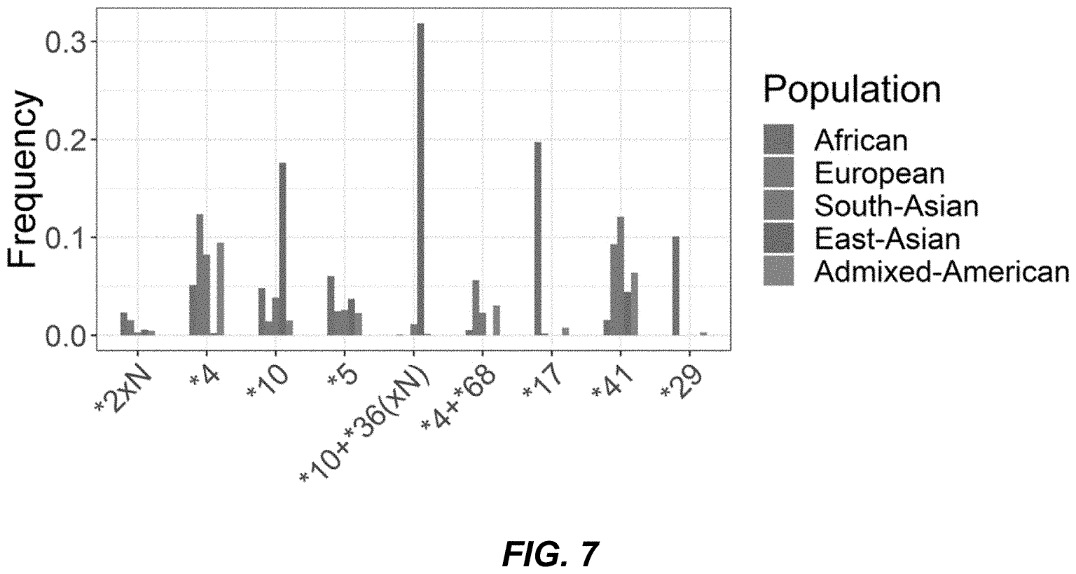

FIG. 7 shows that allele frequencies determined by the method agree with PharmVar Database from the Pharmacogene Variation (PharmVar) Consortium.

### Determining a Copy Number of Survival of Motor Neuron 1 Gene Using Sequencing Data

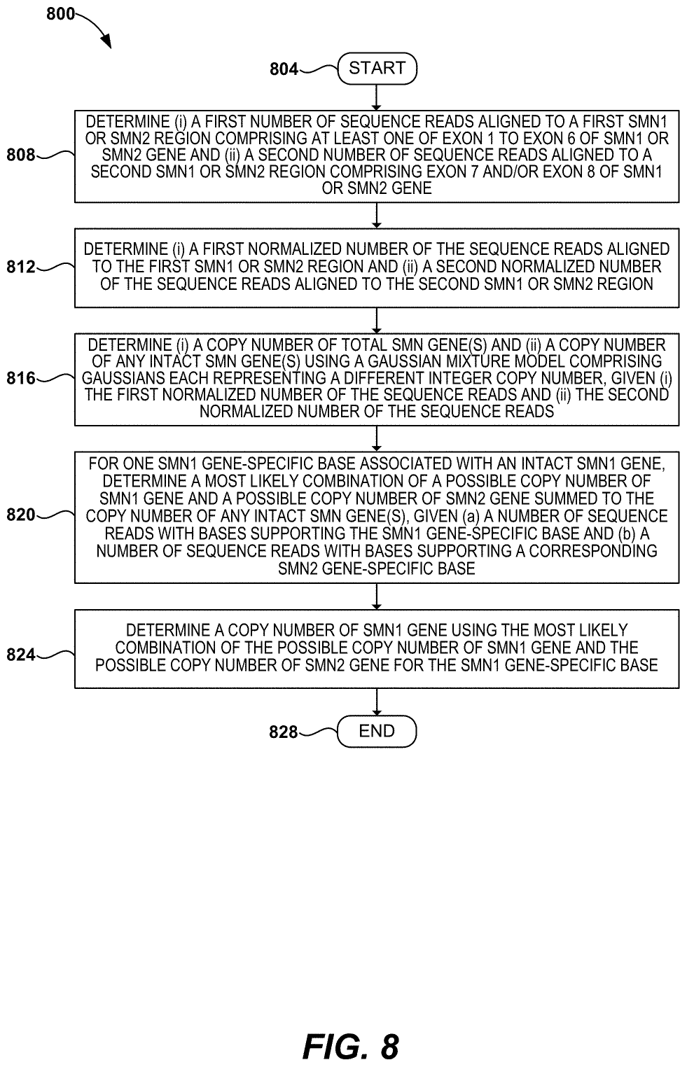

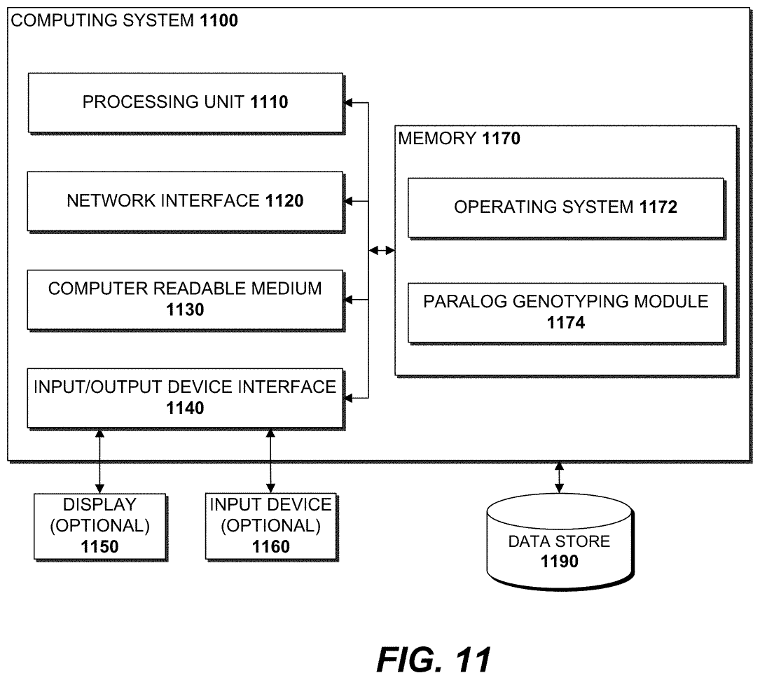

FIG. 8 is a flow diagram showing an exemplary method 800 of determining a copy number of survival of motor neuron 1 gene using sequencing data, such as whole genome sequencing data. The method 800 may be embodied in a set of executable program instructions stored on a computer-readable medium, such as one or more disk drives, of a computing system. For example, the computing system 1100 shown in FIG. 11 and described in greater detail below can execute a set of executable program instructions to implement the method 800. When the method 800 is initiated, the executable program instructions can be loaded into memory, such as RAM, and executed by one or more processors of the computing system 1100. Although the method 800 is described with respect to the computing system 1100 shown in FIG. 11, the description is illustrative only and is not intended to be limiting. In some embodiments, the method 800 or portions thereof may be performed serially or in parallel by multiple computing systems.

After the method 800 begins at block 804, the method 800 proceeds to block 808, where a computing system (such as the computing system 1100 described with reference to FIG. 11) determines (i) a first number of sequence reads of a plurality of sequence reads aligned to a first a survival of motor neuron 1 (SMN1) or a survival of motor neuron 2 (SMN2) region comprising at least one of exon 1 to exon 6 of a SMN1 gene or a SMN2 gene, respectively, and (ii) a second number of sequence reads of the plurality of sequence reads aligned to a second SMN1 or SMN2 region comprising at least one of exon 7 and exon 8 of the SMN1 gene or the SMN2 gene, respectively. The first number of the sequence reads aligned to the first SMN1 or SMN2 region (or the second number of the sequence reads aligned to the second SMN1 or SMN2 region) can be or be about, for example, 5, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, or more.

The at least one of exon 1 to exon 6 of the SMN1 gene can comprise exon 1, exon 2, exon 3, exon 4, exon 5, and/or exon 6 of the SMN1 gene. The at least one of exon 1 to exon 6 of the SMN2 gene can comprise exon 1, exon 2, exon 3, exon 4, exon 5, and/or exon 6 of the SMN2 gene. The first SMN1 or SMN2 region can comprise the exon 1 to the exon 6 of the SMN1 gene or the SMN2 gene, respectively, and can be about 22.2 kb in length. The second SMN1 or SMN2 region can comprise the exon 7 and the exon 8 of the SMN1 gene or the SMN2 gene, respectively, and can be about 6 kb in length.

In some embodiments, the computing system receives sequence data comprising the plurality of sequence reads obtained from a sample of a subject aligned to the SMN1 gene or the SMN2 gene. The sequencing data can comprise whole genome sequencing (WGS) data or short-read WGS data. In some embodiments, the subject is a fetal subject, a neonatal subject, a pediatric subject, an adolescent subject, or an adult subject. The sample can comprise cells or cell-free DNA. The sample can comprise fetal cells or cell-free fetal DNA.

In some embodiments, a sequence read of the plurality of sequence reads is aligned to the first SMN1 or SMN2 region or the second SMN1 or SMN2 region with an alignment quality score of about zero. The alignment quality can be or be about, for example, 0, 0.01, 0.02, 0.03, 0.04, 0.05, 0.06, 0.07, 0.08, 0.09, 0.10, or more (on a scale from 0 to 1 from the alignment score).

The method 800 proceeds from block 808 to block 812, where the computing system determines (i) a first normalized number of the sequence reads aligned to the first SMN1 or SMN2 region and (ii) a second normalized number of the sequence reads aligned to the second SMN1 or SMN2 region using (i) a length of the first SMN1 or SMN2 region and (ii) a length of the second SMN1 or SMN2 region, respectively. The first normalized number of the sequence reads aligned to the first SMN1 or SMN2 region (or the second normalized number of the sequence reads aligned to the second SMN1 or SMN2 region) can be or be about, for example, 1, 2, 3, 4, 5, 6, 7, 9, 10, 20, 30, 40, 50, 60, 70, 80, 90, 100, or more. The length of the first SMN1 or SMN2 region can be or be about, for example, 3 kb, 6 kb, 9 kb, 12 kb, 15 kb, 18 kb, 21 kb, 22.2 kb, 24 kb, or more in length. The length of the second SMN1 or SMN2 region can be or be about, for example, 3 kb, 6 kb, or more in length.

In some embodiments, to determine (i) the first normalized number of the sequence reads aligned to the first SMN1 or SMN2 region and (ii) the second normalized number of the sequence reads aligned to the second region, the computing system can determine (i) the first normalized number of the sequence reads aligned to the first SMN1 or SMN2 region and (ii) the second normalized number of the sequence reads aligned to the second SMN1 or SMN2 region using (i) the length of the first SMN1 or SMN2 region and (ii) the length of the second SMN1 or SMN2 region, respectively, and (iii) a depth of sequence reads of a region of a genome of the subject other than genetic loci comprising the SMN1 gene and the SMN2 gene in the sequence data. The depth of the sequence reads of the region of the genome of the subject other than the genetic loci comprising the SMN1 gene and the SMN2 gene in the sequence data can be or be about, for example, 3, 4, 5, 10, 20, 30, 40, 50, 60, 70, 80, 90, 100 or more.

To determine (i) the first normalized number of the sequence reads aligned to the first SMN1 or SMN2 region and (ii) the second normalized number of the sequence reads aligned to the second SMN1 or SMN2 region, the computing system determines (i) a first SMN1 or SMN2 region-length normalized number of the sequence reads aligned to the first SMN1 or SMN2 region and (ii) a second SMN1 or SMN2 region-length normalized number of the sequence reads aligned to the second SMN1 or SMN2 region using (i) the length of the first SMN1 or SMN2 region and (ii) the length of the second SMN1 or SMN2 region, respectively. The computing system can determine (i) a first normalized depth of the sequence reads aligned to the first region SMN1 or SMN2 and (ii) a second normalized depth of the sequence reads aligned to the second SMN1 or SMN2 region from (i) the first SMN1 or SMN2 region-length normalized number and (ii) the second SMN1 or SMN2 region-length normalized number, respectively, using the depth of the sequence reads of the region of the genome of the subject other than genetic loci comprising the SMN1 gene and the SMN2 gene. The first normalized number of the sequence reads aligned to the first SMN1 or SMN2 region and the second normalized number of the sequence reads aligned to the second SMN1 or SMN2 region can be the first normalized depth and the second normalized depth, respectively.

In some embodiments, to determine (i) the first normalized number of the sequence reads aligned to the first SMN1 or SMN2 region and (ii) the second normalized number of the sequence reads aligned to the second region, the computing system can determine (i) the first normalized number of the sequence reads aligned to the first SMN1 or SMN2 region and (ii) the second normalized number of the sequence reads aligned to the second SMN1 or SMN2 region using (i) a GC content of the first SMN1 or SMN2 region and (ii) a GC content of the second SMN1 or SMN2 region, respectively, and (iii) a depth of sequence reads of a region of a genome of the subject other than genetic loci comprising the SMN1 gene and the SMN2 gene in the sequence data, and (iv) a GC content of the region of the genome. The GC content of the first SMN1 or SMN2 region (or the GC content of the second SMN1 or SMN2 region) can be or be about, for example, 40%, 41%, 42%, 43%, 44%, 45%, 46%, 47%, 48%, 49%, 50%, 51%, 52%, 53%, 54%, 55%, 56%, 57%, 58%, 59%, or 60%. The depth of the sequence reads of the region of the genome of the subject other than the genetic loci comprising the SMN1 gene and the SMN2 gene in the sequence data can be or be about, for example, 3, 4, 5, 10, 20, 30, 40, 50, 100 or more. The GC content of the region of the genome of the subject other than the genetic loci comprising the SMN1 gene and the SMN2 gene in the sequence data can be or be about, for example, 40%, 41%, 42%, 43%, 44%, 45%, 46%, 47%, 48%, 49%, 50%, 51%, 52%, 53%, 54%, 55%, 56%, 57%, 58%, 59%, or 60%.

In some embodiments, the depth of the region comprises an average depth of the sequence reads of the region of the genome of the subject other than the genetic loci comprising the SMN1 gene and the SMN2 gene in the sequencing data. The depth of the region can comprise a median depth of the sequence reads of the region of the genome of the subject other than the genetic loci comprising the SMN1 gene and the SMN2 gene in the sequencing data. The depth of the region can be or b about, for example, 3, 4, 5, 6, 7, 8, 9, 10, 20, 30, 40, 50, 60, 70, 80, 90, 100, or more. The region can comprise about 500, 1000, 1500, 2000, 2500, 3000, 3500, 4000, or more pre-selected regions of about 0.5 kb, 1 kb, 1.5 kb, 2 kb, 2.5 kb, or 3 kb, in length each across the genome of the subject. For example, the region can comprise about 3000 pre-selected regions of about 2 kb in length each across the genome of the subject.

In some embodiments, the first normalized number of the sequence reads aligned to the first SMN1 or SMN2 region (or the second normalized number of the sequence reads aligned to the second SMN1 or SMN2 region) is or is about 10, 20, 30, 40, 50, 60, 70, 80, 90, 100, or more. For example, (i) the first normalized number of the sequence reads aligned to the first SMN1 or SMN2 region and/or (ii) the second normalized number of the sequence reads aligned to the second SMN1 or SMN2 region is about 30 to about 40.

The method 800 proceeds from block 812 to block 816, where the computing system determines (i) a copy number of total survival of motor neuron (SMN) genes and (ii) a copy number of any intact SMN gene(s) using a Gaussian mixture model comprising a plurality of Gaussians each representing a different integer copy number, given (i) the first normalized number of the sequence reads aligned to the first SMN1 or SMN2 region and (ii) the second normalized number of the sequence reads aligned to the second SMN1 or SMN2 region, respectively. The total survival of motor neuron genes can comprise an intact SMN1 gene, an intact SMN2 gene, a truncated SMN1 gene, and/or a truncated SMN2 gene. Any intact SMN gene(s) can comprise the intact SMN1 gene and/or the intact SMN2 gene. The copy number of the total SMN gene(s) (or any gene(s) of the present disclosure) can be or be about, for example, 1, 2, 3, 4, 5, 6, 7, 8, 9, 10, or more. The copy number of any intact SMN gene(s) (or any gene(s) of the present disclosure) can be or be about, for example, 1, 2, 3, 4, 5, 6, 7, 8, 9, 10, or more.

In some embodiments, the Gaussian mixture model comprises a one-dimensional Gaussian mixture model. The plurality of Gaussians of the Gaussian mixture model can represent integer copy numbers, for example, 0 to 5, 0 to 6, 0 to 7, 0 to 8, 0 to 9, 0 to 10, 0 to 11, 0 to 12, 0 to 13, 0 to 14, or 0 to 15. For example, the plurality of Gaussians of the Gaussian mixture model can represent integer copy numbers 0 to 10. A mean (e.g., 1, 2, 3, 4, 5, 6, 7, 8, 9, 10, or more) of each of the plurality of Gaussians can be the integer copy number represented by the Gaussian (e.g., copy numbers of 1, 2, 3, 4, 5, 6, 7, 8, 9, 10, or more). The standard deviation of a Gaussian can be or be about, for example, 0.1, 0.2, 0.3, 0.4, 0.5, 0.6, 0.7, 0.8, 0.9, 1, or more.

In some embodiments, to determine (i) the copy number of the total SMN gene(s) and (ii) the copy number of any intact SMN gene(s), the computing system can determine (i) the copy number of the total SMN gene(s) and (ii) the copy number of any intact SMN gene(s) using the Gaussian mixture model, and a first predetermined posterior probability threshold, given (i) the first normalized number of the sequence reads aligned to the first SMN1 or SMN2 region and (ii) the second normalized number of the sequence reads aligned to the second SMN1 or SMN2 region, respectively. The first predetermined posterior probability threshold (or any predetermined posterior probability threshold of the present disclosure) can be or be about, for example, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, or more. For example, the first predetermined posterior probability threshold can be 0.95.

The method 800 proceeds from block 816 to block 820, where the computing system determines, for one of a plurality of SMN1 gene-specific bases (also referenced to herein as SMN differentiating bases) associated with the intact SMN1 gene, a most likely combination, of a plurality of possible combinations each comprising a possible copy number of the SMN1 gene and a possible copy number of the SMN2 gene summed to the copy number of any intact SMN gene(s) determined, given (a) a number of sequence reads (e.g., a unnormalized or normalized number of sequence reads) of the plurality of sequence reads with bases that support the SMN1 gene-specific base and (b) a number of sequence reads (e.g., a unnormalized or normalized number of sequence reads) of the plurality of sequence reads with bases that support a SMN2 gene-specific base of the SMN2 gene corresponding to the SMN1 gene-specific base. A possible copy number of the SMN1 gene can be or be about, for example, 1, 2, 3, 4, 5, 6, 7, 8, 9, 10, or more. A possible copy number of the SMN2 gene can be or be about, for example, 1, 2, 3, 4, 5, 6, 7, 8, 9, 10, or more.

In some embodiments, the most likely combination of the possible copy number of the SMN1 gene and the possible copy number of the SMN2 gene is associated with a highest posterior probability, relative to other combinations of the plurality of combinations given (a) the number of sequence reads of the plurality of sequence reads with bases that support the SMN1 gene-specific base and (b) the number of sequence reads of the plurality of sequence reads with bases that support the corresponding SMN2 gene-specific base. The highest posterior probability (or any probability of the present disclosure) can be or be about, for example, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more. The difference in the posterior probability (or any probability of the present disclosure) can be or be about, for example, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, or more.

In some embodiments, to determine the most likely combination of the possible copy number of the SMN1 gene and the possible combination of the SMN2 gene, the computing system can determine the most likely combination, of the plurality of possible combinations each comprising a possible copy number of the SMN1 gene and a possible copy number of the SMN2 gene summed to the copy number of any intact SMN genes determined, given a ratio of (a) a number of sequence reads of the plurality of sequence reads with bases that support the SMN1 gene-specific base and (b) a number of sequence reads of the plurality of sequence reads with bases that support the SMN2 gene-specific base of the SMN2 gene corresponding to the SMN1 gene-specific base. To determine the most likely combination of the possible copy number of the SMN1 gene and the possible combination of the SMN2 gene, the computing system can determine (a) a number of sequence reads of the plurality of sequence reads with bases that support the SMN1 gene-specific base and (b) a number of sequence reads of the plurality of sequence reads with bases that support the SMN2 gene-specific base of the SMN2 gene corresponding to the SMN1 gene-specific base. The computing system can determine the ratio of (a) a number of sequence reads of the plurality of sequence reads with bases that support the SMN1 gene-specific base and (b) a number of sequence reads of the plurality of sequence reads with bases that support the SMN2 gene-specific base of the SMN2 gene corresponding to the SMN1 gene-specific base. The computing system can determine the most likely combination, of the plurality of possible combinations each comprising a possible copy number of the SMN1 gene and a possible copy number of the SMN2 gene summed to the copy number of any intact SMN genes determined based on the ratio of (a) a number of sequence reads of the plurality of sequence reads with bases that support the SMN1 gene-specific base and (b) a number of sequence reads of the plurality of sequence reads with bases that support the SMN2 gene-specific base of the SMN2 gene corresponding to the SMN1 gene-specific base.

In some embodiments, to determine the most likely combination of the possible copy number of the SMN1 gene and the possible combination of the SMN2 gene, the computing system determines, for each of the plurality of SMN1 gene-specific bases, a most likely combination, of a plurality of possible combinations each comprising a possible copy number of the SMN1 gene and a possible copy number of the SMN2 gene summed to the copy number of any intact SMN genes determined, associated with a highest posterior probability given (a) a number of sequence reads of the plurality of sequence reads with bases that support the SMN1 gene-specific base and (b) a number of sequence reads of the plurality of sequence reads with bases that support a SMN2 gene-specific base of the SMN2 gene corresponding to the SMN1 gene-specific base. The number of sequence reads aligned to the SMN1 gene-specific base (or the SMN2 gene-specific base) can be or be about, for example, 3, 4, 5, 6, 7, 8, 9, 10, 20, 30, 40, 50, 60, 70, 80, 90, 100, or more. To determining the copy number of the SMN1 gene, the computing system can determine the copy number of the SMN1 gene based on the possible copy number of the SMN1 gene of the most likely combination of the possible copy number of the SMN1 gene and the possible copy number of the SMN2 gene determined for each of the plurality of SMN1 gene-specific bases.

In some embodiments, the SMN1 gene-specific base is a splicing enhancer. The SMN1 gene-specific base can be a base at c.840 of the SMN1 gene. In some embodiments, the SMN1 gene-specific base has a concordance with each of the plurality of SMN1 gene-specific bases other than the SMN1 gene-specific base above a predetermined concordance threshold. The predetermined concordance threshold (or any threshold of the present disclosure) can be or be about, for example, 80%, 81%, 82%, 83%, 84%, 85%, 86%, 87%, 88%, 89%, 90%, 91%, 92%, 93%, 94%, 95%, 96%, 97%, 98%, 99%, or more. For example, the concordance threshold can be 97%. The plurality of SMN1 gene-specific bases can comprise or comprise about, 4, 5, 6, 7, 8, 9, 10, 11, 12, 13, 14, 15, 16, 17, 18, 19, 20, 21, or more SMN1 gene-specific bases. For example, the plurality of SMN1 gene-specific bases can comprise 8 SMN1 gene-specific bases. Each of the plurality of SMN1 gene-specific bases can be on intron 6, the exon 7, intron 7, or the exon 8 of the SMN1 gene.

The plurality of SMN1 gene-specific bases if the subject is of a first race (or ethnicity), the plurality of SMN1 gene-specific bases if the subject is of a second race (or ethnicity), and the plurality of SMN1 gene-specific bases if the subject is of an unknown race can be different. A race can be, for example, Caucasian, African, African American, American Indian, Alaska Native, Asian, South Asian, East Asian, Native Hawaiian, Pacific Islander, or a combination thereof. The race (or ethnicity) of the subject can be unknown, and the plurality of SMN1 gene-specific bases can be race non-specific (or ethnicity non-specific). A race (or ethnicity) of the subject can be known, and the plurality of SMN1 gene-specific bases can specific to the race (or ethnicity) of the subject. In some embodiments, the computing system can receive race (or ethnicity) information of the subject. The computing system can select the plurality of SMN1 gene-specific bases from pluralities of SMN1 gene-specific bases based on the race (or ethnicity) information received.

The method 800 proceeds from block 820 to block 824, where the computing system determines a copy number of the SMN1 gene using the most likely combination of the possible copy number of the SMN1 gene and the possible copy number of the SMN2 gene determined for the SMN1 gene-specific base. Alternatively or additionally, the computing system determines a copy number of the SMN2 gene using the most likely combination of the possible copy number of the SMN1 gene and the possible copy number of the SMN2 gene determined for the SMN1 gene-specific base.

In some embodiments, to determine the copy number of the SMN1 gene, the computing system can determine the copy number of the SMN1 gene and a copy number of the SMN2 gene using the most likely combination of the possible copy number of the SMN1 gene and the possible copy number of the SMN2 gene determined for each of the plurality of SMN1 gene-specific bases. To determining the copy number, the computing system can determine the copy number of the SMN1 gene using the most likely combination of the possible copy number of the SMN1 gene and the possible copy number of the SMN2 gene determined for the SMN1 gene-specific base and a second predetermined posterior probability threshold for the combination of the possible copy number of SMN1 gene and the possible copy number of the SMN2 gene. The second predetermined posterior probability threshold (or any predetermined posterior probability threshold of the present disclosure) can be or be about, for example, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, or more. For example, the second predetermined posterior probability threshold can be 0.6 or 0.8.

In some embodiments a majority of the possible copy numbers of the SMN1 gene determined agree. The copy number of the SMN1 gene determined can be the agreed possible copy number of the SMN1 gene. The computing system can determine a possible combination comprising a possible copy number of the SMN1 gene and a possible copy number of the SMN2 gene summed to the copy number of any intact SMN genes determined given (a) a number of sequence reads of the plurality of sequence reads with bases that support any of the plurality of SMN1 gene-specific bases and (b) a number of sequence reads of the plurality of sequence reads with bases that support any of the plurality of corresponding SMN2 gene-specific bases. The computing system can determine the possible copy number of the possible combination is the agreed possible copy number of the SMN1 gene.

In some embodiments, to determine the copy number of the SMN1 gene, the computing system can determine the copy number of the SMN1 gene to be zero, one, or more than one. In some embodiments, the computing system can determine a spinal muscular atrophy (SMA) status of the subject based on the copy number of the SMN1 gene. The SMA status of the subject can comprise SMA, SMA carrier/not SMA, and not SMA carrier. In some embodiments, the computing system can determine the subject is a silent SMA carrier using a number of sequence reads of the plurality of sequence reads aligned to g.27134 of the SMN1 gene and the bases of the sequence reads aligned to the g.27134 of the SMN1 gene.

For example, the computing system at block 820 can identify the ratio of SMN1 to SMN2 reads overlapping locations where the genes have different base sequences. For positions where SMN1 differs from SMN2, the computing system can extract the reads that overlap either base on SMN1 or SMN2. From these reads, the computing system can count up the number of SMN1-specific bases and the number of SMN2-specific bases. The computing system can determine the fraction of SMN1 or SMN2 reads. The computing system can calculate the CN for the SMN1 and SMN2 at the positions where SMN1 is different from SMN2. The computing system can combine the full-length CN with the ratio of SMN1 to SMN2 to call the CN of SMN1 and the CN of SMN2. The computing system at block 824 can combine the CN from multiple fixed differences between SMN1 and SMN2 to get an accurate CN of SMN1 and an accurate CN of SMN2.

To call SMA/not SMA or carrier/not carrier. In some embodiments, the computing system can determine a copy number of truncated SMN gene(s) using (i) the copy number of the total SMN gene(s) determined and (ii) the copy number of the intact SMN gene(s) determined. The copy number of the truncated SMN gene(s) can be a difference of (i) the copy number of the total SMN gene(s) determined and (ii) the copy number of the intact SMN gene(s) determined.

Treatment. In some embodiments, the computing system can determine a treatment recommendation for the subject based on the copy number of the SMN1 gene determined. The treatment recommendation can comprise administering Nusinersen and/or Zolgensma to the subject.

The method 800 ends at block 828.

### Genotyping Cytochrome P450 Family 2 Subfamily D Member 6 Gene Using Sequencing Data

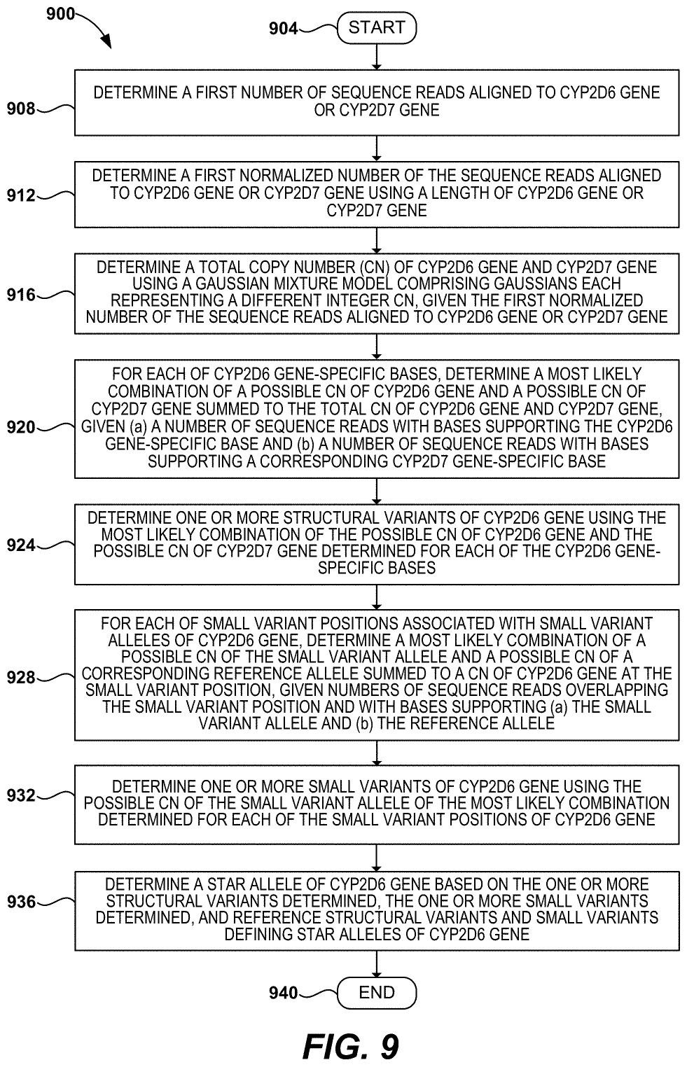

FIG. 9 is a flow diagram showing an exemplary method 900 of genotyping cytochrome P450 family 2 subfamily D member 6 gene using sequencing data, such as whole genome sequencing data. The method 900 may be embodied in a set of executable program instructions stored on a computer-readable medium, such as one or more disk drives, of a computing system. For example, the computing system 1100 shown in FIG. 11 and described in greater detail below can execute a set of executable program instructions to implement the method 900. When the method 900 is initiated, the executable program instructions can be loaded into memory, such as RAM, and executed by one or more processors of the computing system 1100. Although the method 900 is described with respect to the computing system 1100 shown in FIG. 11, the description is illustrative only and is not intended to be limiting. In some embodiments, the method 900 or portions thereof may be performed serially or in parallel by multiple computing systems.

The numbers of sequence reads (e.g., a unnormalized or normalized number of sequence reads) aligned to CYP2D6 gene or CYP2D7 gene can be used to determine the total copy number (CN) of the CYP2D6 gene and the CYP2D7 gene using a Gaussian mixture model. The total CN of the CYP2D6 gene and the CYP2D7 gene can be used to determine the CNs of CYP2D6 at various CYP2D6/CYP2D7 differentiating bases (also referred to herein as CYP2D6 gene-specific bases) by iterating through all possible combinations of CYP2D6 CNs and CYP2D7 CNs at CYP2D6/CYP2D7 differentiating bases. The CYP2D6 CNs at various CYP2D6/CYP2D7 differentiating bases can be used to call structural variants. For example, at each CYP2D6/CYP2D7 differentiating bases (also referred to herein as CYP2D6 gene-specific bases), the number of chromosomes carrying the CYP2D6 gene and the number of chromosomes carrying the CYP2D7 gene can be called by combining the total CN of the CYP2D6 gene and CYP2D7 gene with the read counts supporting each of the gene-specific bases. Based on the called total CN, all possible combinations of CYP2D6 CNs and CYP2D7 CNs can be iterated through to derive the combination that produces the highest posterior probability for the observed number of CYP2D6- and CYP2D7-supporting reads. Structural variants can be called by identifying the bases where the CN of the CYP2D6 gene changes.

One or more small variants can be determined. A small variants can be determined by, for each small variant position of the small variant, iterating through all possible combinations of the variant allele CN and the reference (non-variant) allele CN to determine the most likely variant allele CN using the sequence reads overlapping the small variant position in the CYP2D6 or CYP2D7 gene. For example, if there are three copies of the CYP2D6 gene in total and there are 10 reads supporting the variant allele and 20 reads supporting the reference allele, then the variant allele CN can be determined to be one, i.e., there is one copy of the CYP2D6 gene carrying the small variant. For example, small variants that define star alleles in sequence data (e.g., in a BAM file) can be searched. Small variants of interest can be divided into those that fall into CYP2D6/CYP2D7 homology regions and those that do not. For the former, variant reads aligned to the CYP2D6 gene or the CYP2D7 gene and overlap each small variant position of the CYP2D6 gene of interest or a corresponding position in the CYP2D7 gene can be searched. For the latter, reads aligned to the CYP2D6 gene and overlap a small variant position of the CYP2D6 gene of interest can be searched. CNs called in the region can also be take into account during small variant calling. The called structural variants and small variants can be matched against the definition of star alleles to call star alleles, which can be further grouped into haplotypes.

After the method 900 begins at block 904, the method 900 proceeds to block 908, where a computing system (e.g., the computing system 1100 described with references to FIG. 11) determines (i) a first number of sequence reads of a plurality of sequence reads aligned to cytochrome P450 family 2 subfamily D member 6 (CYP2D6) gene or cytochrome P450 Family 2 Subfamily D Member 7 (CYP2D7) gene. The first number of the sequence reads aligned to the first the CYP2D6 gene or the CYP2D7 gene (or any gene of the present disclosure) can be or be about, for example, 5, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, or more).

The computing system can receive sequence data comprising the plurality of sequence reads obtained from a sample of a subject aligned to the CYP2D6 gene or the CYP2D7 gene. In some embodiments, the sequencing data comprises whole genome sequencing (WGS) data or short-read WGS data. The subject can be a fetal subject, a neonatal subject, a pediatric subject, an adolescent subject, or an adult subject. The sample can comprise cells or cell-free DNA. The sample can comprise cells or cell-free DNA.

In some embodiments, a sequence read of the plurality of sequence reads is aligned to the CYP2D6 gene or the CYP2D7 gene with an alignment quality score of about zero. The alignment quality can be or be about, for example, 0, 0.01, 0.02, 0.03, 0.04, 0.05, 0.06, 0.07, 0.08, 0.09, 0.10, or more (on a scale from 0 to 1 from the alignment score).

In some embodiments, to determine (i) the first number of sequence reads of the plurality of sequence reads aligned to the CYP2D6 gene or the CYP2D7 gene, the computing system can determine (i) the first number of sequence reads of the plurality of sequence reads aligned to at least one exon or intron of the CYP2D6 gene (e.g., one of exons 1-9 or one of introns 1-8 of the CYP2D6 gene) and/or at least one of exon or intron of the CYP2D7 gene (e.g., one of exons 1-9 or one of introns 1-8 of the CYP2D7 gene).

The method 900 proceeds from block 908 to block 912, where the computing system determines (i) a first normalized number of the sequence reads aligned to the CYP2D6 gene or the CYP2D7 gene using (i) a length of the CYP2D6 gene or the CYP2D7 gene, respectively. The first normalized number of the sequence reads aligned to the CYP2D6 gene or the CYP2D7 gene (or any gene of the present disclosure)) can be or be about, for example, 1, 2, 3, 4, 5, 6, 7, 9, 10, 20, 30, 40, 50, 60, 70, 80, 90, 100, or more. The length of the CYP2D6 gene can be or be about, for example, 4.4 kb. The length of the CYP2D7 gene can be or be about, for example, 4.9 kb.

In some embodiments, to determine (i) the first normalized number of the sequence reads aligned to the CYP2D6 gene or the CYP2D7 gene, the computing system can determine (i) the first normalized number of the sequence reads aligned to the CYP2D6 gene or the CYP2D7 gene using (i) the length of the CYP2D6 gene or the CYP2D7 gene, respectively, and (iii) a depth of sequence reads of a region of a genome of the subject other than genetic loci comprising the CYP2D6 gene and the CYP2D7 gene in the sequence data. The depth of sequence reads of the region of the genome of the subject other than the genetic loci comprising the CYP2D6 gene and the CYP2D7 gene (or any genes of the present disclosure) in the sequence data can be or be about, for example, 3, 4, 5, 10, 20, 30, 40, 50, 60, 70, 80, 90, 100 or more.

To determine (i) the first normalized number of the sequence reads aligned to the CYP2D6 gene or the CYP2D7 gene and (ii) the second normalized number of the sequence reads aligned to the second region, the computing system can determine (i) a first CYP2D6 gene or the CYP2D7 gene-length normalized number of the sequence reads aligned to the CYP2D6 gene or the CYP2D7 gene using (i) the length of the CYP2D6 gene or the CYP2D7 gene, respectively. The computing system can determine (i) a first normalized depth of the sequence reads aligned to the CYP2D6 gene or the CYP2D7 gene from (i) the CYP2D6 gene or the CYP2D7 gene-length normalized number using the depth of the sequence reads of the region of the genome of the subject other than genetic loci comprising the CYP2D6 gene and the CYP2D7. The first normalized depth of the sequence reads aligned to the CYP2D6 gene or the CYP2D7 gene can be the first normalized number of the sequence reads aligned to the CYP2D6 gene or CYP2D7 gene, respectively.

In some embodiments, to determine (i) the first normalized number of the sequence reads aligned to the CYP2D6 gene or the CYP2D7 gene, the computing system can determine (i) the first normalized number of the sequence reads aligned to the CYP2D6 gene or the CYP2D7 gene using (i) a GC content of the CYP2D6 gene or the CYP2D7 gene and (iii) a depth of sequence reads of a region of a genome of the subject other than genetic loci comprising the CYP2D6 gene and the CYP2D7 gene in the sequence data, and (iv) a GC content of the region of the genome. The GC content of the CYP2D6 gene or the CYP2D7 gene (or any gene of the present disclosure) can be or be about, for example, 40%, 41%, 42%, 43%, 44%, 45%, 46%, 47%, 48%, 49%, 50%, 51%, 52%, 53%, 54%, 55%, 56%, 57%, 58%, 59%, or 60%. The depth of the sequence reads of the region of the genome of the subject other than the genetic loci comprising the CYP2D6 gene and the CYP2D7 gene in the sequence data can be or be about, for example, 3, 4, 5, 10, 20, 30, 40, 50, 100 or more. The GC content of the region of the genome of the subject other than the genetic loci comprising the CYP2D6 gene and the CYP2D7 gene (or any genes of the present disclosure) in the sequence data can be or be about, for example, 40%, 41%, 42%, 43%, 44%, 45%, 46%, 47%, 48%, 49%, 50%, 51%, 52%, 53%, 54%, 55%, 56%, 57%, 58%, 59%, or 60%.

The depth of the region can comprise an average depth of the sequence reads of the region of the genome of the subject other than the genetic loci comprising the CYP2D6 gene and the CYP2D7 gene in the sequencing data. The depth of the region can comprise a median depth of the sequence reads of the region of the genome of the subject other than the genetic loci comprising the CYP2D6 gene and the CYP2D7 gene in the sequencing data. The depth of the region can be or be about, 3, 4, 5, 6, 7, 8, 9, 10, 20, 30, 40, 50, 60, 70, 80, 90, 100, or more. The region can comprise about 500, 1000, 1500, 2000, 2500, 3000, 3500, 4000, or more pre-selected regions of about 0.5 kb, 1 kb, 1.5 kb, 2 kb, 2.5 kb, or 3 kb, in length each across the genome of the subject. For example, the region can comprise about 3000 pre-selected regions of about 2 kb in length each across the genome of the subject.

In some embodiments, (i) the first normalized number of the sequence reads aligned to the CYP2D6 gene or the CYP2D7 gene and/or (ii) the second normalized number of the sequence reads aligned to the second region is or is about 10, 20, 30, 40, 50, 60, 70, 80, 90, 100, or more. For example, (i) the first normalized number of the sequence reads aligned to the CYP2D6 gene or the CYP2D7 gene and/or (ii) the second normalized number of the sequence reads aligned to the second region is about 30 to about 40.

The method 900 proceeds from block 912 to block 916, where the computing system determines (i) a total copy number of the CYP2D6 gene and the CYP2D7 gene using a Gaussian mixture model comprising a plurality of Gaussians each representing a different integer copy number, given (i) the first normalized number of the sequence reads aligned to the CYP2D6 gene or the CYP2D7 gene. The total copy number of the CYP2D6 gene and the CYP2D7 gene (or any genes of the present disclosure) can be or be about, 1, 2, 3, 4, 5, 6, 7, 8, 9, 10, or more.

In some embodiments, the Gaussian mixture model comprises a one-dimensional Gaussian mixture model. The plurality of Gaussians of the Gaussian mixture model can represent integer copy numbers, for example, 0 to 5, 0 to 6, 0 to 7, 0 to 8, 0 to 9, 0 to 10, 0 to 11, 0 to 12, 0 to 13, 0 to 14, or 0 to 15. For example, the plurality of Gaussians of the Gaussian mixture model can represent integer copy numbers 0 to 10. A mean (e.g., 1, 2, 3, 4, 5, 6, 7, 8, 9, 10, or more) of each of the plurality of Gaussians can be the integer copy number represented by the Gaussian (e.g., copy numbers of 1, 2, 3, 4, 5, 6, 7, 8, 9, 10, or more). The standard deviation of a Gaussian can be or be about, for example, 0.1, 0.2, 0.3, 0.4, 0.5, 0.6, 0.7, 0.8, 0.9, 1, or more.

In some embodiments, to determine (i) the total copy number of the CYP2D6 gene and the CYP2D7 gene, the computing system can determine (i) the total copy number of the CYP2D6 gene and the CYP2D7 gene using the Gaussian mixture model and a first predetermined posterior probability threshold, given (i) the first normalized number of the sequence reads aligned to the CYP2D6 gene or the CYP2D7 gene. The first predetermined posterior probability threshold (or any predetermined posterior probability threshold of the present disclosure) can be or be about, for example, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, or more. For example, the first predetermined posterior probability threshold can be 0.95.

The method 900 proceeds from block 916 to block 920, where the computing system determines, for one of a plurality of CYP2D6 gene-specific bases (also referred to herein as CYP2D6/CYP2D7 differentiating bases), a most likely combination, of a plurality of possible combinations each comprising a possible copy number of the CYP2D6 gene and a possible copy number of the CYP2D7 gene summed to the total copy number of the CYP2D6 gene and the CYP2D7 gene determined, given (a) a number of sequence reads (e.g., a unnormalized or normalized number of sequence reads) of the plurality of sequence reads with bases that support the CYP2D6 gene-specific base and (b) a number of sequence reads (e.g., a unnormalized or normalized number of sequence reads) of the plurality of sequence reads with bases that support a CYP2D7 gene-specific base of corresponding to the CYP2D6 gene-specific base. A possible copy number of the CYP2D6 gene can be or be about, for example, 1, 2, 3, 4, 5, 6, 7, 8, 9, 10, or more. A possible copy number of the CYP2D7 gene can be or be about, for example, 1, 2, 3, 4, 5, 6, 7, 8, 9, 10, or more.

In some embodiments, the most likely combination of the possible copy number of the CYP2D6 gene and the possible copy number of the CYP2D7 gene is associated with a highest posterior probability, relative to other combinations of the plurality of combinations given (a) the number of sequence reads of the plurality of sequence reads with bases that support the CYP2D6 gene-specific base and (b) the number of sequence reads of the plurality of sequence reads with bases that support the corresponding CYP2D7 gene-specific base. The highest posterior probability (or any probability of the present disclosure) can be or be about, for example, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more. The difference in the posterior probability (or any probability of the present disclosure) can be or be about, for example, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, or more.

In some embodiments, to determine the most likely combination comprising a possible copy number of the CYP2D6 gene and a possible copy number, the computing system can determine the most likely combination, of the plurality of possible combinations each comprising a possible copy number of the CYP2D6 gene and a possible copy number of the CYP2D7 gene summed to the total copy number of the CYP2D6 gene and the CYP2D7 gene determined, given a ratio of (a) the number of sequence reads of the plurality of sequence reads with bases that support the CYP2D6 gene-specific base and (b) the number of sequence reads of the plurality of sequence reads with bases that support a CYP2D7 gene-specific base of corresponding to the CYP2D6 gene-specific base. To determine the most likely combination comprising the possible copy number of the CYP2D6 gene and the possible copy number, the computing system can determine (a) the number of sequence reads of the plurality of sequence reads with bases that support the CYP2D6 gene-specific base and (b) the number of sequence reads of the plurality of sequence reads with bases that support a CYP2D7 gene-specific base of corresponding to the CYP2D6 gene-specific base. The computing system can determine a ratio of (a) the number of sequence reads of the plurality of sequence reads with bases that support the CYP2D6 gene-specific base and (b) the number of sequence reads of the plurality of sequence reads with bases that support a CYP2D7 gene-specific base of corresponding to the CYP2D6 gene-specific base. The computing system can determine the most likely combination, of the plurality of possible combinations each comprising a possible copy number of the CYP2D6 gene and a possible copy number of the CYP2D7 gene summed to the total copy number of the CYP2D6 gene and the CYP2D7 gene determined, given the ratio (a) a number of sequence reads of the plurality of sequence reads with bases that support the CYP2D6 gene-specific base and (b) a number of sequence reads of the plurality of sequence reads with bases that support a CYP2D7 gene-specific base of corresponding to the CYP2D6 gene-specific base.

In some embodiments, to determine the most likely combination of the possible copy number of the CYP2D6 gene and the possible combination of the CYP2D7 gene, the computing system determines, for each of the plurality of CYP2D6 gene-specific bases, a most likely combination, of a plurality of possible combinations each comprising a possible copy number of the CYP2D6 gene and a possible copy number of the CYP2D7 gene summed to the total copy number the CYP2D6 gene and the CYP2D7 gene determined, associated with a highest posterior probability given (a) a number of sequence reads of the plurality of sequence reads with bases that support the CYP2D6 gene-specific base and (b) a number of sequence reads of the plurality of sequence reads with bases that support a CYP2D7 gene-specific base of the CYP2D7 gene corresponding to the CYP2D6 gene-specific base. The number of sequence reads aligned to the SMN1 gene-specific base (or the SMN2 gene-specific base) can be or be about, for example, 3, 4, 5, 6, 7, 8, 9, 10, 20, 30, 40, 50, 60, 70, 80, 90, 100, or more. To determine the allele of the CYP2D6 gene the subject has, the computing system can determine the allele of the CYP2D6 gene the subject has is the small variant or the structural variant of the CYP2D6 gene, or neither, using the most likely combination of the possible copy number of the CYP2D6 gene and the possible copy number of the CYP2D7 gene determined for each of the plurality of CYP2D6 gene-specific bases.

In some embodiments, the CYP2D6 gene-specific base has a concordance with each of the plurality of CYP2D6 gene-specific bases other than the CYP2D6 gene-specific base above a predetermined concordance threshold. The concordance threshold (or any threshold of the present disclosure) can be or be about, for example, 80%, 81%, 82%, 83%, 84%, 85%, 86%, 87%, 88%, 89%, 90%, 91%, 92%, 93%, 94%, 95%, 96%, 97%, 98%, 99%, or more. For example, the predetermined concordance threshold can be 97%. The plurality of CYP2D6 gene-specific bases can comprise or comprise about, for example, 10, 20, 30, 40, 50, 60, 70, 80, 90, 100, 110, 118, 120, 130, 140, 150, 160, 170, or more, CYP2D6 gene-specific bases. For example, the plurality of CYP2D6 gene-specific bases can comprise 118 CYP2D6 gene-specific bases.

The method 900 proceeds from block 920 to block 924, where the computing system determines one or more structural variants of the CYP2D6 gene the subject has using the most likely combination of the possible copy number of the CYP2D6 gene and the possible copy number of the CYP2D7 gene determined for the CYP2D6 gene-specific base. For example, the computing system can identify the ratio of CYP2D6 to CYP2D7 reads overlapping locations where the genes have different base sequences. For positions where CYP2D6 differs from CYP2D7, the computing system can extract the reads that overlap either base on CYP2D6 or CYP2D7. From these reads, the computing system can count up the number of CYP2D6-specific bases and the number of CYP2D7-specific bases. The computing system can determine the fraction of CYP2D6 or CYP2D7 reads. The computing system can calculate the CN for CYP2D6 and CYP2D7 at the positions where CYP2D6 is different from CYP2D7. The computing system can combine the total CN of CYP2D6 and CYP2D7 with the ratio of CYP2D6 to CYP2D7 to call the CN of CYP2D6 and the CN of CYP2D7. The computing system can make small variant calls using the CNs of CYP2D6 and CYP2D7 at one or more fixed differences between CYP2D6 and CYP2D7. The computing system can make structural variant calls by combining CNs of CYP2D6 and CYP2D7 at multiple fixed differences between CYP2D6 and CYP2D7 to determine the presence of a transition between CNs at CYP2D6 and CYP2D7 that defines the type of structural variant that is in the sample.

REP-containing fusion genes. In some embodiments, the computing system can determine (ii) a second number of sequence reads of the plurality of sequence reads aligned to a spacer region between the CYP2D7 gene and a repetitive element REP7 downstream of the CYP2D7 gene. The second number of the sequence reads of the plurality of sequence reads aligned to the spacer region between the CYP2D7 gene and the repetitive element REP7 downstream of the CYP2D7 gene can be or be about, for example, 5, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, or more. The computing system can determine (ii) a second normalized number of the sequence reads aligned to the spacer region using (ii) a length of the spacer region. The second normalized number of the sequence reads aligned to the spacer region can be or be about, for example, 1, 2, 3, 4, 5, 6, 7, 9, 10, 20, 30, 40, 50, 60, 70, 80, 90, 100, or more. The length of the spacer region can be or be about, for example, 1.5 kb. The computing system can determine (ii) a copy number of the spacer region using the Gaussian mixture model given (ii) the second normalized number of the sequence reads aligned to the spacer region. The copy number of the spacer region can be or be about, for example, 1, 2, 3, 4, 5, 6, 7, 8, 9, 10, or more. To determine the allele of the CYP2D6 gene the subject has, the computing system can determine the allele of the CYP2D6 gene the subject has is the small variant or the structural variant of the CYP2D6 gene, or neither, using the combination of the possible copy number of the CYP2D6 gene and the possible copy number of the CYP2D7 gene determined for the CYP2D6 gene-specific base and the copy number of the spacer region. The structural variant can comprise a CYP2D6/CYP2D7 fusion allele with the spacer region and the repetitive element REP7 downstream of the CYP2D6/CYP2D7 fusion allele.

The method 900 proceeds from block 924 to block 928, where the computing system can, for a small variant position of the CYP2D6 gene associated with a small variant allele of the CYP2D6 gene, determine a most likely combination of a possible copy number of the small variant allele of the CYP2D6 gene at the small variant position and a possible copy number of a reference allele of the CYP2D6 gene at the small variant position summed to a copy number of the CYP2D6 gene at the small variant position, given (a) a number of sequence reads (e.g., a unnormalized or normalized number of sequence reads) aligned to the CYP2D6 gene that overlap the small variant position and with bases supporting the small variant allele of the CYP2D6 gene at the small variant position and (b) a number of sequence reads (e.g., a unnormalized or normalized number of sequence reads) aligned to the CYP2D6 gene that overlap the small variant position and with bases supporting the reference allele of the CYP2D6 gene at the small variant position. The possible copy number of the small variant allele of the CYP2D6 gene of the most likely combination at the small variant position can indicate the one or more small variants of the CYP2D6 gene

The computing system can determine, for each of a plurality of small variant positions of the CYP2D6 gene, the small variant position being associated with a small variant allele of the CYP2D6 gene, determine a most likely combination of a possible copy number of the small variant allele of the CYP2D6 gene at the small variant position and a possible copy number of a reference allele of the CYP2D6 gene at the small variant position summed to a copy number of the CYP2D6 gene at the small variant position, given (a) a number of sequence reads (e.g., a unnormalized or normalized number of sequence reads) aligned to the CYP2D6 gene that overlap the small variant position and with bases supporting the small variant allele of the CYP2D6 gene at the small variant position and (b) a number of sequence reads (e.g., a unnormalized or normalized number of sequence reads) aligned to the CYP2D6 gene that overlap the small variant position and with bases supporting the reference allele of the CYP2D6 gene at the small variant position. The possible copy numbers of the small variant alleles of the CYP2D6 gene of the most likely combinations at the plurality of small variant positions can indicate the one or more small variants of the CYP2D6 gene.

In some embodiments, the computing system can determine the copy number of the CYP2D6 gene at the small variant position. The copy number of the CYP2D6 gene at the small variant position can comprise a copy number of the CYP2D6 gene. The copy number of the CYP2D6 gene at the small variant position can comprise a copy number of the CYP2D6 gene of possible copy numbers of the CYP2D6 gene of the most likely combinations determined. The copy number of the CYP2D6 gene at the small variant position can comprise a copy number of the CYP2D6 gene of possible copy numbers of the CYP2D6 gene of the most likely combinations determined and closest to the small variant position. The copy number of the CYP2D6 gene at the small variant position can comprise a copy number of the CYP2D6 gene at a 5′ position or 3′ position of the small variant position.

In some embodiments, the computing system can (a) determine the number of sequence reads (e.g., a unnormalized or normalized number of sequence reads) with bases supporting the small variant allele of the CYP2D6 gene. The computing system can (b) determine the number of sequence reads (e.g., a unnormalized or normalized number of sequence reads) with bases supporting the reference allele of the CYP2D6 gene.

The method 900 proceeds from block 928 to block 932, where the computing system determines one or more small variants the CYP2D6 gene using the possible copy number of the small variant allele of the CYP2D6 gene of the most likely combination determined. The computing system can determine one or more small variants the CYP2D6 gene using the possible copy numbers of the small variant alleles of the CYP2D6 gene of the most likely combinations at the plurality of small variant positions determined

In some embodiments, the small variant position is in a CYP2D6/CYP2D7 homology region. To determine the most likely combination, the computing system can determine the most likely combination of the possible copy number of the small variant allele of the CYP2D6 gene at the small variant position and the possible copy number of the reference allele of the CYP2D6 gene at the small variant position summed to the copy number of the CYP2D6 gene at the small variant position given (a) a number of sequence reads aligned to the CYP2D6 gene or CYP2D7 gene with bases supporting the small variant allele of the CYP2D6 gene at the small variant position and/or (b) a number of sequence reads aligned to the CYP2D6 gene or CYP2D7 gene with bases supporting the reference allele of the CYP2D6 at the small variant position. In some embodiments, the small variant position is not in a CYP2D6/CYP2D7 homology region. To determine the most likely combination, the computing system can determine the most likely combination of the possible copy number of the small variant allele of the CYP2D6 gene at the small variant position and the possible copy number of the reference allele of the CYP2D6 gene at the small variant position summed to the copy number of the CYP2D6 gene at the small variant position given (a) a number of sequence reads aligned to the CYP2D6 gene (and not to the CYP2D7 gene) with bases supporting the small variant allele of the CYP2D6 gene at the small variant position and/or (b) a number of sequence reads aligned to the CYP2D6 gene (and not CYP2D7 gene) with bases supporting the reference allele of the CYP2D6 at the small variant position.

For example, the computing system can first determine the SV (structural variant, e.g. deletion or duplication) pattern and breakpoints based on the CN of paralog-specific bases. Additionally or alternatively, the computing system can then call a pre-defined set of small variants (these are variants specific to the gene of interest, e.g. CYP2D6, and are a different set from the paralog-differentiating bases) based on the read alignments, the total CN, and (sometimes) the SV pattern and breakpoints determined at the first step. Since alignments are not always accurate, the computing system can extract out the bases of interest in reads that align to either paralog.

The method 900 proceeds from block 932 to block 936, where the computing system can determine a star allele and/or a haplotype of the CYP2D6 gene the subject has using the one or more structural variants of the CYP2D6 gene determined and/or the one or more small variants of the CYP2D6 gene determined. The star allele can be associated with a known function. A star allele and/or a haplotype of the CYP2D6 gene can comprise, for example, CYP2D6*1, *2, *3, *4, *5, *6, *7, *9, *10, *11, *13, *14, *15, *17, *21, *22, *28, *29, *31, *33, *34, *35, *36, *37, *38, *39, *40, *41, *43, *45, *46, *47, *49, *52, *54, *56, *57, *59, *64, *65, *68, *71, *72, *82, *84, *86, *94, *95, *99, *100, *101, *106, *108, *111, *112, *113, or a combination thereof.

Enzymatic activity. In some embodiments, the computing system can determine a level of CYP2D6 enzymatic activity in the subject using the allele of the CYP2D6 gene determined. The enzymatic activity can be poor, intermediate, normal, or ultrarapid. The computing system can determine a dosage recommendation of a treatment and/or a treatment recommendation for the subject based on the one or more the small variants and/or the one or more structural variants.

The method 900 ends at block 940.

### Paralog Genotyping Using Sequencing Data

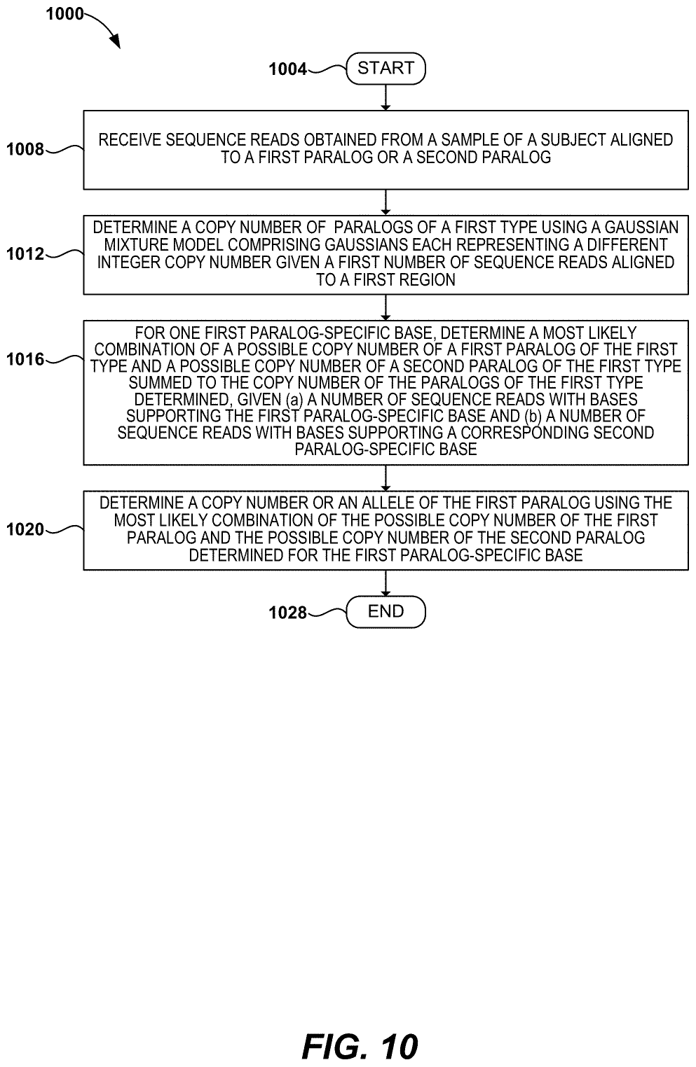

FIG. 10 is a flow diagram showing an exemplary method 1000 of paralog genotyping using sequencing data, such as whole genome sequencing data. The method 1000 may be embodied in a set of executable program instructions stored on a computer-readable medium, such as one or more disk drives, of a computing system. For example, the computing system 1100 shown in FIG. 11 and described in greater detail below can execute a set of executable program instructions to implement the method 1000. When the method 1000 is initiated, the executable program instructions can be loaded into memory, such as RAM, and executed by one or more processors of the computing system 1100. Although the method 1000 is described with respect to the computing system 1100 shown in FIG. 11, the description is illustrative only and is not intended to be limiting. In some embodiments, the method 1000 or portions thereof may be performed serially or in parallel by multiple computing systems.

After the method 1000 begins at block 1004, the method 1000 proceeds to block 1008, where a computing system (e.g., the computing system 1100 described with references to FIG. 11) receives sequence data comprising a plurality of sequence reads obtained from a sample of a subject aligned to a first paralog or a second paralog. Techniques for generating sequence reads include sequencing by synthesis using, for example, MINISEQ, MISEQ, NEXTSEQ, HISEQ, and NOVASEQ sequencing instruments from Illumina, Inc. (San Diego, Calif.).

The method 1000 proceeds from block 1008 to block 1012, where the computing system determines a copy number of paralogs of a first type using a Gaussian mixture model comprising a plurality of Gaussians each representing a different integer copy number given (i) a first number of sequence reads aligned to a first region. The copy number of the paralogs of the first type (or any type of the present disclosure) can be or be about, for example, 1, 2, 3, 4, 5, 6, 7, 8, 9, 10, or more.

The plurality of Gaussians of the Gaussian mixture model can represent integer copy numbers, for example, 0 to 5, 0 to 6, 0 to 7, 0 to 8, 0 to 9, 0 to 10, 0 to 11, 0 to 12, 0 to 13, 0 to 14, or 0 to 15. For example, the plurality of Gaussians of the Gaussian mixture model can represent integer copy numbers 0 to 10. A mean (e.g., 1, 2, 3, 4, 5, 6, 7, 8, 9, 10, or more) of each of the plurality of Gaussians can be the integer copy number represented by the Gaussian (e.g., copy numbers of 1, 2, 3, 4, 5, 6, 7, 8, 9, 10, or more). The standard deviation of a Gaussian can be or be about, for example, 0.1, 0.2, 0.3, 0.4, 0.5, 0.6, 0.7, 0.8, 0.9, 1, or more.

In some embodiments, the computing system can determine (i) a first number of sequence reads of the plurality of sequence reads in the sequence data obtained from the sample of the subject aligned to the first region. The first number of the sequence reads of the plurality of sequence reads in the sequence data obtained from the sample of the subject aligned to the first region (or any region of the disclosure) can be or be about, for example, 5, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, or more. The computing system can determine (i) a first normalized number of the sequence reads aligned to the first region using (i) a length of the first region. The first normalized number of the sequence reads aligned to the first region (or any region of the disclosure) can be or be about, for example, 1, 2, 3, 4, 5, 6, 7, 9, 10, 20, 30, 40, 50, 60, 70, 80, 90, 100, or more. The length of the first region can be or be about, for example, 1 kb, 2 kb, 3 kb, 4 kb, 5 kb, 6 kb, 7 kb, 8 kb, 9 kb, 10 kb, 11 kb, 12 kb, 13 kb, 14 kb, 15 kb, 16 kb, 17 kb, 18 kb, 19 kb, 20 kb, 21 kb, 22 kb, 23 kb, 24 kb, 25 kb, 26 kb, 27 kb, 28 kb, 29 kb, 30 kb, or more. To determine the copy number of the first type of paralogs, the computing system can determine the copy number of the first type of paralogs using the Gaussian mixture model given (i) the first normalized number of the sequence reads aligned to the first region.

In some embodiments, the computing system can determine a copy number of one or more paralogs of a second type using the Gaussian mixture given (ii) a second number of sequence reads aligned to a second region. The copy number of the one or more paralogs of the second type (or any type of the present disclosure) can be or be about, for example, 1, 2, 3, 4, 5, 6, 7, 8, 9, 10, or more. To determine the copy number or the allele of first paralog, the computing system can determine the copy number or the allele of the first paralog using the most likely combination of the possible copy number of the first paralog and the possible copy number of the second paralog determined for the first paralog-specific base and the copy number of the one or more paralogs of the second type. The computing system can determine a copy number of paralogs of a third type from the copy number of the paralogs of the first type and the copy number of the paralogs of the second type. The copy number of the paralogs of the third type (or any type of the present disclosure) can be or be about, for example, 1, 2, 3, 4, 5, 6, 7, 8, 9, 10, or more. To determine the copy number or the allele of the first paralog, the computing system can determine the copy number or the allele of the first paralog using the most likely combination of the possible copy number of the first paralog and the possible copy number of the second paralog determined for the first paralog-specific base.

Methods for aligning the sequence reads to a reference genome sequence can utilize aligners such as Burrows-Wheeler Aligner (BWA) and iSAAC. Other alignment methods include BarraCUDA, BFAST, BLASTN, BLAT, Bowtie, CASHX, Cloudburst, CUDA-EC, CUSHAW, CUSHAW2, CUSHAW2-GPU, drFAST, ELAND, ERNE, GNUMAP, GEM, GensearchNGS, GMAP and GSNAP, Geneious Assembler, LAST, MAQ, mrFAST and mrsFAST, MOM, MOSAIK, MPscan, Novoaligh & NovoalignCS, NextGENe, Omixon, PALMapper, Partek, PASS, PerM, PRIMEX, QPalma, RazerS, REAL, cREAL, RMAP, rNA, RT Investigator, Segemehl, SeqMap, Shrec, SHRiMP, SLIDER, SOAP, SOAP2, SOAP3 and SOAP3-dp, SOCS, SSAHA and SSAHA2, Stampy, SToRM, Subread and Subjunc, Taipan, UGENE, VelociMapper, XpressAlign, and ZOOM.

The method 1000 proceeds from block 1012 to block 1016, where the computing system determines, for one of a plurality of first paralog-specific bases, a most likely combination, of a plurality of possible combinations each comprising a possible copy number of a first paralog of the first type and a possible copy number of a second paralog of the first type summed to the copy number of the paralogs of the first type determined, given (a) a number of sequence reads (e.g., a unnormalized or normalized number of sequence reads) of the plurality of sequence reads with bases that support the first paralog-specific base and (b) a number of sequence reads (e.g., a unnormalized or normalized number of sequence reads) of the plurality of sequence reads with bases that support a second paralog-specific base of the second paralog corresponding to the first paralog-specific base.

The method 1000 proceeds from block 1016 to block 1020, where the computing system determines a copy number or an allele of the first paralog using the most likely combination of the possible copy number of the first paralog and the possible copy number of the second paralog determined for the first paralog-specific base.

In some embodiments, the first paralog is survival of motor neuron 1 (SMN1) gene. The second paralog can be survival of motor neuron 2 (SMN2) gene. The first region can comprise at least one exon 1 to exon 6 of the SMN1 gene and at least one exon 1 to exon 6 of the SMN2 gene. The second region can comprise at least one of exon 7 and exon 8 of the SMN1 gene and at least one of exon 7 and exon 8 of the SMN2 gene. The paralogs of the first type can comprise an intact SMN1 gene and an intact SMN2 gene. The one or more paralogs of the second type can comprise the intact SMN1 gene, the intact SMN2 gene, a truncated SMN1 gene, or a truncated SMN2 gene. The copy number of the first paralog can comprise a copy number of the SMN1 gene. The computing system can determine the copy number of the SMN1 gene implementing the method 800 (or a portion thereof) described with reference to FIG. 8.

In some embodiments, the first paralog is Cytochrome P450 Family 2 Subfamily D Member 6 (CYP2D6) gene. The second paralog can be Cytochrome P450 Family 2 Subfamily D Member 7 (CYP2D7) gene. The first region can comprise the CYP2D6 gene and the CYP2D7 gene. The second region can comprise a spacer region between the CYP2D7 gene and a repetitive element REP7 downstream of the CYP2D7 gene. The paralogs of the first type can comprise the CYP2D6 gene and the CYP2D7 gene. The one or more paralogs of the second type can comprise a CYP2D6/CYP2D7 fusion allele with the spacer region and the repetitive element REP7 downstream of the CYP2D6/CYP2D7 fusion allele. The allele of the first paralog can comprise an allele of the CYP2D6 gene the subject has is a small variant or a structural variant of the CYP2D6 gene. The computing system can determine the allele of the CYP2D6 gene implementing the method 900 (or a portion thereof) described with reference to FIG. 9.

The first and second paralogs can be different in different embodiments. Examples of the first and second paralogs include, but are not limited to, SMN1 gene and SMN2 gene; CYP2D6 gene and CYP2D7 gene; double homeobox 4 (DUX4) gene, DUX4c gene, DUX4 like 2 (DUX4L2) gene, DUX4 like 3 (DUX4L3) gene, DUX4 like 4 (DUX4L4) gene, DUX4 like 5 (DUX4L5) gene, DUX4 like 6 (DUX4L6) gene, DUX4 like 7 (DUX4L7) gene, and double homeobox 2 (DUX2) gene; and Ribosomal Protein S17 (RpS17) gene and RpS17-like (RpS17L) gene. In some embodiments, the computing system can determine the copy number or the allele of the first paralog implementing the method 800 (or a portion thereof) described with reference to FIG. 8 and/or the method 900 (or a portion thereof) described with reference to FIG. 9.

In some embodiments, the first paralog and the second paralog have a sequence identity of, or of about, 80%, 81%, 82%, 83%, 84%, 85%, 86%, 87%, 88%, 89%, 90%, 91%, 92%, 93%, 94%, 95%, 96%, 97%, 98%, 99%, or more. For example, the first paralog and the second paralog have a sequence identity of at least 90%.

The method 1000 ends at block 1024.

### Execution Environment

In FIG. 11 depicts a general architecture of an example computing device 1100 configured for paralog genotyping. The general architecture of the computing device 1100 depicted in FIG. 11 includes an arrangement of computer hardware and software components. The computing device 1100 may include many more (or fewer) elements than those shown in FIG. 11. It is not necessary, however, that all of these generally conventional elements be shown in order to provide an enabling disclosure. As illustrated, the computing device 1100 includes a processing unit 1110, a network interface 1120, a computer readable medium drive 1130, an input/output device interface 1140, a display 1150, and an input device 1160, all of which may communicate with one another by way of a communication bus. The network interface 1120 may provide connectivity to one or more networks or computing systems. The processing unit 1110 may thus receive information and instructions from other computing systems or services via a network. The processing unit 1110 may also communicate to and from memory 1170 and further provide output information for an optional display 1150 via the input/output device interface 1140. The input/output device interface 1140 may also accept input from the optional input device 1160, such as a keyboard, mouse, digital pen, microphone, touch screen, gesture recognition system, voice recognition system, gamepad, accelerometer, gyroscope, or other input device.

The memory 1170 may contain computer program instructions (grouped as modules or components in some embodiments) that the processing unit 1110 executes in order to implement one or more embodiments. The memory 1170 generally includes RAM, ROM and/or other persistent, auxiliary or non-transitory computer-readable media. The memory 1170 may store an operating system 1172 that provides computer program instructions for use by the processing unit 1110 in the general administration and operation of the computing device 1100. The memory 1170 may further include computer program instructions and other information for implementing aspects of the present disclosure.

For example, in one embodiment, the memory 1170 includes a paralog genotyping module 1174 for genotyping one or more paralogs using sequencing data, such as the method 1000 described with reference to FIG. 10. Alternatively or additionally, the paralog genotyping module 1174 can be, or can include, a module for determining SMN1 copy number using sequencing data, such as the method 800 described with reference to FIG. 8. Alternatively or additionally, the paralog genotyping module 1174 can be, or can include, a module for genotyping CYP2D6 gene using sequencing data, such as the method 900 described with reference to FIG. 9. In addition, the memory 1170 may include or communicate with the data store 1190 and/or one or more other data stores that store sequencing data and/or the results of genotyping one or more paralogs.

### EXAMPLES

Some aspects of the embodiments discussed above are disclosed in further detail in the following examples, which are not in any way intended to limit the scope of the present disclosure.

### Example 1

### Spinal Muscular Atrophy Diagnosis and Carrier Screening from Whole Genome Sequencing Data

Spinal muscular atrophy (SMA), caused by loss of the functional SMN1 gene but retention of the paralogous SMN2 gene, is a leading genetic cause of early childhood death. Due to the near identical sequences of SMN1 and its paralog SMN2, analysis of this region by next generation sequencing (NGS)-based pipelines has been challenging. Preconception population-wide SMA screening of potential parents to quantify the copy number (CN) of SMN1 is recommended by the American College of Medical Genetics.

This example describes a bioinformatics method that accurately identifies the CN of SMN1 and SMN2 using whole genome sequencing (WGS) data. The method calculates the CNs of SMN1 and SMN2 using read depth and eight informative reference genome differences between SMN1 and SMN2.

The SMN1/2 statuses in a total of 12,747 short-read whole genomes sequenced to high depths (>30×) across five ethnic populations were characterized. Across these samples, a total of 251 (1317) samples with whole gene losses (gains) of SMN1 and 6241 (374) samples with losses (gains) of SMN2 were identified. A pan-ethnic carrier frequency of 2% was calculated, consistent with previous studies. Additionally, the CN calls were validated and with all (48/48) SMN1 and 98% ( 47/48) of SMN2 CN calls agreeing with those measured with digital PCR.

This WGS-based SMN copy number calling method can be used to identify both carrier and affected status of SMA, enabling SMA testing to be offered as a comprehensive test in neonatal care and also an accurate screening tool for carrier status in large-scale WGS sequencing projects.

### Introduction

With recent advances in next-generation sequencing (NGS), it is now possible to profile a large number of genes or even the entire genome at high throughput and in a clinically relevant timeframe. Driven by these advances, many countries are undertaking large scale population sequencing efforts wherein testing for rare genetic disorders including carrier status will be one of the primary drivers. Spinal muscular atrophy (SMA), an autosomal recessive neuromuscular disorder characterized by loss of alpha motor neurons, causes severe muscle weakness and atrophy presenting at or shortly after birth. SMA is the leading genetic cause of infant death after cystic fibrosis. The incidence of SMA is 1 in 6000-10,000 live births, and the carrier frequency is 1:40-80 among different ethnic groups. The four clinical types of SMA are classified based on age of onset and severity of the disease: very weak infants unable to sit unsupported (Type I), weak sitters but unable to stand (Type II), ambulant patients with weaker legs than arms (Type III), and adult onset SMA that is fairly benign (Type IV). Early detection of SMA can be critical for long term quality of life due to the availability of two early treatments, Nusinersen and Zolgensma, which have received FDA approval for the amelioration of SMA.

The SMN region includes two paralogous genes: SMN1 and SMN2. SMN2 is 875 kb away from SMN1 on 5q and is caused by an ancestral gene duplication that is unique to the human lineage. The genomic region around SMN1/2 is subject to unequal crossing-over and gene conversion, resulting in variable copy numbers (CNs) of SMN1 and SMN2. SMN2 has greater than 99.9% sequence identity to SMN1 and one of the base differences, c.840C>T in exon 7, has a critical functional consequence. By interrupting a splicing enhancer, c.840T promotes skipping of exon 7, resulting in the vast majority of SMN2-derived transcripts (70-85%, depending on tissue) being unstable and not fully functional. Approximately 95% of SMA cases result from biallelic absence of the functional c.840C nucleotide caused by either a deletion of SMN1 or gene conversion to SMN2 (c.840T). In the remaining 5% of SMA cases, patients have other pathogenic variants in SMN1 in trans with an allele missing c.840C. SMN2 can produce a small amount of functional protein, and the number of SMN2 copies in an individual modifies the disease severity and is highly correlated with the clinical types described above.

Due to the high incidence rate and disease severity, population-wide SMA screening is recommended by the American College of Medical Genetics. The utility of population-wide carrier screening has been demonstrated in pilot studies. Screening for SMA includes: 1) determining the copy number of SMN1 for SMA diagnosis and carrier testing and 2) determining the copy number of SMN2 for clinical classification and prognosis. Traditionally, SMA testing and carrier testing are done with polymerase chain reaction (PCR) based assays, such as quantitative PCR (qPCR), multiplex ligation-dependent probe amplification (MLPA) and digital PCR. These methods primarily determine the copy number of SMN1 based on the c.840C>T site that differs between SMN1 and SMN2. This example demonstrates that WGS can meet or exceed the performance of these tests and indicates that both current and future precision medicine initiatives could leverage genome data for population-level screening.

Replicating the current SMA testing regime poses a problem for high throughput WGS due to the almost perfect sequence identity between SMN1 and SMN2. Furthermore, frequent gene conversion between SMN1 and SMN2 is thought to lead to hybrid genes. These challenges demand a bioinformatics method that can overcome the difficulties of this region. Two NGS-based tests for SMA carrier detection have been reported. Larson et al. (Validation of a high resolution NGS method for detecting spinal muscular atrophy carriers among phase 3 participants in the 1000 Genomes Project. BMC Med Genet. 2015; 16:100. doi:10.1186/s12881-015-0246-2) used a Bayesian hierarchical model to calculate the probability that the fraction of SMN1-derived reads is equal to or smaller than ⅓ at three base differences between SMN1 and SMN2. The method disclosed in Larson can test for SMA; though since the method does not perform copy number calling, the method is not an ideal solution for carrier screening. Conversely, Feng et al. (The next generation of population-based spinal muscular atrophy carrier screening: comprehensive pan-ethnic SMN1 copy-number and sequence variant analysis by massively parallel sequencing. Genet Med Off J Am Coll Med Genet. 2017; 19(8):936-944. doi:10.1038/gim.2016.215) described a copy number caller for both SMN1 and SMN2 based on targeted sequencing data that closely mimics the current qPCR method. The method of Feng is designed for targeted sequencing and thus requires specialized normalization that limits the method to one assay at one site. The method derives the total copy number of SMN (including both SMN1 and SMN2) from the read coverage in exon 7, and calculates the SMN1:SMN2 ratio based on the numbers of SMN1- and SMN2-supporting reads at the c.840C>T site. Using the total coverage and SMN1:SMN2 ratio, the method derives the absolute copy number of SMN1 and SMN2. Because this method relies solely on a single locus, it is unreliable for WGS data where per-locus depth variability can be very high.

Compared with targeted sequencing, WGS provides a much more uniform coverage across the genome and provides a less-biased approach to detecting copy number variants (CNVs). In addition, WGS offers an opportunity to comprehensively profile the spectrum of population variation in the SMN region, for which the understanding at the sequence level is poor. This example describes a novel method that detects the CN of both SMN1 and SMN2 using WGS data. While most conventional assays only test for the absence of c.840C as a proxy for “exon 7 deletion”, this example describes a method that can more fully characterize the variability in the region including: 1) DNA deletions, including whole gene deletion/duplication and a partial deletion of a region that includes exons 7 and 8; and 2) small variant detection including the g.27134T>G SNP that is correlated with “silent” carriers of SMA (two copies of SMN1 on the same haplotype). The accuracy of this method was demonstrated by comparing CN calls using digital PCR with the WGS-based calls of the example. A concordance of 100% ( 48/48) for SMN1 and 98% ( 47/48) for SMN2 was shown. Additionally, this method was applied to 2,504 unrelated samples from the 1000 Genomes Project and 10,243 unrelated samples from the NIHR BioResource Project to report on the population distributions of SMN1 and SMN2 copy numbers. The carrier frequencies for SMA determined using the method of the example agree with those reported by previous PCR-based studies. In addition to demonstrating the accuracy of the method to quantify variants in the SMN region, the example highlights the importance of using ethnically diverse populations when developing novel informatic methods to resolve difficult clinically relevant regions of the genome.

### Materials and Methods

Samples and Data Processing

Samples validated using digital PCR were procured from the Motor Neuron Diseases Research Laboratory (Nemours Alfred I. duPont Hospital for Children) collection and were generated from cell lines as described previously. This cohort contained 29 SMA samples (14 type I SMA, 1 type I/II SMA, 10 type II SMA, 3 type III SMA and 1 SMA with unknown clinical grade), six non-SMA neuromuscular disease samples (including hereditary sensory and autonomic neuropathy 3, myotonic dystrophy type I, distal hereditary motor neuronopathy type I and Charcot-Marie-Tooth peripheral neuropathy type IA) and 13 normal samples. WGS was performed with TruSeq DNA PCR-free sample preparation with 150 bp paired reads sequenced on Illumina (San Diego, Calif.) HiSeq X instruments. Genome build GRCh37 was used for read alignment.

For population studies, 13,343 individuals were queried from the NIHR BioResource Rare Diseases project (EGAS00001001012), which performed WGS on individuals with rare diseases and their close relatives. Additional individuals (n=840) from the Next Generation Children project (EGAD00001004357), which performed diagnostic trio WGS on patients and their parents from neonatal and paediatric intensive care units in the UK, were also investigated. WGS in these studies was performed using the Illumina TruSeq DNA PCR-Free Sample Preparation kit with 100 bp or 125 bp paired reads sequenced on Illumina HiSeq 2500, or with 150 bp paired reads sequenced on HiSeq X instrument. Genome build GRCh37 was used for read alignment. When doing the population analysis, related individuals and those of unknown ancestry were excluded, leaving 10,243 unrelated individuals.

For the 1000 Genomes Project (1kGP) data, WGS BAMs were downloaded from ncbi.nlm.nih.gov/bioproject/PRJEB31736/. These BAM files were generated by sequencing 2×150 bp reads on Illumina NovaSeq 6000 instruments from PCR-free libraries sequenced to an averaged depth of at least 30× and aligning them to the human reference, hs38DH, using BWA-MEM v0.7.15 (greater than 30× mean genome coverage).

SMN Copy Number Analysis by Orthogonal Methods

For validation samples, SMN1 and SMN2 CNs were measured with the QuantStudio 3D Digital PCR System (Life Technologies, Carlsbad, Calif.) using allele-specific exon 7 probes as described previously. SMN1 and SMN2 copy numbers were normalized against those for RPPH1 (RNase P). Detected SMA samples in the Next Generation Children project were confirmed using standard MLPA (SALSA MLPA P060 SMA Carrier probemix, MRC-Holland).

Copy Number Calling for Intact and Truncated SMN

The SMN1 and SMN2 loci are affected by two common CNVs, the whole gene CNV and a partial gene deletion of exons 7 and 8 (see Results of this example). The truncated form of SMN with the partial deletion of exons 7 and 8 was named SMN*. The method called the copy number of intact SMN1+SMN2 (referred to as SMN hereafter) and truncated SMN (SMN*) genes using the following steps.

Identify and count reads from SMN1 and SMN2: Read counts were calculated directly from the WGS aligned BAM file using all reads mapped to either SMN1 or SMN2, including those with a mapping quality of zero. Frequently reads would align to these regions with a mapping quality of zero because the sequence is identical between the two regions. These two genes only share sequence with each other and not with other regions of the genome. Read counts in a 22.2 kb region encompassing exon 1 to exon 6 were used to calculate the total SMN (SMN1, SMN2 and SMN*) CN, and read counts in the 6 kb region including exon 7 and exon 8 were used to calculate the CN of intact SMN (SMN1 and SMN2).

Calculate normalized depth of the SMN regions: The read counts for the two regions described above were each normalized by region length and further normalized by dividing against the median depth of 3000 pre-selected 2 kb regions across the genome.

Convert normalized depth into copy numbers: Normalized depth values across the population were modeled with a one-dimensional mixture of 11 Gaussians with constrained means that center around each integer copy number value representing copy number states ranging from 0 to 10. Copy numbers of total SMN and intact SMN were called from the Gaussian mixture model (GMM) with a posterior probability threshold of 0.95.

Calculate the CN of the intact and truncated SMN: The intact SMN CN was defined as the CN of the 6.3 kb region covering exons 7 and 8. The copy number of truncated SMN (SMN*) was derived by subtracting the intact SMN CN from total SMN CN calculated from the 22.2 kb region that includes exons 1-6.

Genotyping the Copy Number of Alleles at Single Bases

The number of chromosomes carrying the SMN1 and SMN2 bases was called by combining the total SMN CN with the read counts supporting each of the gene-specific bases. Based on the called copy number of intact SMN at each position, the method iterated through all possible combinations of SMN1 and SMN2 copy number and derives the combination that produces the highest posterior probability for the observed number of SMN1 and SMN2 supporting reads. In addition to calling the CN of bases that are specific to either SMN1 or SMN2, this method can be applied to variant positions to identify the copy number of SNPs known to be specific to one of the two genes, e.g. g27134T>G as described below.

Copy Number of SMN1 and SMN2

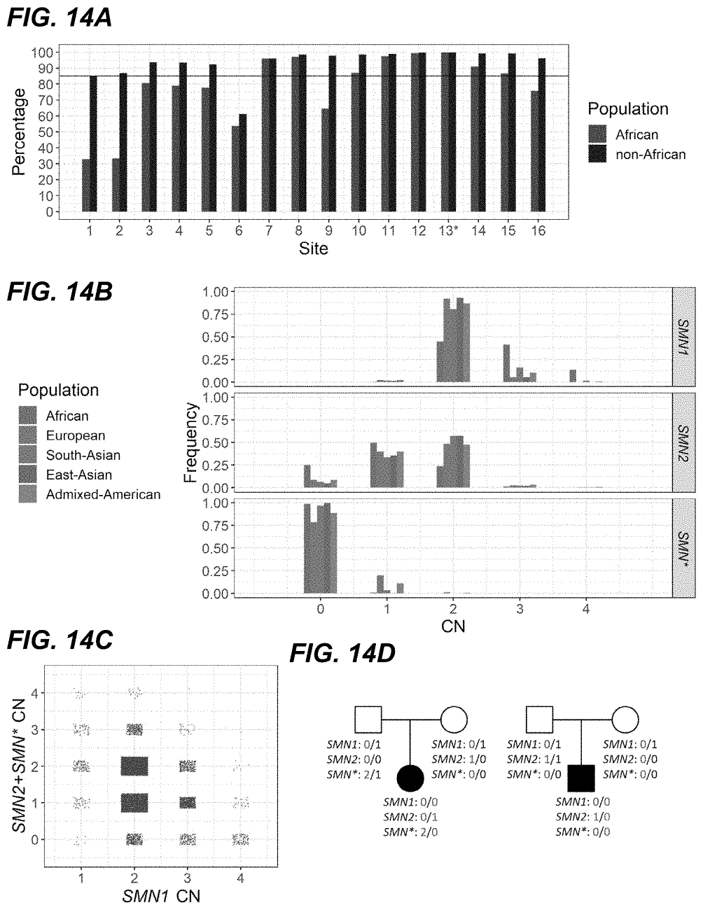

For the 16 positions (localized from intron 6 to exon 8) that are different between SMN1 and SMN2 in the reference genome, whether these sites were truly fixed in the population were tested by comparing the CN call of the SMN1 alleles for these positions with the CN call for the splice variant base SMN1 c.840C. Eight positions, including c.840C>T, where the SMN1 bases are fixed or close to being fixed in the population were identified based on concordance with the splice variant base (see the Results section of this example, FIG. 14A). The remaining sites may be polymorphic in the population and may be unreliable to use for CN calling.

To make a final CN call the method required that either: 1) the SMN1 CN calls agree across at least 5 out of 8 sites at a posterior probability cutoff of 0.8, or 2) at least 5 out of 8 sites (with a posterior probability >0.6) agree with the CN call derived from reads overlapping all 8 sites (with a posterior probability >0.9). Otherwise a no-call was produced for both the SMN1 and SMN2 CNs. SMA samples were identified as having zero copy of intact SMN1, and carrier samples were identified as having one copy of intact SMN1.

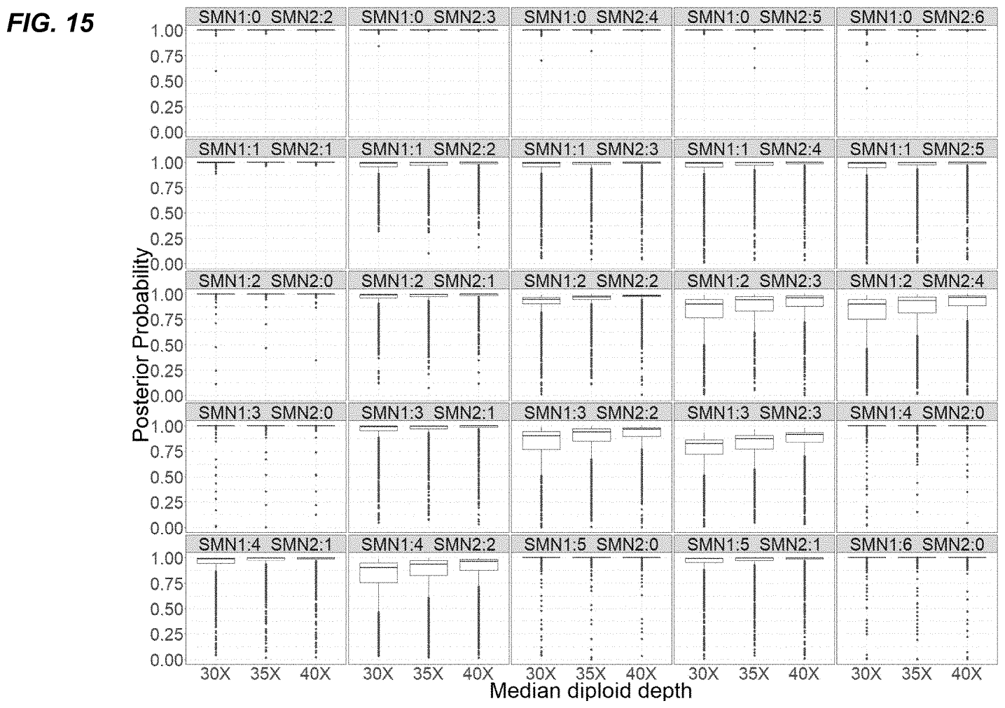

At higher CN values, greater variability in read depth would be expected, leading to less confident CN calls (with a lower posterior probability) at individual sites and more disagreement between sites. As a result, no-calls were more likely to be made in samples with high SMN1/SMN2 CNs, i.e. both values larger than or equal to two (see FIG. 15). However, in such samples determining confidently whether the SMN1 CN was or was not 0 (SMA) or 1 (carrier) was still possible, which allowed calling SMA/not SMA or carrier/not carrier. When the SMN1 copy number was a no-call, if at least seven of the SMN1 CN calls were confidently greater than zero then the sample was called “not SMA”. Similarly, if at least seven of the SMN1 CNs were confidently greater than one, the sample was called “not carrier”. Additionally, when the SMN1 CN was a no-call, the absence of the c.840C allele that would be indicative of SMA was directly tested. This was done by testing whether the number of reads supporting the SMN1 base (c.840C) was more likely to derive from zero or one copy of SMN1.

### Results

Common CNVs Affecting the SMN1/SMN2 Loci

The genes SMN1 and SMN2 reside in an 2 Mb region in the reference genome with a large number of complicated segmental and inverted segmental duplications. While existing methods (e.g., PCR-based methods) focus primarily on the c.840C>T site, this example illustrates a copy number approach based on the sequencing data from the full genes. The number of SMN1 copies was defined as the number of SMN genes that carry the c.840C allele, and the number of SMN2 copies was defined as the number of SMN genes with c.840T allele. The sequence analysis was performed using the high depth (>30×) WGS data from 2504 samples from the 1000 Genomes Project (1kGP), as well as the 10,243 unrelated samples from the NIHR BioResource Project (see the Methods of this example).

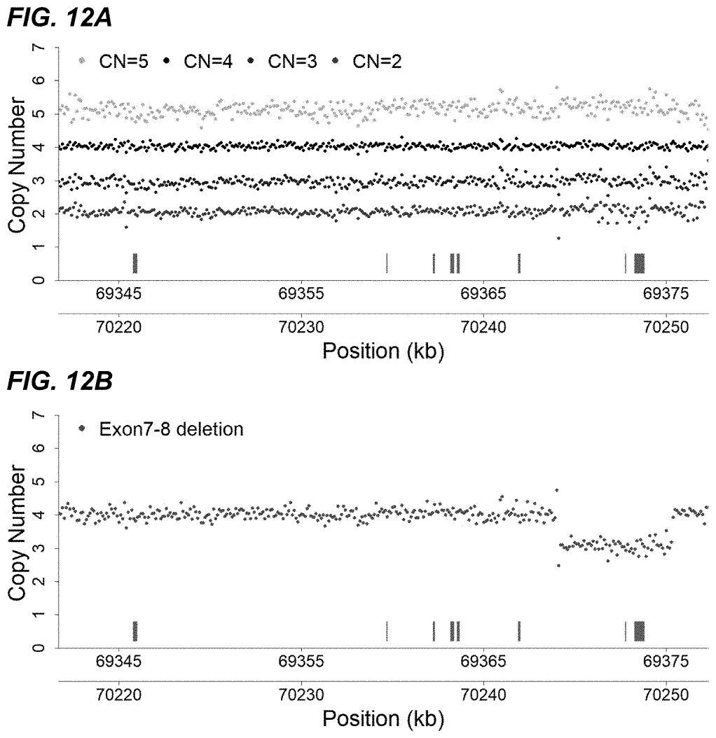

To formulate the CN calling strategy, two common CNVs that lead to DNA deletions were first characterized. The primary CNV assessed involves the entire SMN1/SMN2 gene region. The read depth across the 30 kb homologous region harboring SMN1 and SMN2 genes were examined. FIG. 12A shows the normalized read depths, in 100 bp sliding windows, in samples with different SMN1+SMN2 CNs across this region (representing both SMN1 and SMN2). The depth profile shows that this entire region was deleted or duplicated in these samples. The exact breakpoints of this CNV were expected to vary from sample to sample due to the extensive sequence homology within and beyond this region and can only be resolved in high resolution with long read sequencing. For SMA testing, the analysis was restricted to the (˜30 kb) regions that include the SMN genes (SMN1 or SMN2).

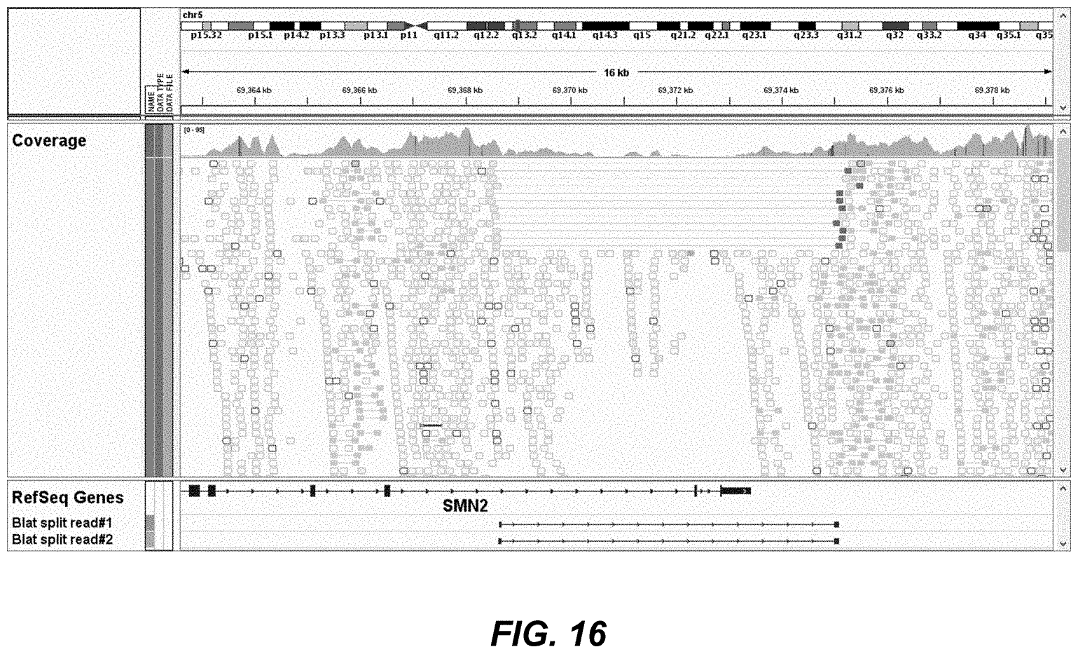

In addition to whole gene CNVs, a 6.3 kb partial gene deletion encompassing both exons 7 and 8 (FIG. 12B, FIG. 16) was found. The sequences at the breakpoint are identical between SMN1 and SMN2, so this deletion occurs at either chr5: 70244114-70250420 in SMN1 or chr5: 69368689-69375000 in SMN2 (FIG. 16, hg19). However, about 500 bp downstream from the breakpoint that defines the end of this deletion there are three base differences between the SMN1 and SMN2 loci (70250881A>69375425C, 70250981A>69375525G, 70250991A>69375535G). Among the samples that contain this deletion, 245 read pairs from 237 samples where one read spanned the breakpoint and the other spanned at least two of the three SMN-differentiating bases were identified. Analysis of these read pairs revealed that 100% were consistent with the deletion occurring on the SMN2 sequence background. This truncated form of SMN2 was named “SMN*” and since both exons 7 and 8 are deleted, SMN* most likely has limited or no biological function. Therefore, SMN* is an important variant that any SMN CN caller should take into account.

FIGS. 12A and 12B show non-limiting exemplary plots illustrating common CNVs affecting the SMN1/SMN2 loci. FIG. 12A shows depth profiles across the SMN1/SMN2 regions. Samples with a total SMN1+SMN2 copy number of 2, 3, 4 and 5 are shown as dots, respectively. Depth from 50 samples are summed up for each CN category. Each dot represents normalized depth values in a 100 bp window. Read counts are calculated in each 100 bp window, summing up reads from both SMN1 and SMN2, and normalized to the depth of wild-type samples (CN=4). The SMN exons are represented as purple boxes. The two x axes show coordinates in SMN1 (bottom) and SMN2 (upper). FIG. 12B shows depth profiles aggregated from 50 samples carrying a deletion of exons 7 and 8 are shown as dots. Read depths are calculated in the same way as in FIG. 12A.

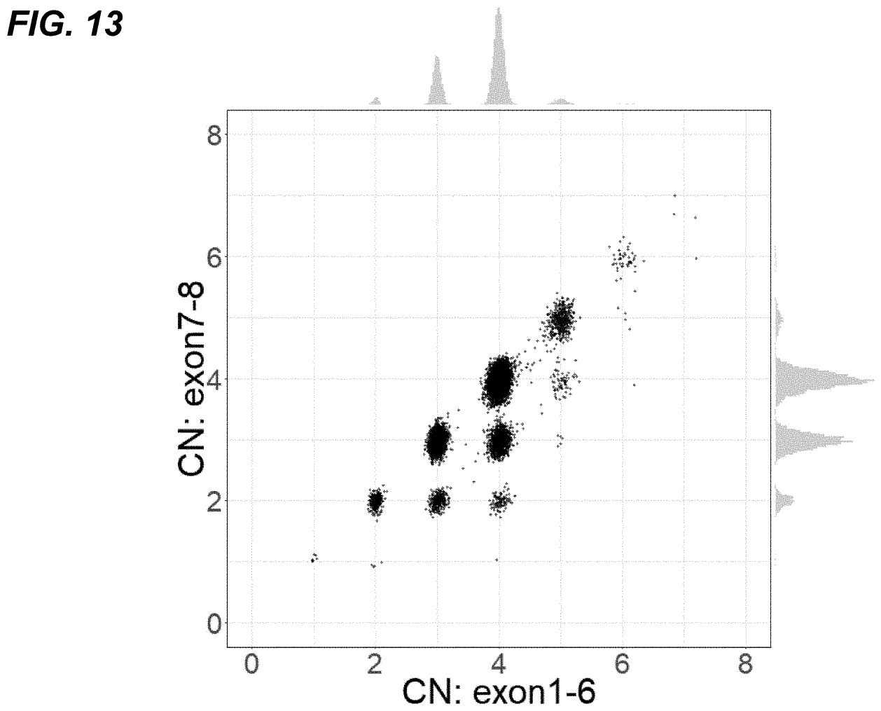

After searching for anomalous read pairs, no other common CNVs in the SMN region was found. Combining this information together, CNs of the SMN genes were called to specifically identify the number of intact and truncated forms by dividing the genes into two regions: the 6.3 kb region that includes exons 7-8 and the 22.2 kb region that includes exons 1-6. The CNs of these two regions were calculated from read depth as described in the Methods section of this example. The CN calculated from the exons 7-8 region provided the number of intact SMN genes. Samples with SMN* had a higher CN call from the exon 1-6 region compared to the CN call from the exon 7-8 region, and their difference represented the CN of SMN*. FIG. 13 shows the results of this calculation for 12,747 sample cohort where 2,144 instances of SMN*, including 140 samples with two copies of SMN* and one sample with three copies of SMN*, were identified.

FIG. 13 shows a non-limiting exemplary scatterplot of total SMN (SMN1+SMN2) copy number (x axis, called by read depth in Exon 1-6) and intact SMN copy number (y axis, called by read depth in Exon 7-8).

Differentiating SMN1 from SMN2 CNs

After calculating the total number of copies of SMN genes, SMN1 and SMN2 were differentiated as described below. Since c.840C>T is the most important functional difference between SMN1 and SMN2, the absolute copy number of these two genes can theoretically be derived using the ratio between the number of reads supporting SMN1 and SMN2 at this site. However, the read depth at one diploid position is typically 30-40× for a WGS dataset and sometimes does not provide sufficient power to clearly differentiate between different CN states (see FIG. 15). Therefore, additional base differences near c.840C>T were utilized so that information at these sites can be combined with c.840C>T when making a CN call. Because differentiating between intact SMN1 from SMN2 was desired, variants that occur within the 6.3 kb deletion were considered. SNPs in homopolymers and short tandem repeats (STRs) that may be more prone to errors were excluded, resulting in 16 base differences between SMN1 and SMN2 (Table 8).

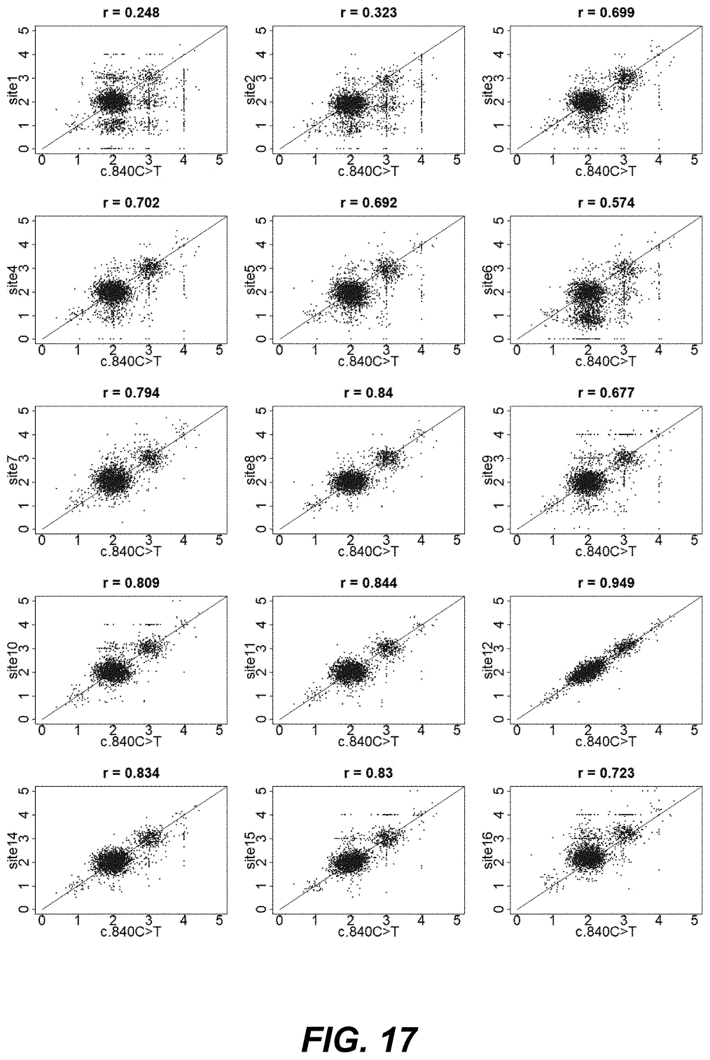

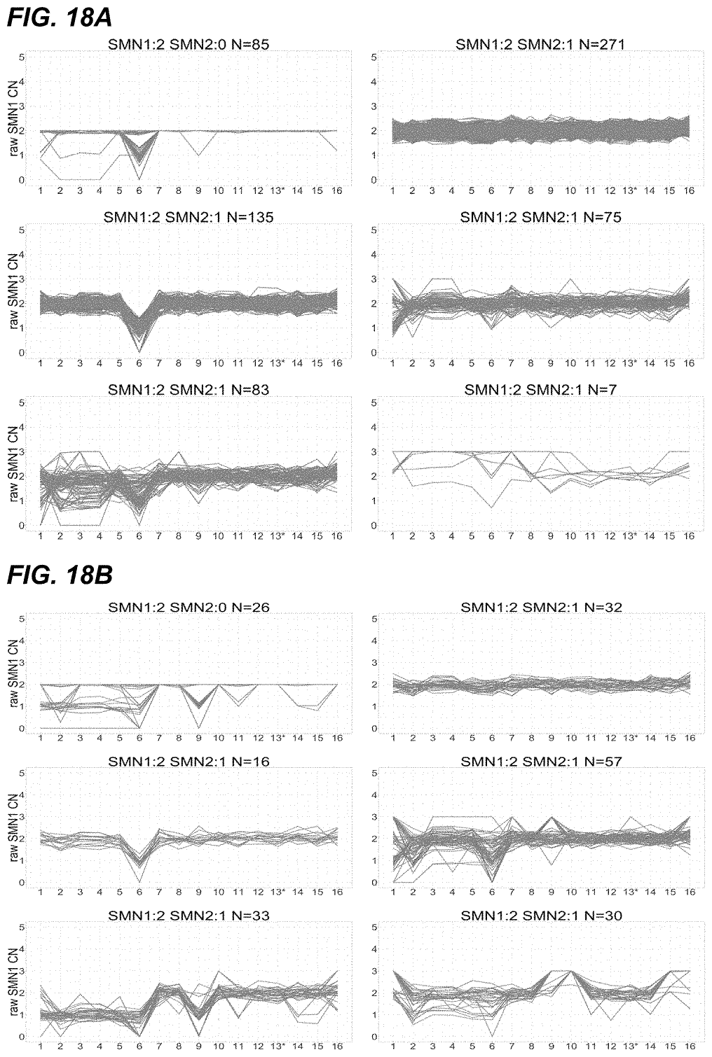

For these 16 base differences, the CNs of the SMN1 and SMN2 alleles were independently call (see the Methods section of this exampled), and the CN calls for each position with the CN calls at the splice variant site were compared (FIG. 14A, FIG. 17). There was a notable difference between the concordance of calls in the African and non-African populations (FIG. 14A). For the non-African samples, there were 13 sites that had high (>85%) CN concordance with the splice site. Conversely, for the African samples there were only seven sites that had high CN concordance with the splice site, and the concordance values were lower than in non-African populations. This is consistent with within-gene variation at many of these positions and higher frequencies for these non-reference alleles in the African population. The splice variant and the seven positions that were highly concordant with the splice variant in both African and non-African populations were selected to make CN calls on SMN1 and SMN2. By restricting to two CN states that allow easy identification of hybrid alleles (SMN1=CN2 and SMN2=CN or SMN1=CN2 and SMN2=CN1), estimating the allele frequencies of these sites on the SMN1 and SMN2 genes (Table 9, FIGS. 18A and 18B) was possible. Based on this analysis, across these eight positions, up to 0.5% of the SMN1 genes were estimated contain an SMN2 allele. Conversely, up to 0.9% of the SMN2 genes were estimated to carry an SMN1 allele. These observations may be the result of gene conversion or many of these eight sites being polymorphic in the population. A large portion of these hybrid alleles come from African populations (Table 9).

FIGS. 14A-14D illustrate the distributions of SMN/SMN2/SMN* copy numbers in the population. FIG. 14A is a non-limiting exemplary plot illustrating the percentage of samples showing CN call agreement with c.840C>T across 16 SMN1-SMN2 base difference sites in African and non-African populations. Site 13* is the c.840C>T splice variant site. The black horizontal line denotes 85% concordance. FIG. 14B shows non-limiting exemplary histograms of the distributions of SMN1, SMN2 and SMN* copy numbers across five populations in 1kGP and the NIHR BioResource cohort (numbers shown in Table 15). FIG. 14C is a non-limiting exemplary plot of SMN1 CN vs. total SMN2 CN (intact SMN2+SMN*). FIG. 14D shows two trios with an SMA proband detected by the caller and orthogonally confirmed in the NIHR BioResource cohort. CNs per allele of SMN1, SMN2 and SMN* are phased and labeled for each member of the trios.

Introduction of more base differences improved the ability to differentiate SMN1 from SMN2. However because these sites are not truly invariant in the respective genes and CN calling at single sites can be subject to error, the likelihood that one of the individual calls would deviate from the true copy number state would be increased. To make a final call, the SMN1 CN calls were required to agree with each other at 5 or more of 8 sites (see the Methods section of this example for a full description of the rules for CN calling). With a posterior probability cutoff of 0.8, the majority of samples had consistent calls at least five out of the eight sites and only 1.4% of samples had fewer than 5 sites that agreed (Table 10). In 80% of those samples, a confident CN call was made based on the second consensus rule (requiring agreement with the CN call made by summing up reads at all 8 sites). The “non-agreeing” sites were more frequently no-calls due to a low posterior probability rather than discrepant calls, and only 15.3% of them were confident calls that disagreed with the consensus of the other sites. Again, a large portion of the disagreements come from African populations (Table 10). Using fewer sites for the majority rule produced a larger number of no-calls and wrong calls compared with using eight sites (Table 11).

Validation of the SMN Copy Number Caller

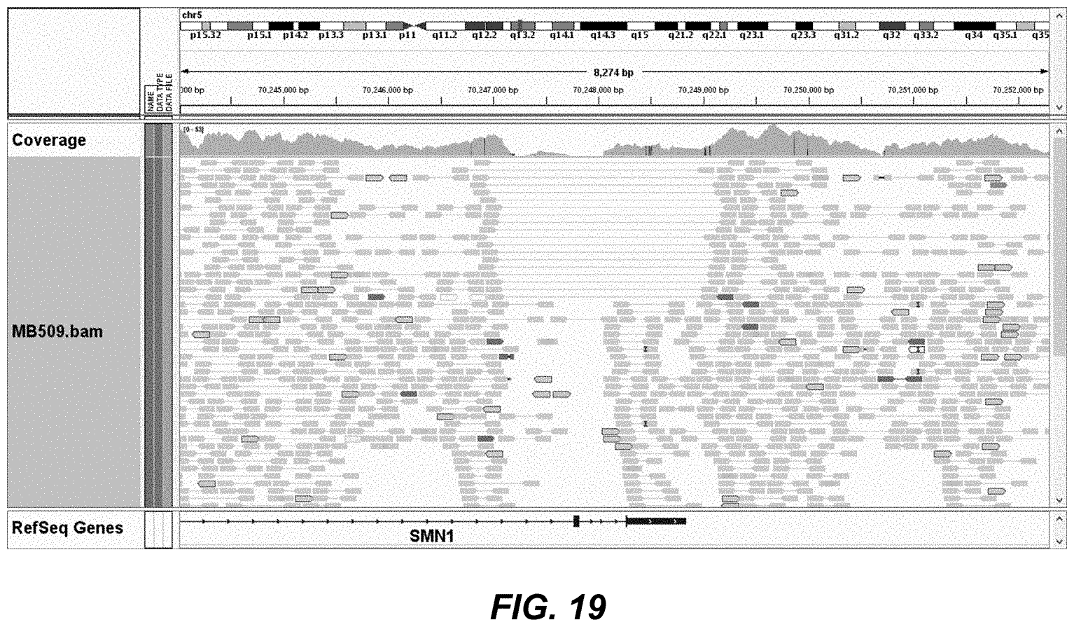

To test the method, 48 samples with known SMN1 and SMN2 CNs, including 29 SMA probands, 6 SMA carriers and 13 samples with an SMN1 CN larger than 1, were sequenced. The SMN1 CN calls agree with digital PCR results in all of the 48 cases, and the SMN2 CN calls agree in 47 (97.9%) of the 48 cases (Tables 6A and 6B). In this single discrepant case (MB509), the method called an SMN2 CN of 3 while digital PCR showed an SMN2 CN of 2 (Table 12). Upon closer examination, a 1884 bp deletion in SMN1 (chr5:70247145-70249029, hg19) in this sample (FIG. 19) was found. The deletion is small (does not change the depth significantly in the 6 kb region used for determining the intact SMN CN) and has not been previously reported (nor found in the population data), so the method was not designed to detect it. As a result, this sample was identified correctly as SMA but the SMN2 CN was overestimated by one. The deletion is consistent with the CN calls made in the 8 SMN1-SMN2 difference sites, where the first 2 sites are not in the deletion and called at SMN1 CN=1 and the next 6 sites are in the deletion and called at SMN1 CN=O.

The consistency of SMN1/SMN2/SMN* CN calls in 258 trios from the Next Generation Children project cohort were analyzed (see the Methods section of this example). There was no Mendelian error in any of the calls (Table 13).

TABLE 6A
Validation against samples with known SMN1/SMN2 CNs.

| | CN by digital | | | | |
|---|---|---|---|---|---|
| | PCR | Total | Concordant | Discordant | Agreement |
| SMN1 | 0 | 29 | 29 | 0 | 100.0% |
| | 1 | 6 | 6 | 0 | 100.0% |
| | 2 | 10 | 10 | 0 | 100.0% |
| | 3 | 3 | 3 | 0 | 100.0% |
| | Total | 48 | 48 | 0 | 100.0% |
| SMN2 | 0 | 1 | 1 | 0 | 100.0% |
| | 1 | 4 | 4 | 0 | 100.0% |
| | 2 | 29 | 28 | 1 | 96.6% |
| | 3 | 11 | 11 | 0 | 100.0% |
| | 4 | 3 | 3 | 0 | 100.0% |
| | Total | 48 | 47 | 1 | 97.9% |

TABLE 6B
Validation against samples with known
SMN1/SMN2 copy numbers (CNs).

| | CN by | | | | |
|---|---|---|---|---|---|
| | orthogonal | | Concor- | Discor- | |
| | method | Total | dant | dant | Agreement |
| SMN1 | 0 | 64 | 64 | 0 | 100.0% |
| | 1 | 45 | 44 | 1 | 97.8% |
| | 2 | 897 | 897 | 0 | 100.0% |
| | 3 | 174 | 174 | 0 | 100.0% |
| | 4 | 43 | 43 | 0 | 100.0% |
| | 6 | 1 | 0 | 1 | 0.0% |
| | Total | 1224 | 1222 | 2 | 99.8% |
| SMN2 | 0 | 117 | 117 | 0 | 100.0% |
| | 1 | 466 | 465 | 1 | 99.8% |
| | 2 | 541 | 539 | 2 | 99.6% |
| | 3 | 60 | 60 | 0 | 100.0% |
| | 4 | 9 | 8 | 1 | 88.9% |
| | Total | 1193 | 1189 | 4 | 99.7% |
| SMN2Δ7-8 | 0 | 1089 | 1089 | 0 | 100.0% |
| | 1 | 80 | 80 | 0 | 100.0% |
| | 2 | 4 | 4 | 0 | 100.0% |
| | Total | 1173 | 1173 | 0 | 100.0% |

Copy Number of SMN1, SMN2 and SMN* by Population

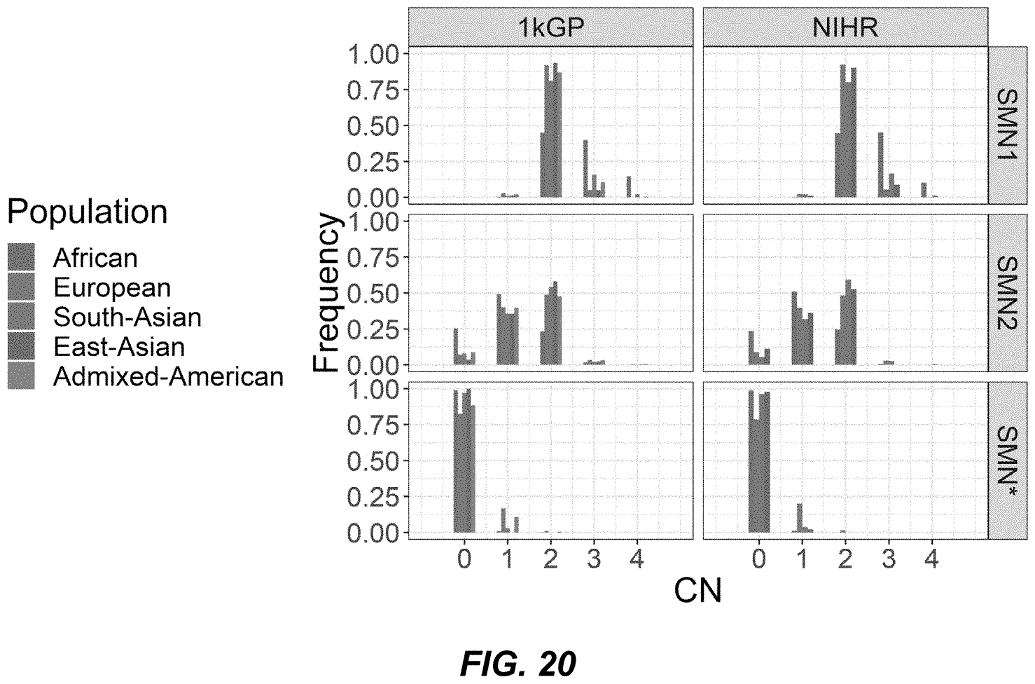

Given the high accuracy demonstrated by the validation against digital PCR results, the method was applied to high depth (>30×) WGS data from 12,747 unrelated samples from the 1kGP and the NIHR BioResource Project (Table 14). The CN distributions were analyzed by population (Europeans, Africans, East Asians, South Asians and admixed Americans consisting of Colombians, Mexican-Americans, Peruvians and Puerto Ricans). FIG. 14B shows the histogram of the number of individuals with different CNs of intact SMN1, intact SMN2 and SMN*. The distributions are similar between the 1kGP samples and the NIHR BioResource samples (FIG. 20). In general, individuals had more copies of SMN than SMN2. The most common combinations of SMN1/SMN2 copy number were 2/2 (44.9%) and 2/1 (33.4%). Excluding the Africans that showed higher variability in both SMN1 and SMN2 CN, the variability of SMN1 copy number was much lower than that of SMN2 copy number. Conversely, 54.7% of Africans had three or more copies of SMN1, which was more than double what was observed in any of the other four populations (FIG. 14B, Table 7). There was an inverse relationship between the copy number of SMN1 and SMN2, where the CN of SMN2 was lower with increasing CN for SMN1 (FIG. 14C, correlation coefficient −0.344, p-value <2.2e-16). This observation is consistent with a mechanism where gene conversion occurs between SMN1 and SMN2. The observed higher SMN1 CN relative to SMN2 CN could be a result of a bias towards SMN2-to-SMN1 conversion or selection against a low SMN1 CN. Africans have significantly lower SMN2 CN than the other populations.

The number of SMA carriers identified across populations is summarized in Table 7 and Table 15. In 12,683 individuals with confident SMN1/SMN2 CN calls, Europeans had the highest carrier frequency at 2.2%, followed by admixed Americans (2.05%), East Asians (1.35%) and South Asians (1.67%). Africans had the lowest carrier frequency (0.44%). The CN frequency distributions observed in this example are consistent with previous studies of SMN1/SMN2 CN distributions in the general population. In addition, the frequency of the exon 7-8 deletion (SMN*) across populations was determined: 21.2% of Europeans and 11.5% of admixed Americans had at least one copy of SMN*, while the frequency was lower in South Asians (3.35%), Africans (1.1%) and East Asians (0.34%).

In the Next Generation Children project cohort (see the Methods section of this example), SMA in two neonatal probands from trio analysis, which were confirmed independently, were identified. In addition, SMN1, SMN2 and SMN* CNs were phased for each trio member (FIG. 14D).

Simulation for Single Site CN Calling

The numbers of reads at one single site at a sample median depth of 30×, 35× and 40× were simulated based on a Poisson distribution, and SMN1 supporting reads were sampled based on a binomial model with all possible combinations of SMN1 CN and SMN2 CN with the total SMN CN being between 2 and 6. With the numbers of supporting reads for SMN1 and SMN2, the posterior probability of the simulated SMN1 CN was derived (see the Methods section of this example). The posterior probability was high (greater than 0.9) when at least one value of SMN1 or SMN2 CN was low (smaller or equal to 1) (FIG. 16). When both values were larger than 2, i.e. in SMN1:SMN2 combinations of 2:2, 2:3, 2:4, 3:2, 3:3, and 4:2, the posterior probability frequently became low and fell below 0.9. This resulted from the higher variability in read depth when the expected CN is higher. Therefore, in these scenarios making SMN1 and SMN2 CN calls using one single site may be less accurate.

Discrepancies in Validation Samples

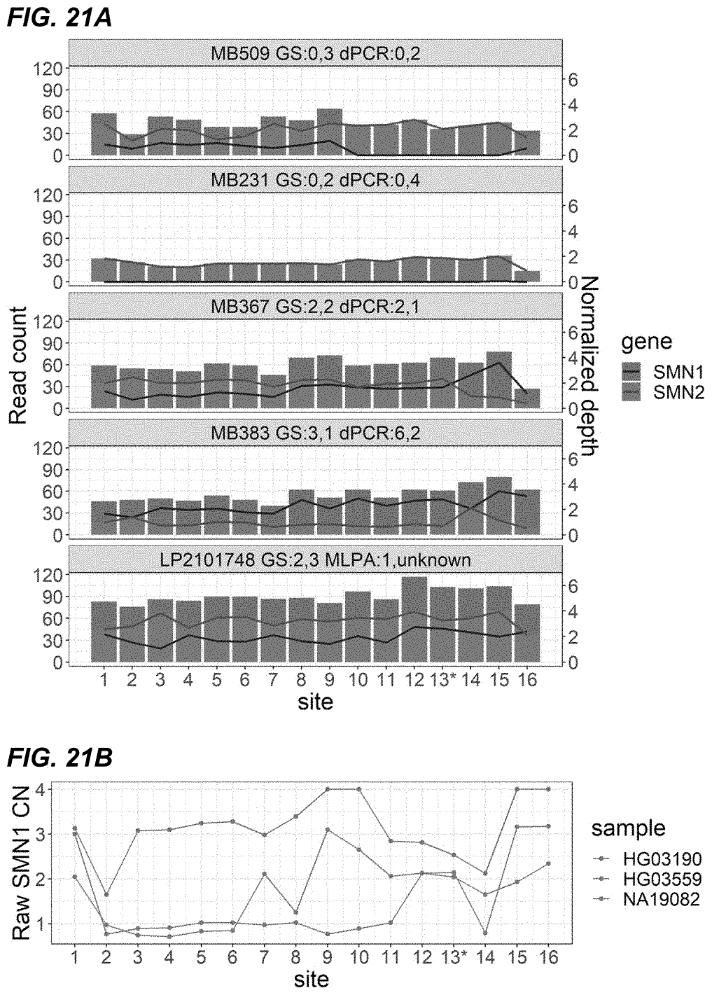

There was one sample, MB509, that was discrepant between our CN call and the digital PCR results. Upon further inspection, this sample was found to have two copies of SMN2 and one copy of SMN1 with a 1884 bp deletion (chr5:70247145-70249029, hg19, FIG. 20). While read alignments in the SMN1/2 region is not always accurate, careful analysis of the split reads showed that the reads or their mates overlapped bases that were specific to SMN1. Without intending to be limited by the theory, it was hypothesized that this deletion was correctly placed on SMN. The deletion is small (does not change the depth significantly in the 6.3 kb region used for determining the intact SMN CN) and has not been previously reported (nor found in the 1kGP samples, thus a very rare variant), so the method was not designed to detect the deletion. As a result, the method called the total copy number of SMN1+SMN2 as 3. The deletion is consistent with the CN calls made in the 8 SMN1-SMN2 difference sites, where the first 2 sites were not in the deletion and called at SMN1 CN=1 and the next 6 sites were in the deletion and called at SMN1 CN=0 (FIG. 21A). Based on the majority rule, the method called the SMN1 copy number as 0, correctly identifying the sample as SMA. The SMN2 copy number was calculated as the total copy number minus the SMN1 copy number, so the method called the SMN2 copy number to be 3, overestimating it by 1.

Four other samples, MB231, MB367, MB383 and LP2101748, had discrepancies between the CN calls made and results from either digital PCR or MLPA. Read counts and normalized depth values (read counts divided by haploid sample depth) at the 8 base difference sites supported our CN calls (FIG. 21A) and the discrepancy was likely to be caused by errors in the orthogonal methods. In two samples, genome sequencing (GS) calls and digital PCR calls differed by a factor of two (MB231: GS-0,2, PCR-0,4 and MB383: GS-3,1, PCR-6,2). There may be a normalization issue with digital PCR, leading to an overestimation of copy number by two-fold.

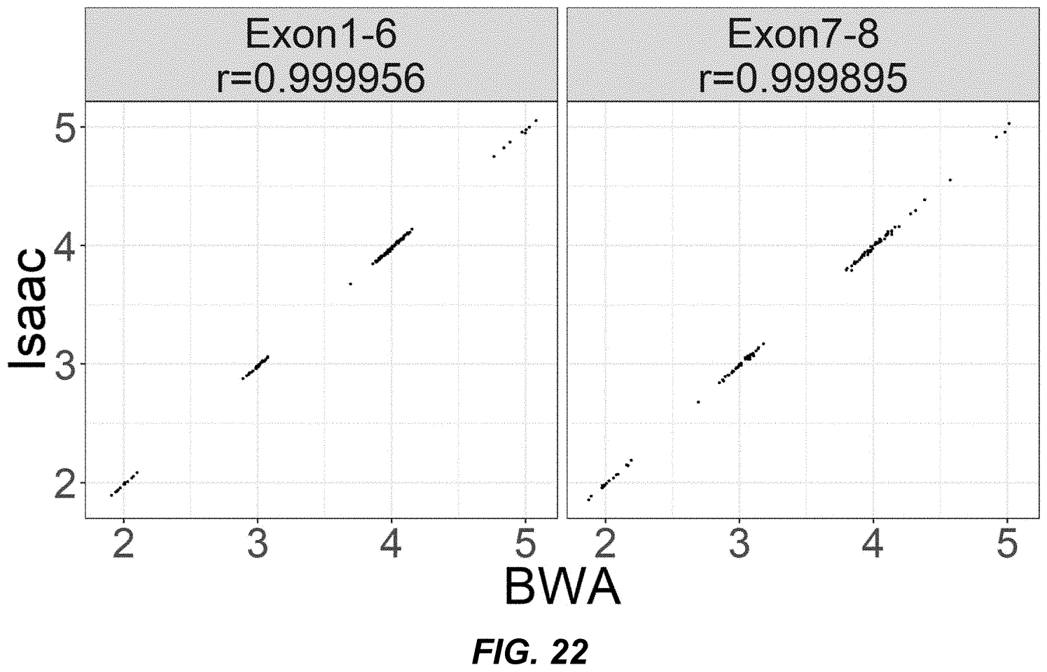

When comparing the CN calls made with MLPA results in 1109 1kGP samples, one sample was excluded where a no-call for SMN2Δ7-8 was made due to low posterior probability of the total SMN CN, as well as three samples where a no-call for SMN1 and SMN2 CN was made due to disagreements in the CN calls across the 8 selected sites that failed to meet the consensus rules (FIG. 22B).

Detection of “Silent” Carriers

The g.27134T>G SNP may be associated with the 2+0 SMA silent carrier status where one chromosome carries two copies of SMN1 (either by SMN1 duplication or gene conversion of SMN2 to SMN1) and the other chromosome has no copies of SMN. The method of this example can also detect the presence of this SNP and thus can be used to screen for potential silent carriers. This SNP was most strongly associated with two-copy SMN1 alleles in Africans, where 84.5% of individuals with three copies of SMN1 and 92.6% of individuals with four copies of SMN1 have the g.27134T>G SNP (Table 7). Calling this SNP greatly increased the carrier detection rate in Africans as Africans have a higher frequency of alleles carrying two copies of SMN1 (Table 17 and Table 18). However, 33% of individuals with two copies of SMN1 also had the g.27134T>G SNP, suggesting that a significant portion of singleton SMN1 alleles also carry this SNP. Maximum likelihood estimates for the percentages of singleton and two-copy SMN1 alleles that carry g.27134T>G (Table 17) and residual risks for the combination of CN and SNP calling (Table 18) were calculated. The calculated estimates are similar to previous studies, though there is considerable variability across all of these estimates. This variability may be driven by population variability, e.g. Africans (this example) vs. African Americans (previous studies), and Northern Europeans (overrepresented in this example) vs. more diversely sampled Caucasians (previous studies).

TABLE 7
SMN1 CN and g.27134T > G frequency by population.

| | | SMN1 CN = 1 | | SMN1 CN = 2 | | | SMN1 CN = 3 | | | SMN1 CN = 4 | | |
|---|---|---|---|---|---|---|---|---|---|---|---|---|
| Ethnicity | Total | Count | g.27134T > G+ | Count | g.27134T > G+ | | Count | g.27134T > G+ | | Count | g.27134T > G+ | |
| African | 902 | 4 | 0(0.0%) | 404 | 134 | (33.17%) | 373 | 315 | (84.45%) | 121 | 112 | (92.56%) |
| European | 9648 | 212 | 0(0.0%) | 8899 | 4 | (0.04%) | 524 | 22 | (4.2%) | 13 | 2 | (15.38%) |
| South-Asian | 1199 | 20 | 0(0.0%) | 965 | 1 | (0.1%) | 195 | 5 | (2.56%) | 19 | 1 | (5.26%) |
| East-Asian | 593 | 8 | 0(0.0%) | 552 | 1 | (0.18%) | 33 | 1 | (3.03%) | 0 | 0 | (NA) |
| Admixed-American | 341 | 7 | 0(0.0%) | 296 | 7 | (2.36%) | 36 | 9 | (25.0%) | 2 | 1 | (50.0%) |

Comparison Between Two Aligners, BWA and Isaac

The method of this example analyzed reads permissively in both SMN1 and SMN2, and thus was insensitive to how the aligner differentiates between the two genes. Therefore, using different aligners should produce similar results. The BAM data analyzed in this example were generated using two different aligners: BWA for the 1kGP data and various versions of Isaac for the rest. The consistent SMN1/2 CN distributions between 1kGP and NIHR (Table 19, FIG. 20) samples suggests that our method is insensitive to the aligner. Additionally, the method was tested for consistency by aligning 117 samples with both BWA and Isaac, including 5 SMA samples and 3 carriers. All 117 samples produced the exact same calls (SMN1/SMN2/SMN2Δ7-8 CN) with the method of this example and the normalized depths for both Exon1-6 and Exon7-8 were virtually identical (Pearson's r>0.999, FIG. 22). Comparison between carrier calls by this study and Larson et al.

The carrier calls made in the 1kGP samples in this example (N=37) were compared to those reported by Larson et al. (N=36), and found 26 overlapping calls (Table 15). Presuming the calls made by the method of this example are correct, Larson et al. made 10 false positives (FP) and 11 false negatives (FN) calls. Larson et al. identified carriers by determining whether the fraction of SMN1 supporting reads was smaller than or equal to ⅓. That study used low depth sequencing data which would be expected to result in some errors but, more importantly, their approach is prone to error without calling the total copy number. For example, a sample with one copy of SMN1 and one copy of SMN2 would be called as a non-carrier (SMN1 fraction ½), and a sample with two copies of SMN1 and four copies of SMN2 will be called as a carrier (SMN1 fraction ⅓), resulting in false positive and false negatives (Table 16).

### Additional Figures and Tables

FIG. 15 shows non-limiting exemplary plots each illustrating a distribution of posterior probability for simulated SMN1 CN using a single site at different read depths and SMN1:SMN2 CN combinations

FIG. 16 shows a non-limiting exemplary IGV snapshot of the SMN2 region in a sample with the exon 7-8 deletion. Horizontal lines join two reads in a pair in the center alignment track. BLAT results of two split reads spanning the breakpoint are shown in the bottom track, showing two segments of the same read aligning to either side of the deletion breakpoint.

FIG. 17 shows non-limiting exemplary plots illustrating correlation between raw SMN1 CNs at 15 base differences near c840.C>T and raw SMN1 CNs at the c840.C>T site. The raw SMN1 CN at each site was calculated as the CN of intact SMN times the fraction of SMN1 supporting read counts out of SMN1+SMN2 supporting read counts. Correlation coefficients are listed in the title of each plot.

FIGS. 18 and 18B show non-limiting exemplary plots illustrating SMN1/SMN2 haplotypes in samples with SMN1:2 SMN2:0 and SMN1:2 SMN2:1 in 1kGP. The y axis shows the raw SMN1 CNs as defined in FIG. 16. The x axis shows the 16 sites whose indices are listed and explained in Table 8. Index #13 represents the c840.C>T site. Samples with SMN1:2 SMN2:0 are shown together in the upper left plot. Samples with SMN1:2 SMN2:1 are shown as 5 clusters. FIG. 18A. Non-Africans. FIG. 18B. Africans.

FIG. 19 shows a non-limiting exemplary IGV snapshot showing a 1.9 kb deletion in SMN1 in MB509.

FIG. 20 shows a non-limiting exemplary plot illustrating SMN1/SMN2/SMN* CNs in 1kGP and NIHR cohorts.

FIGS. 21A and 21B show discrepancies and no-calls in validation samples. FIG. 21A show five samples with discrepancies between GS calls and digital PCR or MLPA results. The x axis shows the 16 sites whose indices are listed and explained in Table 8. Index #13 represents the c840.C>T site. The left y axis for the bars shows the supporting read counts for SMN1 and SMN2. The right y axis for the lines shows the normalized read depth, a proxy for copy number, for SMN1 and SMN2 (read counts divided by haploid depth). The title of each panel shows the GS and digital PCR/MLPA calls for each sample, for SMN and SMN2, separated by a comma. FIG. 21B shows three 1kGP validation samples where the SMN caller made no-calls on SMN1 and SMN2 CN due to disagreements among SMN1/SMN2 base difference sites. The eight sites used for the consensus rules of the method are #7-8 and #10-15. The y axis shows the raw SMN1 CNs as defined in FIG. 17.

FIG. 22 shows CN calls derived from BWA and Isaac BAMs.

TABLE 8
Genome coordinates of base differences
between SMN1 and SMN2.

| | | | SMN1 | | SMN2 | |
|---|---|---|---|---|---|---|
| | | | Position, | | Position, | |
| Index | Location | Selected | hg19 | Base | hg19 | Base |
| 1 | Intron 6 | | 70244142 | A | 69368717 | G |
| 2 | Intron 6 | | 70245876 | T | 69370451 | C |
| 3 | Intron 6 | | 70246016 | G | 69370591 | A |
| 4 | Intron 6 | | 70246019 | T | 69370594 | C |
| 5 | Intron 6 | | 70246156 | G | 69370731 | A |
| 6 | Intron 6 | | 70246167 | T | 69370742 | C |
| 7 | Intron 6 | yes | 70246320 | G | 69370895 | A |
| 8 | Intron 6 | yes | 70246793 | G | 69371368 | A |
| 9 | Intron 6 | | 70246919 | A | 69371499 | C |
| 10 | Intron 6 | yes | 70247219 | G | 69371799 | A |
| 11 | Intron 6 | yes | 70247290 | T | 69371870 | C |
| 12 | Intron 6 | yes | 70247724 | G | 69372304 | A |
| 13 | Exon 7 | yes | 70247773 | C | 69372353 | T |
| | (c.840C > T) | | | | | |
| 14 | Intron 7 | yes | 70247921 | A | 69372501 | G |
| 15 | Intron 7 | yes | 70248036 | A | 69372616 | G |
| 16 | Exon 8 | | 70248501 | G | 69373081 | A |

TABLE 9
Frequencies of SMN1 haplotypes with SMN2 allele and SMN2
haplotypes with SMN1 allele in two simple CN states (SMN1 =
CN2 and SMN2 = CN0 or SMN1 = CN2 and SMN2 = CN1).
Numbers in parentheses indicate those contributed by African populations.

| | # SMN1 | # SMN1 | | | # SMN2 | # SMN2 | | |
|---|---|---|---|---|---|---|---|---|
| | haplotypes | haplotypes | | | haplotypes | haplotypes | | |
| | with | with | | Per- | with | with | | Per- |
| Site | confident | SMN2 | | cent- | confident | SMN1 | | cent- |
| index | CN call | allele | | age | CN call | allele | | age |
| 1 | 12292 | 490 | (71) | 4 | 5041 | 101 | (34) | 2 |
| 2 | 9372 | 542 | (79) | 5.8 | 3669 | 46 | (0) | 1.3 |
| 3 | 11784 | 187 | (48) | 1.6 | 4788 | 48 | (1) | 1 |
| 4 | 11056 | 205 | (51) | 1.9 | 4428 | 43 | (1) | 1 |
| 5 | 10212 | 312 | (51) | 3.1 | 4087 | 34 | (1) | 0.8 |
| 6 | 9974 | 1787 | (111) | 17.9 | 3946 | 28 | (1) | 0.7 |
| 7 | 11956 | 58 | (0) | 0.5 | 4874 | 45 | (3) | 0.9 |
| 8 | 12218 | 15 | (1) | 0.1 | 5005 | 8 | (0) | 0.2 |
| 9 | 11872 | 79 | (47) | 0.7 | 4831 | 56 | (35) | 1.2 |
| 10 | 12484 | 2 | (0) | 0 | 5137 | 39 | (29) | 0.8 |
| 11 | 11964 | 19 | (5) | 0.2 | 4880 | 1 | (0) | 0 |
| 12 | 12506 | 1 | (1) | 0 | 5148 | 0 | (0) | 0 |
| 13 | 12836 | 0 | (0) | 0 | 5313 | 0 | (0) | 0 |
| 14 | 12386 | 9 | (6) | 0.1 | 5088 | 0 | (0) | 0 |
| 15 | 12544 | 9 | (4) | 0.1 | 5167 | 33 | (24) | 0.6 |
| 16 | 12336 | 12 | (3) | 0.1 | 5063 | 76 | (41) | 1.5 |

TABLE 10
Number of samples with different number of consistent sites across the 8 SNP sites.
Numbers in parentheses indicate those contributed by African populations.

| | | | | | | | Percentage | |
|---|---|---|---|---|---|---|---|---|
| SNP | | | | | | | of sites that | |
| agreement | SMN1 CN = 1 | CN = 2 | CN = 3 | CN = 4 | CN = no-call | Total | disagree | |
| 8 | 163 | 6325 | 594 | 111 | 0 | 7193 (475) | 0 | (0) |
| 7 | 52 | 3141 | 285 | 28 | 0 | 3506 (199) | 11.3 | (1.6) |
| 6 | 25 | 1197 | 150 | 9 | 0 | 1381 (137) | 16.3 | (6) |
| 5 | 9 | 356 | 86 | 6 | 1 | 458 (74) | 21.1 | (10) |
| <5 | 2* | 92* | 44* | 1* | 36 | 175 (26) | 19.6 | (6.9) |

*Calls are made in these samples based on the second majority rule (see Methods).

TABLE 11
Number of no-calls due to disagreement and discrepant
calls made with reduced number of sites.

| | | | | | |
|---|---|---|---|---|---|
| # sites for majority rule | 8 (Require | 6 (4) | 4 (3) | 2 (2) | 1 (c.840C) |
| | 5 to agree) | | | | (1) |
| # no-calls due to | 175 | 298 | 766 | 1149 | 700 |
| disagreement | | | | | |
| # calls different from those | 0 | 0 | 1 | 6 | 41 |
| made with using 8 sites | | | | | |

TABLE 12
Validation samples.

| | SMN copy number caller | | | |
|---|---|---|---|---|
| | intact | intact | Digital PCR | |
| Sample ID | SMN1 CN | SMN2 CN | SMN1 CN | SMN2 CN |
| NA03813 | 0 | 3 | 0 | 3 |
| NA09677 | 0 | 3 | 0 | 3 |
| NA23689 | 0 | 3 | 0 | 3 |
| NA00232 | 0 | 2 | 0 | 2 |
| NA10684 | 0 | 2 | 0 | 2 |
| NA23687 | 1 | 2 | 1 | 2 |
| NA23688 | 1 | 2 | 1 | 2 |
| NA03815 | 1 | 1 | 1 | 1 |
| MB122 | 2 | 0 | 2 | 0 |
| MB226 | 2 | 1 | 2 | 1 |
| MB119 | 3 | 1 | 3 | 1 |
| MB370 | 3 | 1 | 3 | 1 |
| MB489 | 0 | 2 | 0 | 2 |
| MB364 | 0 | 2 | 0 | 2 |
| MB691 | 0 | 2 | 0 | 2 |
| MB488 | 0 | 2 | 0 | 2 |
| MB219 | 0 | 2 | 0 | 2 |
| MB228 | 0 | 2 | 0 | 2 |
| MB501 | 0 | 2 | 0 | 2 |
| MB362 | 0 | 2 | 0 | 2 |
| MB692 | 0 | 2 | 0 | 2 |
| MB234 | 0 | 2 | 0 | 2 |
| MB693 | 0 | 2 | 0 | 2 |
| MB510 | 0 | 2 | 0 | 2 |
| MB114 | 0 | 2 | 0 | 2 |
| MB116 | 1 | 2 | 1 | 2 |
| MB115 | 1 | 2 | 1 | 2 |
| MB104 | 2 | 2 | 2 | 2 |
| MB384 | 2 | 2 | 2 | 2 |
| MB338 | 2 | 2 | 2 | 2 |
| MB344 | 2 | 2 | 2 | 2 |
| MB345 | 2 | 2 | 2 | 2 |
| MB349 | 2 | 2 | 2 | 2 |
| MB113 | 2 | 2 | 2 | 2 |
| MB366 | 2 | 2 | 2 | 2 |
| MB351 | 3 | 2 | 3 | 2 |
| MB355 | 0 | 3 | 0 | 3 |
| MB361 | 0 | 3 | 0 | 3 |
| MB378 | 0 | 3 | 0 | 3 |
| MB232 | 0 | 3 | 0 | 3 |
| MB106 | 0 | 3 | 0 | 3 |
| MB222 | 0 | 3 | 0 | 3 |
| MB509 | 0 | 3 | 0 | 2 |
| MB112 | 0 | 3 | 0 | 3 |
| MB339 | 1 | 3 | 1 | 3 |
| MB377 | 0 | 4 | 0 | 4 |
| MB356 | 0 | 4 | 0 | 4 |
| MB503 | 0 | 4 | 0 | 4 |

TABLE 13
SMN1, SMN2 and SMN* CN calls for 258 trios in the Next Generation Children project cohort.

| SMN1 | | | | | SMN2 | | | | | SMN* | | | | |
|---|---|---|---|---|---|---|---|---|---|---|---|---|---|---|
| Number of | Fa- | Moth- | Pro- | Pro- | Number of | Fa- | Moth- | Pro- | Pro- | Number of | Fa- | Moth- | Pro- | Pro- |
| families | ther | er | band1 | band2 | families | ther | er | band1 | band2 | families | ther | er | band1 | band2 |
| 207 | 2 | 2 | 2 | | 53 | 2 | 2 | 2 | | 174 | 0 | 0 | 0 | |
| 8 | 2 | 2 | 2 | 2 | 29 | 2 | 1 | 1 | | 20 | 0 | 1 | 0 | |
| 8 | 2 | 3 | 3 | | 27 | 1 | 2 | 2 | | 15 | 0 | 1 | 1 | |
| 8 | 3 | 2 | 2 | | 23 | 1 | 2 | 1 | | 15 | 1 | 0 | 0 | |
| 7 | 3 | 2 | 3 | | 23 | 2 | 1 | 2 | | 9 | 1 | 0 | 1 | |
| 4 | 2 | 3 | 2 | | 17 | 1 | 1 | 1 | | 6 | 0 | 0 | 0 | 0 |
| 3 | 1 | 2 | 1 | | 12 | 2 | 0 | 1 | | 4 | 1 | 1 | 1 | |
| 3 | 1 | 2 | 2 | | 11 | 1 | 1 | 2 | | 3 | 1 | 1 | 0 | |
| 2 | 1 | 1 | 0 | | 9 | 1 | 1 | 0 | | 2 | 0 | 2 | 1 | |
| 2 | 2 | 2 | 1 | | 7 | 0 | 1 | 1 | | 2 | 1 | 0 | 1 | 0 |
| 2 | 2 | 3 | 2 | 3 | 6 | 0 | 2 | 1 | | 2 | 1 | 0 | 1 | 1 |
| 2 | 3 | 3 | 3 | | 4 | 1 | 0 | 1 | | 2 | 2 | 1 | 1 | |
| 1 | 2 | 1 | 1 | | 3 | 0 | 0 | 0 | | 1 | 0 | 2 | 2 | |
| 1 | 2 | 2 | 3 | | 3 | 2 | 2 | 1 | | 1 | 1 | 1 | 2 | |
| | | | | | 2 | 1 | 2 | 1 | 1 | 1 | 2 | 0 | 1 | |
| | | | | | 2 | 1 | 2 | 2 | 2 | 1 | 3 | 0 | 2 | |
| | | | | | 2 | 1 | 3 | 1 | | | | | | |
| | | | | | 2 | 2 | 1 | 1 | 2 | | | | | |
| | | | | | 2 | 2 | 1 | 3 | | | | | | |
| | | | | | 2 | 2 | 2 | 1 | 3 | | | | | |
| | | | | | 2 | 2 | 2 | 2 | 2 | | | | | |
| | | | | | 2 | 2 | 2 | 3 | | | | | | |
| | | | | | 2 | 2 | 3 | 3 | | | | | | |
| | | | | | 2 | 3 | 2 | 3 | | | | | | |
| | | | | | 1 | 0 | 1 | 0 | | | | | | |
| | | | | | 1 | 1 | 0 | 0 | | | | | | |
| | | | | | 1 | 1 | 3 | 2 | | | | | | |
| | | | | | 1 | 1 | 4 | 3 | | | | | | |
| | | | | | 1 | 2 | 3 | 2 | | | | | | |
| | | | | | 1 | 2 | 4 | 4 | | | | | | |
| | | | | | 1 | 3 | 0 | 1 | | | | | | |
| | | | | | 1 | 3 | 1 | 2 | | | | | | |
| | | | | | 1 | 3 | 2 | 2 | | | | | | |
| | | | | | 1 | 3 | 2 | 4 | | | | | | |
| | | | | | 1 | 4 | 1 | 2 | | | | | | |

TABLE 14
Number of samples by population in
1 kGP and NIHR BioResource cohorts.

| | | NIHR BioResource | NIHR BioResource |
|---|---|---|---|
| | | (including NGC, | (including NGC, |
| Ethnicity | 1 kGP | unrelated) | total) |
| African | 661 | 253 | 295 |
| European | 503 | 9186 | 11652 |
| South Asian | 489 | 713 | 1012 |
| East Asian | 504 | 91 | 97 |
| Admixed- | 347 | 0 | 0 |
| American | | | |
| Other | 0 | 0 | 1127 |
| Total | 2504 | 10243 | 14183 |

TABLE 15
SMN1, SMN2 and SMN* copy number frequencies by population.

| | | SMN1 | | | | SMN2 | |
|---|---|---|---|---|---|---|---|
| Ethnicity | Total | 1 | 2 | 3 | 4 | 0 | 1 |
| African | 902 | 4 | 404 | 373 | 121 | 226 | 449 |
| | | (0.44%) | (44.79%) | (41.35%) | (13.41%) | (25.06%) | (49.78%) |
| European | 9648 | 212 | 8899 | 524 | 13 | 833 | 3850 |
| | | (2.2%) | (92.24%) | (5.43%) | (0.13%) | (8.63%) | (39.9%) |
| South- | 1199 | 20 | 965 | 195 | 19 | 78 | 400 |
| Asian | | (1.67%) | (80.48%) | (16.26%) | (1.58%) | (6.51%) | (33.39%) |
| East- | 593 | 8 | 552 | 33 | 0 | 28 | 211 |
| Asian | | (1.35%) | (93.09%) | (5.56%) | (0.0%) | (4.72%) | (35.58%) |
| Admixed- | 341 | 7 | 296 | 36 | 2 | 30 | 136 |
| American | | (2.05%) | (86.8%) | (10.56%) | (0.59%) | (8.8%) | (39.88%) |

| | SMN2 | SMN* | | | | |
|---|---|---|---|---|---|---|
| Ethnicity | 2 | 3 | 4 | 0 | 1 | 2 |
| African | 214 | 13 | 0 | 892 | 9 | 1 |
| | (23.73%) | (1.44%) | (0.0%) | (98.89%) | (1.0%) | (0.11%) |
| European | 4667 | 279 | 19 | 7591 | 1912 | 137 |
| | (48.37%) | (2.89%) | (0.2%) | (78.74%) | (19.83%) | (1.42%) |
| South- | 686 | 29 | 5 | 1155 | 40 | 0 |
| Asian | (57.26%) | (2.42%) | (0.42%) | (96.65%) | (3.35%) | (0.0%) |
| East- | 340 | 12 | 2 | 591 | 2 | 0 |
| Asian | (57.34%) | (2.02%) | (0.34%) | (99.66%) | (0.34%) | (0.0%) |
| Admixed- | 162 | 11 | 2 | 302 | 37 | 2 |
| American | (47.51%) | (3.23%) | (0.59%) | (88.56%) | (10.85%) | (0.59%) |

TABLE 16
Comparison of carrier calls made in the 1 kGP samples by this example and Larson et al.

| | | | | | | Carrier | |
|---|---|---|---|---|---|---|---|
| | | | | | | probability, | |
| | | | | | Called as | adjusted, | GS calls |
| | | | | | carrier in | by Larson | validated |
| Sample ID | Ethnicity | SMN1 CN | SMN2 CN | SMN* CN | Larson et al. | et al. | by MLPA |
| HG03583 | AFR | 1 | 1 | 0 | yes | 0.645 | yes |
| HG01205 | AMR | 1 | 1 | 0 | yes | 0.756 | |
| HG01892 | AMR | 1 | 1 | 0 | yes | 0.902 | yes |
| HG01801 | EAS | 1 | 1 | 0 | yes | 0.541 | |
| NA11932 | EUR | 1 | 1 | 0 | yes | 0.716 | |
| NA20760 | EUR | 1 | 1 | 0 | yes | 0.638 | yes |
| NA20896 | SAS | 1 | 1 | 0 | yes | 0.514 | yes |
| HG01948 | AMR | 1 | 2 | 0 | yes | 0.678 | yes |
| HG02265 | AMR | 1 | 2 | 0 | yes | 0.982 | |
| HG01085 | AMR | 1 | 2 | 0 | yes | 1 | |
| NA20812 | EUR | 1 | 2 | 0 | yes | 0.999 | yes |
| NA20764 | EUR | 1 | 2 | 0 | yes | 0.982 | yes |
| HG00324 | EUR | 1 | 2 | 0 | yes | 0.997 | yes |
| NA12383 | EUR | 1 | 2 | 0 | yes | 1 | |
| HG03953 | SAS | 1 | 2 | 0 | yes | 0.972 | |
| HG02771 | AFR | 1 | 3 | 0 | yes | 0.997 | |
| HG01893 | AMR | 1 | 3 | 0 | yes | 1 | |
| HG02079 | EAS | 1 | 3 | 0 | yes | 0.976 | |
| NA20814 | EUR | 1 | 3 | 0 | yes | 1 | |
| HG00281 | EUR | 1 | 3 | 0 | yes | 1 | yes |
| HG00346 | EUR | 1 | 3 | 0 | yes | 1 | yes |
| HG03740 | SAS | 1 | 3 | 0 | yes | 0.874 | |
| HG02087 | EAS | 1 | 4 | 0 | yes | 1 | |
| HG02134 | EAS | 1 | 4 | 0 | yes | 1 | |
| NA12778 | EUR | 1 | 4 | 0 | yes | 1 | |
| HG01773 | EUR | 1 | 4 | 0 | yes | 1 | yes |
| HG01492 | AMR | 2 | 2 | 0 | yes | 0.914 | |
| NA19723 | AMR | 2 | 2 | 0 | yes | 0.681 | |
| NA18542 | EAS | 2 | 2 | 0 | yes | 0.633 | |
| HG00525 | EAS | 2 | 2 | 0 | yes | 0.763 | yes |
| NA20792 | EUR | 2 | 2 | 0 | yes | 0.671 | yes |
| NA11843 | EUR | 2 | 2 | 0 | yes | 0.509 | |
| NA19711 | AFR | 2 | 3 | 0 | yes | 0.943 | |
| NA19346 | AFR | 2 | 3 | 0 | yes | 0.52 | yes |
| HG01248 | AMR | 2 | 4 | 0 | yes | 0.935 | |
| HG01094 | AMR | 2 | 4 | 0 | yes | 0.738 | |
| HG02156 | EAS | 1 | 0 | 0 | no | 2.36E−33 | |
| HG02180 | EAS | 1 | 1 | 0 | no | 7.26E−05 | |
| NA20790 | EUR | 1 | 1 | 0 | no | 0.489 | yes |
| NA20787 | EUR | 1 | 1 | 1 | no | 0.322 | yes |
| HG01686 | EUR | 1 | 1 | 1 | no | 0.00119 | yes |
| NA19456 | AFR | 1 | 2 | 0 | no | 0.278 | |
| HG01455 | AMR | 1 | 2 | 0 | no | 0.176 | |
| HG01863 | EAS | 1 | 2 | 0 | no | 0.42 | |
| HG01612 | EUR | 1 | 2 | 0 | no | 1.20E−07 | yes |
| NA20845 | SAS | 1 | 2 | 0 | no | 0.398 | |
| HG03928 | SAS | 1 | 2 | 0 | no | 0.442 | yes |

TABLE 17
Maximum likelihood estimates for percentage of singleton
and two-copy SMN1 alleles carrying g.27134T > G.

| Ethnicity | Singleton SMN1 allele | two-copy SMN1 allele |
|---|---|---|
| African | 18.4% | 78.5% |
| European | 0.02% *(1 kGP | 4.35% *(1 kGP |
| | European: 0.11%) | European: 10.0%) |
| South Asian | 0.05% | 2.54% |
| East Asian | 0.09% | 2.94% |
| Admixed-American | 1.2% | 24.5% |

*The NIHR BioResource cohort, which takes up the majority of the European population analyzed in this example due to its large sample size, includes Northern European samples that carry a lower frequency of g.27134T > G SNP than the more diverse European samples from the 1000 Genomes project.

TABLE 18
SMA carrier detection and residual risk estimates from this example,

| | | | | | This Example | | Luo et al.^{c} |
|---|---|---|---|---|---|---|---|
| | | | | | Residual | Residual | Residual |
| | | | Residual | Detection | risk | risk | risk |
| | Carrier | Detection | risk | rate | (CN = 2, | (CN = 2, | (CN = 2, |
| Ethnicity | frequency^{a} | rate (CN)^{a} | (CN = 2) | (CN + SNP) | SNP−) | SNP+) | SNP−) |
| African | 1 in 72 | 70.5% | 1 in 129 | 91.8% | 1 in 346 | 1 in 58 | 1 in 396 |
| | | | | | | | (African |
| | | | | | | | American) |
| European | 1 in 47 | 94.8% | 1 in 790 | 95.0% | 1 in 814 | 1 in 12 | 1 in 770 |
| | | | | | (1 kGP | (1 kGP | |
| | | | | | European | European | |
| | | | | | 1 in 846) | 1 in 27) | |
| Asian^{b} | 1 in 59 | 93.3% | 1 in 767 | 93.4% | 1 in 779 | 1 in 57 | 1 in 702 |
| Admixed- | 1 in 68 | 90.0% | 1 in 559 | 91.9% | 1 in 674 | 1 in 71 | 1 in 1762 |
| American | | | | | | | (Hispanic) |

| | Luo et al.^{c} | Feng et al.^{d} | | Alias et al.^{e} | |
|---|---|---|---|---|---|
| | Residual | Residual | Residual | Residual | Residual |
| | risk | risk | risk | risk | risk |
| | (CN = 2, | (CN = 2, | (CN = 2, | (CN = 2, | (CN = 2, |
| Ethnicity | SNP+) | SNP−) | SNP+) | SNP−) | SNP+) |
| African | 1 in 34 | 1 in 375 | 1 in 39 | NA | NA |
| | | (African | | | |
| | | American) | | | |
| European | 1 in 29 | 1 in 921 | 1 in 69 | 1 in 888 | ~1 |
| | | | | (Spanish) | |
| Asian^{b} | ~1 | 1 in 907 | 1 in 61 | NA | NA |
| Admixed- | 1 in 140 | 1 in 906 | 1 in 99 | NA | NA |
| American | | (Hispanic) | | | |

^{a}Numbers and SMN1 allele frequencies for residual risk calculation taken from Sugarman et al. Pan-ethnic carrier screening and prenatal diagnosis for spinal muscular atrophy: clinical laboratory analysis of >72 400 specimens. Eur J Hum Genet. 2012; 20(1): 27-32. doi: 10.1038/ejhg.2011.134
^{b}Includes East and South Asians
^{c}Luo et al. An Ashkenazi Jewish SMN1 haplotype specific to duplication alleles improves pan-ethnic carrier screening for spinal muscular atrophy. Genet Med Off J Am Coll Med Genet. 2014; 16(2): 149-156. doi: 10.1038/gim.2013.84
^{d}Feng et al. The next generation of population-based spinal muscular atrophy carrier screening: comprehensive pan-ethnic SMN1 copy-number and sequence variant analysis by massively parallel sequencing. Genet Med Off J Am Coll Med Genet. 2017; 19(8): 936-944. doi: 10.1038/gim.2016.215
^{e}Alias et al. Utility of two SMN1 variants to improve spinal muscular atrophy carrier diagnosis and genetic counselling. Eur J Hum Genet. 2018; 26(10): 1554. doi: 10.1038/s41431-018-0193-4

TABLE 19
SMN1/SMN2/SMN2Δ7-8 CNs in 1kGP and NIHR cohorts

| | 1kGP | | | | | NIHR | | | | | |
|---|---|---|---|---|---|---|---|---|---|---|---|
| | | SMN | | | | | SMN1 | | | | Kolmogorov- |
| | Total | CN = 1 | 2 | 3 | 4 | Total | CN = 1 | 2 | 3 | 4 | Smirnov test |
| EUR | 503 | 15 | 463 | 25 | 0 | 9145 | 197 | 8436 | 499 | 13 | 1 |
| | | 2.98% | 92.05% | 4.97% | 0.00% | | 2.15% | 92.25% | 5.46% | 0.14% | |
| EAS | 502 | 7 | 470 | 25 | 0 | 91 | 1 | 82 | 8 | 0 | 0.9999 |
| | | 1.39% | 93.63% | 4.98% | 0.00% | | 1.10% | 90.11% | 8.79% | 0.00% | |
| AFR | 653 | 3 | 293 | 261 | 96 | 249 | 1 | 111 | 112 | 25 | 0.8284 |
| | | 0.46% | 44.87% | 39.97% | 14.70% | | 0.40% | 44.58% | 44.98% | 10.04% | |
| SAS | 489 | 5 | 397 | 77 | 10 | 710 | 15 | 568 | 118 | 9 | 1 |
| | | 1.02% | 81.19% | 15.75% | 2.04% | | 2.11% | 80.00% | 16.62% | 1.27% | |

### Discussion

Due to the high sequence homology between SMN1 and SMN2, the SMN region is difficult to resolve with both short and long read sequencing and thus far this important region has been excluded from standard WGS analysis. This example demonstrates a method that can resolve the CNs of SMN1 and SMN2 independently using short-read WGS data, filling in an important gap in SMA diagnosis and carrier screening for precision medicine initiatives. Accurate measurement of SMN1 and SMN2 CNs is important not only for the diagnosis of SMA but also is a prognostic indicator and the basis of therapeutic options. SMN2 CN has been used as a criterion for many clinical trials for SMA, including Nusinersen and Zolgensma.

As a demonstration of this method, CN calls for SMN1 and SMN2 were made using sequencing data from 12,747 samples covering five distinct subpopulations. The following were identified: 251 samples with whole gene losses (less than two copies) and 1317 with whole gene gains (more than two copies) of SMN1; 6241 samples with whole gene losses and 1274 with whole gene gains of SMN2; 2144 samples carrying one or more copies of the truncated form SMN*. The role that deletions, duplications or gene conversion play to drive the CN changes in this region cannot be accurately quantified. Evidence supporting all three mechanisms includes: 1) 3853 samples with total (SMN1+SMN2) CN<4 (deletions), 2) 670 samples with total CN>4 (duplications) and 3) a strong inverse correlation between the SMN1 and SMN2 CN (gene conversion, FIG. 14C). Additionally, a carrier frequency between 1:42 and 1:101, depending on ancestral population, were identified (Table 7). The CN frequencies by population were highly different, and the per-population results of this example agree with previous population studies. While the agreement provides qualitative support for the accuracy of the method, the accuracy of the method was directly assessed by comparing the CN calls made by the method against the results from digital PCR. In this direct comparison, all ( 48/48) of the SMN1 and 98% ( 47/48) of the SMN2 CN calls by the method agree with the digital PCR-based results. The one disagreement was due to a 2 kb deletion that was not targeted by the method and, importantly, the method properly identified the SMA status of this sample.

In this example, the CN calling was optimized to work for individuals of any ancestry and thus limited SMN1/2 differentiation to the functionally important splice variant plus seven sites in high concordance with the splice variant across all populations (FIG. 14A). By quantifying the concordance between all of the reference differences and the splice variant, the method was able to identify variations in these fixed differences that, if not accounted for properly (e.g., removed from our analysis) could lead to errors in our CN calls. Not accounting for fixed differences would be especially problematic in analyzing Africans because they harbor more diverse haplotypes. Population genetic studies, for example including using long read sequencing, can help profile the haplotypic diversity across populations more directly and identify new variant sites that could further improve the accuracy of SMN1/SMN2 differentiation.

One type of “silent” carrier occurs when an individual has two copies of the SMN1 gene but they are both on the same haplotype. A SNP (g.24134T>G) has been used to identify individuals that are at an increased risk of being carriers when SMN1 CN is two but the risk associated with this SNP can vary greatly between studies and populations (Table 17). When an individual has just one copy of SMN1, the individual can be definitively identified as a carrier, but this variant only indicates a 2-8% chance of being a carrier when SMN1 CN is two. With WGS, cataloging the different variants that occur with different CN combinations of SMN1 and SMN2 can be possible and identifying additional markers that could be used to improve our ability to identify these “silent” carriers can be possible. In addition, the loss of the c.840C>T splice variant currently explains around 95% of SMA cases and the remaining cases include other pathogenic variants. These other pathogenic variants represent another type of “silent” carrier. The method can directly genotype these other pathogenic variants as part of the testing process, further improving the ability to detect SMA carriers and cases.

While there exist difficult regions in the genome where normal WGS pipelines do not deliver variant calls, this example demonstrates the ability to apply WGS paired with a targeted bioinformatics approach to solve one such difficult region. This targeted strategy (WGS+specialized bioinformatics) can be applied to a number of difficult variants, such as repeat expansions and CYP2D6 disclosed herein. Traditionally, performing all of the known genetic tests and carrier screening on every individual was cost effective, so candidates for specific genetic testing were identified using information such as the carrier rate and family history. However, this process means that many people without a family history who would benefit from knowing their SMA status did not routinely have access to this data. Once WGS analysis can detect all SNVs and CNVs in all clinically relevant genes accurately then a more general and population-wide genetic testing strategy can be feasible with a single test. Improving WGS to become economical as a substitute for one current genetic test will help facilitate the integration of more genetic tests and carrier screens into WGS, allowing more general access to genetic testing population-wide. WGS provides a valuable opportunity to assess the entire genome for genetic variation and the continued development of more targeted bioinformatics solutions for difficult regions with WGS data will help bring the promise of personalized medicine one step closer to a reality.

### Example 2

### Accurate CYP2D6 Genotyping Using Whole Genome Sequencing Data

This Example and Appendix A describe genotyping CYP2D6 using whole genome sequencing data. The content of Appendix A is incorporated herein by reference in its entirety.

CYP2D6 is involved in the metabolism of 25% of all drugs and is a key target for personalized medicine. Genotyping CYP2D6 is challenging due to its high polymorphism, the presence of common structural variants (SVs), and high sequence similarity with the gene's pseudogene paralog CYP2D7. Disclosed herein a bioinformatics method, also referred to herein as Cyrius. that can accurately genotype CYP2D6 using whole genome sequencing (WGS) data. The method had superior performance (97.9% concordant with truth) compared to other methods (85.6-88.8%) in 138 samples with GeT-RM consensus calls and 50 additional samples with Pacific Biosciences of California, Inc. (Menlo Park, Calif.), also known as PacBio, sequencing data. A particular differentiator of the method is the ability to call structural variant star alleles. The method correctly identified 97.2% ( 70/72) structural variant star alleles compared to 77.8-88.9% ( 56/72 and 64/72) for the other methods. Applying the method to 2504 samples from the 1000 Genomes Project (1kGP), that CYP2D6 star alleles involving SVs were estimated to be up to 32.2% more frequent than previously reported for some populations. This example provides benchmarking results against the largest validation dataset so far. In some embodiments, the method is a useful tool for pharmacogenomics applications with WGS. The method can help bring the promise of precision medicine one step closer to reality.

### Introduction

There is significant variation in the response of individuals to a large number of clinically prescribed drugs. A strong contributing factor to this differential drug response is the genetic composition of the drug-metabolizing genes. Precision medicine requires genotyping pharmacogenes to enable personalized treatment. Cytochrome P450 2D6 (CYP2D6) is one of the most important drug-metabolizing genes and is involved in the metabolism of 25% of drugs. The CYP2D6 gene is highly polymorphic, with 106 star alleles defined by the Pharmacogene Variation (PharmVar) Consortium (pharmvar.org/gene/CYP2D6). CYP2D6 star alleles are CYP2D6 gene copies defined by a combination of small variants (such as single nucleotide variations (SNVs) and insertions/deletions (indels)) and structural variants (SVs), and correspond to different levels of CYP2D6 enzymatic activity, such as poor, intermediate, normal, or ultrarapid metabolizer.

The genotyping of CYP2D6 is challenged by the presence of a nonfunctional paralog, CYP2D7, that is located upstream of CYP2D6 and shares 94% sequence similarity, with a few near-identical regions. Deletions and duplications of CYP2D6 and fusions between CYP2D6 and its pseudogene paralog, CYP2D7, are common. Traditionally, CYP2D6 genotyping has been done with arrays or polymerase chain reaction (PCR) based methods, such as TaqMan assays, droplet digital PCR (ddPCR) and long-range PCR. These assays differ in the number of star alleles (variants) they target, leading to variability in genotyping results across assays. A common limitation of these methods is that: 1) the wild-type allele *1 is often a default call when none of the targeted variants is detected, or 2) a parent allele, such as *2, is assigned when the variant defining the true star allele is not tested. These assays are low throughput and often have difficulty detecting structural variants.

Profiling the entire genome at high throughput and in a clinically relevant timeframe is possible with next-generation sequencing (NGS). Large scale population sequencing efforts are undertaken, and pharmacogenomics testing can be a desirable target. CYP2D6 genotyping with NGS is particularly challenging due to common gene conversions between CYP2D6 and CYP2D7 (referred to as CYP2D6/7 hereafter), common SVs (gene deletions, duplications and CYP2D6/7 fusion genes), as well as the sequence similarity between CYP2D/7, which results in ambiguous read alignments to either genes. Some existing callers cannot detect complex structural variants and have been shown to have low performance. Other existing callers, such Aldy (Numanagic et al. Allelic decomposition and exact genotyping of highly polymorphic and structurally variant genes. Nat Commun. 2018; 9(1):1-11. Doi:10.1038/s41467-018-03273-1) and Stargazer (Lee et al. Stargazer: a software tool for calling star alleles from next-generation sequencing data using CYP2D6 as a model. Genet Med. 2019; 21(2):361. Doi:10.1038/s41436-018-0054-0), rely on accurate read alignment of sequence reads to CYP2D6 in order to detect SVs based on depth and derive the haplotype configurations based on the observed small variants and SVs. However, accurate read alignment of sequence reads to CYP2D6 is often not possible at many positions throughout the gene as the sequence is highly similar with CYP2D7 or even indistinguishable due to gene conversion. As a result, depth patterns can be ambiguous, and the callers can make false positive/negative small variant calls. Some callers do not support hg38, so many studies will require a re-alignment to hg37 to use these tools.

The availability of a panel of reference samples by the CDC Genetic Testing Reference Material Program (GeT-RM; Gaedigk et al. Characterization of Reference Materials for Genetic Testing of CYP2D6 Alleles: A GeT-RM Collaborative Project. J Mol Diagn JMD. August 2019. Doi:10.1016/j.jmoldx.2019.06.007), where the consensus genotypes of major pharmacogenetic genes are derived using multiple genotyping platforms, has enabled assessment of genotyping accuracy for newly developed methods. GeT-RM covers 43 of the 106 CYP2D6 star alleles. Additionally, many of the single-marker methods used for those consensus genotypes can be error prone, leading to conflicts between methods. The availability of high-quality long reads can provide a complete picture of CYP2D6 for improved validation of complicated variants and haplotypes. Disclosed herein is Cyrius, a WGS-based CYP2D6 genotyping method that overcomes the challenges with CYP2D6 and CYP2D7 (referred to herein as CYP2D6/7). Cyrius has a superior genotyping accuracy over Aldy and Stargazer in 138 GeT-RM reference samples and 50 samples with whole genome PacBio sequencing data, covering 41 of 106 known star alleles. The method was applied to high depth sequence data on 2504 unrelated samples from the 1000 Genomes Project (1kGP) to report on the distribution of star alleles across five ethnic populations. This analysis demonstrates differences with frequencies in PharmGKB, highlighting the potential errors associated with merging limited star allele calls made using diverse technologies designed to identify specific subsets of the known star alleles. This analysis expands the current understanding on CYP2D6's genetic diversity, particularly on complex star alleles with SVs.

### Materials and Methods

Samples

The following were analyzed: WGS data for 138 GeT-RM reference samples, including 96 samples that were genotyped in the initial GeT-RM study and updated in the latest GeT-RM release, as well as 42 additional samples that were newly added in the latest GeT-RM release. For the first batch of 96 samples, WGS was performed with TruSeq DNA PCR-free sample preparation with 150 bp paired reads sequenced on Illumina, Inc. (San Diego, Calif.) HiSeq X instruments. Genome build GRCh37 was used for read alignment. Sequence data for 70 of these samples was downloaded from ebi.ac.uk/ena/data/view/PRJEB19931. The WGS data for the second batch of 42 samples were downloaded from NYGC as part of the 1000 Genomes Project (see below).

For population studies, the 1000 Genomes Project (1kGP) data, for which WGS BAMs for 2504 samples were downloaded from ncbi.nlm.nih.gov/bioproject/PRJEB31736/, was used. These BAM files were generated by sequencing 2×150 bp reads on Illumina NovaSeq 6000 instruments from PCR-free libraries and aligning them to the human reference, hs38DH. WGS data for 70 GeT-RM samples were downloaded from ebi.ac.uk/ena/data/view/PRJEB19931.

PacBio Sequencing

The gDNA samples were purchased from Coriell Institute for Medical Research (Coriell, NJ, USA). The quality of gDNA samples was evaluated by Nanodrop (ThermoFisher, MA, USA). The A280/A260 ratio need to be in the range of 1.8-2.0 and A260/230 ratio is ≥2.0. The molecular weight of the gDNA was evaluated by Femto Pulse System (Agilent CA, USA). The majority of the DNA fragments size should be >40 kb. If the quality of gDNA sample from Coriell is lower than the protocol requirement, fresh DNA was extracted from the B-lymphocyte cell (Coriell, NJ, USA) with Qiagen DNA extraction kit (Qiagen, CA, USA).

10 ug gDNA was fragmented to 15 kb using Covaris g-tubes following manufacturer's instruction (Covaris, MA, USA). DNA was purified using 0.45× AMPure XP beads (Beckman Coulter, IN, USA) according to manufacturer's instructions. The sheared DNA size was confirmed by Femto Pulse System (Agilent CA, USA).

Libraries were constructed following PacBio “Preparing HiFi SMRTbell® Libraries using SMRTbell Template Prep Kit 1.0” or “HiFi SMRTbell® Libraries using SMRTbell Express Template Prep Kit 2.0” protocol (PacBio CA, USA). The library size selected for 15˜20 kb using a Sage Elf instrument with 0.75% agarose (Sage Science, MA, USA). Quality control of all libraries was performed with Qubit (Life Technologies, CA, USA) and Femto Pulse (Agilent, CA, USA).

The PacBio Sequel II Sequencing Platform was Used to Sequence. 20× Coverage WGS Data Generally Obtained from 2˜3 SMRT Cells (Pacific Biosciences, CA, USA).CYP2D6 Genotyping Method

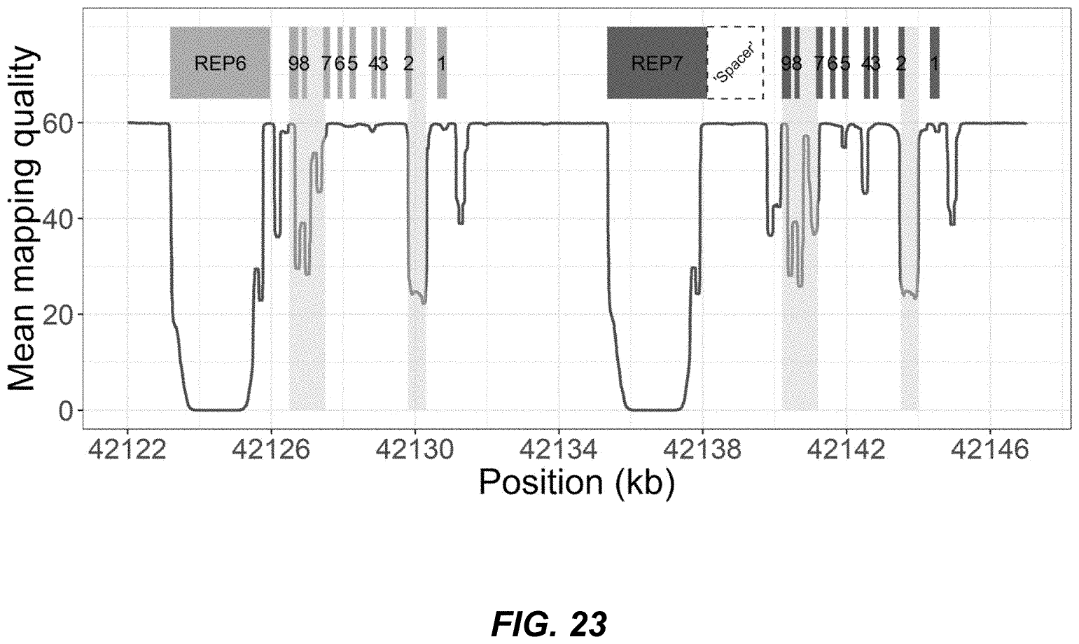

The method described in this example, Cyrius, first calls the sum of the copy number (CN) of CYP2D6/7, following a similar method as described in Example 1. Read counts were calculated directly from the WGS aligned BAM file using all reads mapped to either CYP2D6 or CYP2D7, including those with a mapping quality of zero to account for regions with high sequence homology. The summed read count was normalized by region length. GC correction was then performed against 3000 pre-selected 2 kb regions across the genome. These 3000 normalization regions were randomly selected from the genome for stable coverage across population samples to infer the sequencing depth and capture GC bias. The normalized depth values across the population were modeled with a one-dimensional mixture of 11 Gaussians with constrained means that center around each integer CN value representing CN states ranging from 0 to 10. CNs of CYP2D6+CYP2D7 were called from the Gaussian mixture model (GMM) with a posterior probability threshold of 0.95. The same approach was used to call the CN of the 1.5 kb spacer region between the repeat REP7 and CYP2D7 to infer the CN of REP7-containing fusion genes (FIG. 23).

FIG. 23 is a non-limiting exemplary plot showing WGS data quality in CYP2D6/7 region. Mean mapping quality across 1kGP samples are plotted for each position in the CYP2D6/7 region. A median filter is applied in a 200 bp window. REP6, REP7, and the 9 exons of CYP2D6/7 are shown as boxes on the left (CYP2D6) and right (CYP2D7). Two 2.8 kb repeat regions downstream of CYP2D6 (REP6) and CYP2D7 (REP7) are identical and essentially unalignable. The dotted box denotes the spacer region between CYP2D7 and REP7. Two major homology regions within the genes are shaded

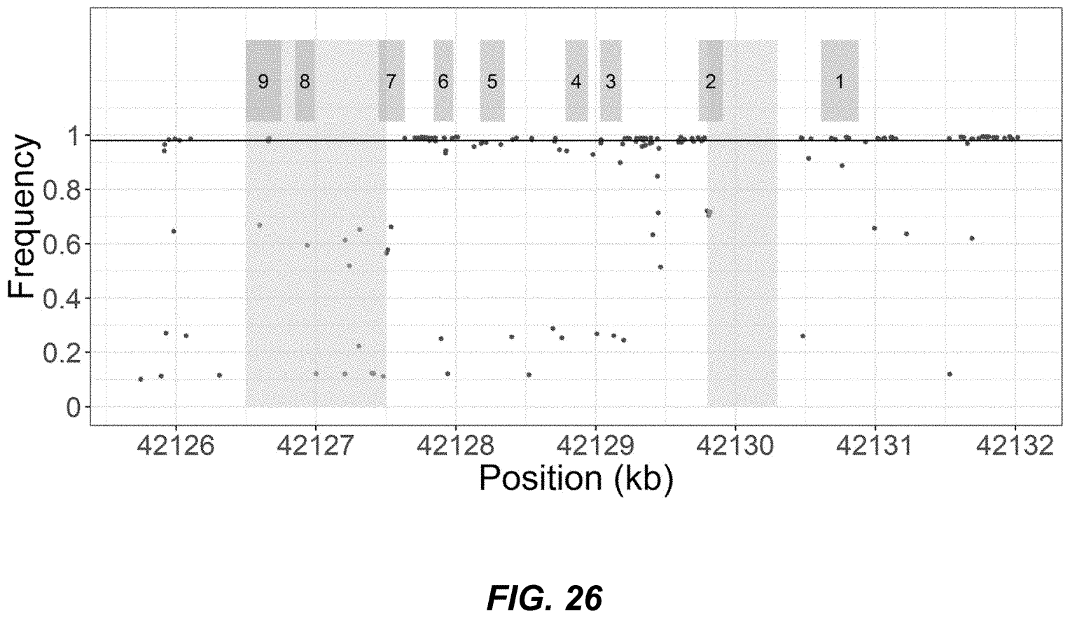

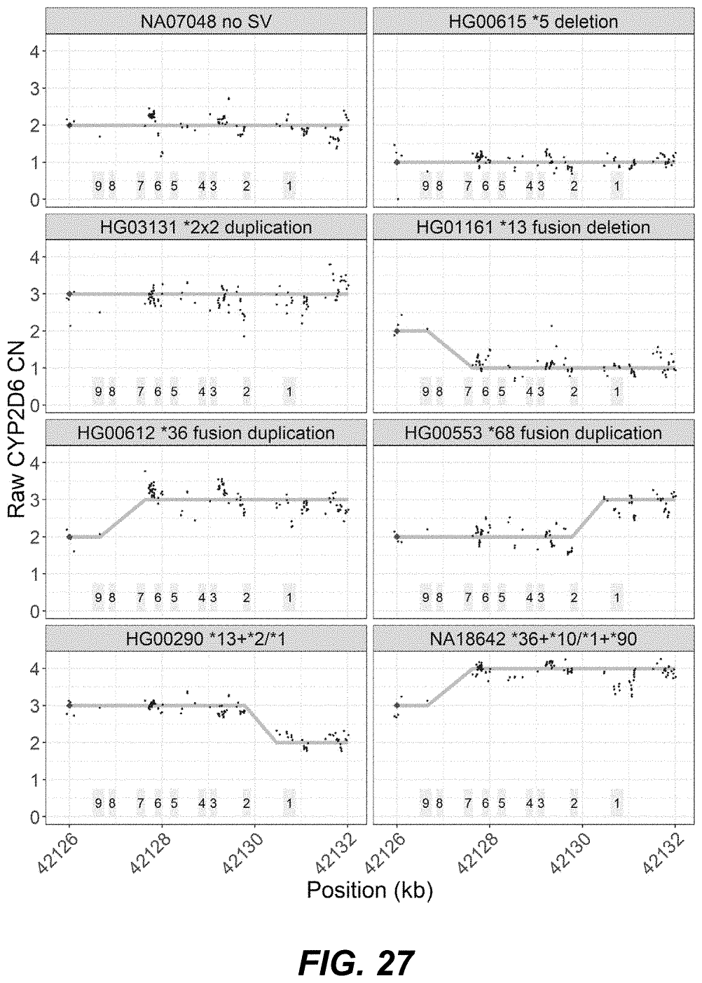

The method identified 118 CYP2D6/CYP2D7 differentiating bases (see the Additional Information of this example, FIG. 26). At each of these differentiating base positions, Cyrius called the number of chromosomes carrying CYP2D6 and those carrying CYP2D7 by combining the total CYP2D6+CYP2D7 CN with the read counts supporting each of the gene-specific bases. Based on the called total CN, Cyrius iterated through all possible combinations of CYP2D6 CNs and CYP2D7 CNs and derived the combination that produces the highest posterior probability for the observed number of CYP2D6- and CYP2D7-supporting reads. Gene fusions were called by identifying the bases when the CN of CYP2D6 changed within the gene (FIG. 27).

Cyrius parsed the read alignments to identify small variants that define star alleles. Variants of interest were divided into those that fall into CYP2D6/CYP2D7 homology regions (i.e. the low mapping quality regions on FIG. 23) and variants that occur in unique regions of CYP2D6. For the former, Cyrius looked for variant reads in CYP2D6 and its corresponding site in CYP2D7. For the latter, Cyrius used the reads aligned to CYP2D6. CNs called in the region were also taken into account during small variant calling. For example, in a sample where a *68 duplication fusion had been identified, one haplotype should have an intact copy of CYP2D6 plus a copy of *68, and the other haplotype should have an intact copy of CYP2D6, and consequently, the CYP2D6 CN should be 3 upstream of Exon 2 and 2 downstream of Exon 2.

Finally Cyrius matched the called structural variants and small variants against the definition of star alleles (downloaded and parsed from PharmVar, pharmvar.org/gene/CYP2D6, last accessed in March 2019) to call star alleles, which were further grouped into haplotypes when, for example, there were more than two copies of CYP2D6. For this prior information was included to define the exact haplotypes, e.g., *68 was on the same haplotype as *4, and *36 was on the same haplotype as *10). These priors were made based on the tandem arrangement patterns described in PharmVar and are also supported by our truth data ( 12/12 for *68 and 25/25 for *36). An option was available to only match called structural variants and small variants against star alleles with known functions.

Of the 131 star alleles defined in PharmVar (last accessed in March 2020), 25 are still awaiting curation, so the example excluded those and focused on the 106 curated ones (another option is provided in Cyrius to include those uncurated ones). Of these 106 star alleles, four from our target list were removed, none of which are in GeT-RM. The removed star alleles include *61 and *63 (both unknown function), which are CYP2D6/7 hybrid genes very similar to *36 with the fusion breakpoint slightly upstream. Since it was not possible to distinguish the Exon7-Exon8 region between CYP2D6/7 (FIG. 26), these two star alleles cannot be distinguished from *36, and would be called as *36 by Cyrius. Additionally, *27 (normal function) and *32 (unknown function) were removed; *27 and *32 share g.42126938C>T, a gene conversion variant in a high homology region (a variant read will align to CYP2D7 perfectly. By counting CYP2D6- and CYP2D7-supporting reads at a single site, accurately distinguish 1 copy of CYP2D6 and 3 copies of CYP2D7 from 2 copies each 20 may be difficult. As a result, *27 would be called as *1 and *32 will be called as *41.

Validating Against Truth from GeT-RM and Long Reads

When comparing the CYP2D6 calls made by Cyrius, Aldy and Stargazer against the consensus genotypes provided by GeT-RM, a genotype was considered a match as long as all of the star alleles in the truth genotype are present, even if the haplotype assignment was different. An example of this occurs in several samples listed by GeT-RM as *1/*10+*36+*36 but called by Aldy as *1+*36/*10+*36.

When validating genotype calls against the PacBio data, PacBio reads that covered the entire CYP2D6 gene were analyzed to identify small variants known to define the star alleles. Long (˜10 kb) reads allow fully phasing these variants into haplotypes and these haplotypes were matched against the star allele table to determine which star allele each read represented. Reads carrying structural variation were determined by aligning reads against a set of reference contigs that were constructed to represent known structural variants (*51*13/*36/*68/duplications).

Running Aldy and Stargazer

Aldy v2.2.5 was run using the command “aldy genotype -p illumina -g CYP2D6”.

Stargazer v1.0.7 was run to genotype CYP2D6 using VDR as the control gene, with GDF and VCF files as input.

As Aldy and Stargazer only supported GRCh37, for 1kGP samples that were originally aligned against hs38DH, and realignment against GRCh37 was done using Isaac.

### Results

Validation and Performance Comparison

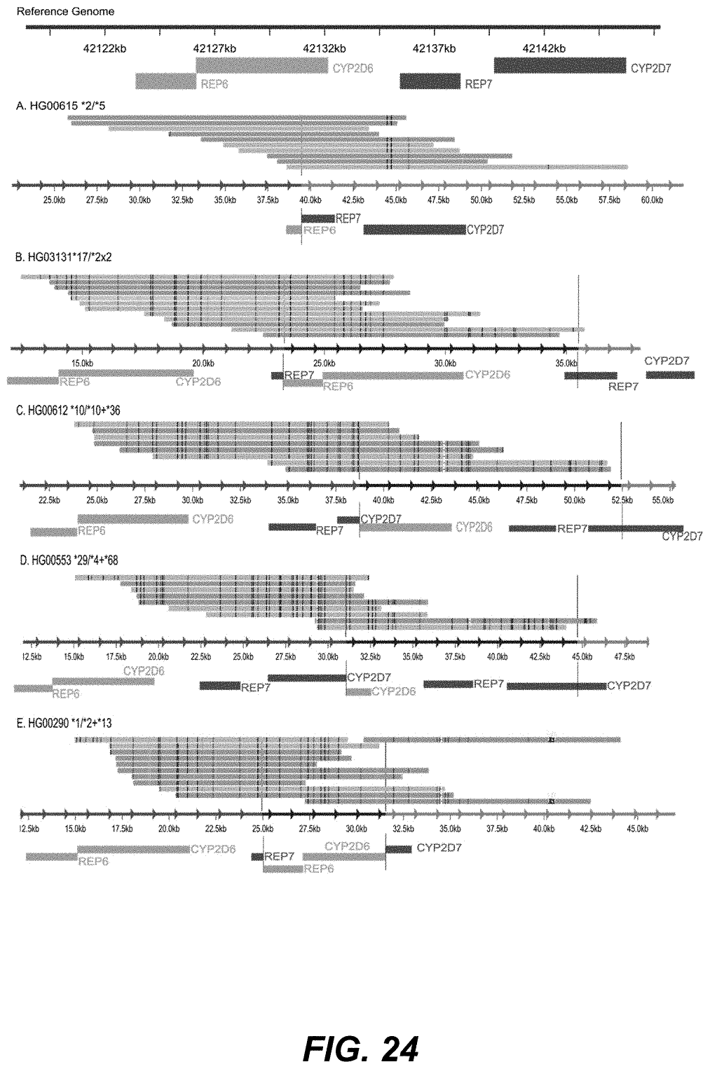

The CYP2D6 calls made by Cyrius, Aldy and Stargazer against 188 samples where high quality ground truth was obtained. These 188 samples included 138 GeT-RM samples and 50 samples with truth from PacBio whole genome sequencing were compared (Table 20, Table 21). The PacBio CCS data allowed locating and visualizing breakpoints of the common and rare structural variants in the region (FIG. 24) and thus served as a valuable resource for studying complex star alleles and confirmed the phasing of the variants for the star alleles. With short reads, these samples with SVs showed distinct depth signals that allowed calling SVs accurately (FIG. 27).

TABLE 20
Summary of benchmarking results against truth.

| | | | | | | | | | | Sensitivity, | Sensitivity, |
|---|---|---|---|---|---|---|---|---|---|---|---|
| | Total | Total | | Total | | Deletion | Duplication | Fusion | No SV | samples | samples |
| Caller | GeTRM | PacBio | Total | concordant | Sensitivity | N = 18 | N = 14 | N = 40 | N = 116 | with SV | without SV |
| Cyrius | 138 | 50 | 188 | 184* | 97.9% | 17 | 14 | 39 | 114 | 97.2% | 98.3% |
| Aldy | | | | 167 | 88.8% | 16 | 12 | 36 | 103 | 88.9% | 88.8% |
| Stargazer | | | | 161 | 85.6% | 17 | 11 | 28 | 105 | 77.8% | 90.5% |

*Improvements to Cyrius were made after seeing three discordant samples and Cyrius was then able to call 187 out of 188 of these samples accurately.

TABLE 21
Cyrius/Aldy/Stargazer results against GeT-RM and PacBio truth.

| | | | | | Truth | | |
|---|---|---|---|---|---|---|---|
| SampleID | Truth | Cyrius | Aldy | Stargazer | source | 1 kGP | Type |
| HG00276 | *4/*5 | *4/*5 | *4/*5 | *4/*5 | GeT-RM | | deletion |
| NA10831 | *4/*5 | *4/*5 | *4/*5 | *4/*5 | GeT-RM | | deletion |
| NA12873 | *1/*5 | *1/*5 | exited with | *1/*5 | GeT-RM | | deletion |
| | | | error | | | | |
| NA17235 | *1/*5 | *1/*5 | *1/*5 | *1/*5 | GeT-RM | | deletion |
| NA18855 | *1/*5 | *1/*5 | *1/*5 | *1/*5 | GeT-RM | | deletion |
| NA18868 | *2/*5 | *2/*5 | *2/*5 | *2/*5 | GeT-RM | | deletion |
| HG01706 | *1/*5 | *1/*5 | *1/*5 | *1/*5 | PacBio | | deletion |
| HG00615 | *2/*5 | *2/*5 | *2/*5 | *2/*5 | PacBio | | deletion |
| HG02523 | *1/*5 | *1/*5 | *1/*5 | *1/*5 | PacBio | | deletion |
| NA18992 | *1/*5 | *1/*5 | *1/*5 | *1/*5 | GeT-RM | | deletion |
| NA18861 | *5/*29 | *29/*5 | *29/*5 | *5/*29 | GeT-RM | | deletion |
| NA19035 | *2/*5 | *2/*5 | *2/*5 | *2/*5 | GeT-RM | 1 kGP | deletion |
| NA18945 | *1/*5 | *1/*5 | *1/*5 | *1/*5 | GeT-RM | 1 kGP | deletion |
| HG03225 | *5/*56 | *10/*5 | *5/*56 | *5/*56 | GeT-RM | 1 kGP | deletion |
| | | (updated to | | | | | |
| | | *56/*5) | | | | | |
| HG03259 | *5/*106 | *106/*5 | *106/*5 | *5/*106 | GeT-RM | 1 kGP | deletion |
| HG03246 | *5/*43 | *43/*5 | exited with | *5/*43 | GeT-RM | 1 kGP | deletion |
| | | | error | | | | |
| NA19317 | *5/*5 | *5/*5 | *5/*5 | *2/*2 | GeT-RM | 1 kGP | deletion |
| NA18873 | *5/*17 | *17/*5 | *17/*5 | *5/*17 | GeT-RM | 1 kGP | deletion |
| HG00436 | *2 × 2/*71 | *2 × 2/*71 | *2 + *2/*71 | *2 × 2/*71 | GeT-RM | | duplication |
| NA07439 | *4 × 2/*41 | *41/*4 × 2 | *4 + *4/*41 | *4 × 2/*41 | GeT-RM | | duplication |
| NA17244 | *2 × 2/ | *2 × 2/ | *4 + *4/ | *2 × 2/ | GeT-RM | | duplication |
| | *4 × 2 | *4 × 2 | *63 + *78 + | *4 × 2 | | | |
| | (+hybrid) | | *2 | | | | |
| NA19226 | *2/*2 × 2 | *2/*2 × 2 | *2/*2 + *2 | *2/*2 × 2 | GeT-RM | | duplication |
| NA24027 | *2 × 2/*6 | *2 × 2/*6 | *2 + *2/*6 | *1 × 2/*6 | GeT-RM | | duplication |
| NA19920 | *1/*4 × 2 | *1/*4 × 2 | *1/*4 + *4 | *1/*4 × 2 | GeT-RM | | duplication |
| NA19819 | *2/*4 × 2 | *2/*4 × 2 | *2/*4 + *4 | *2/*4 × 2 | GeT-RM | | duplication |
| NA19207 | *2 × 2/*10 | *10/*2 × 2 | *10/*2 + *2 | *2D × 2/*10 | GeT-RM | | duplication |
| NA17454 | *1 × 2/ | *1 × 2/ | *1 + *34/ | *1 × 2/ | GeT-RM | | duplication |
| | *2 × 2 | *2 × 2 | *2 + *2 | *2 × 2 | | | |
| NA19109 | *2 × 2/*29 | *2 × 2/*29 | *2 + *2/*29 | *2 × 2/*29 | GeT-RM | | duplication |
| NA15245 | *4 × 2/*4 | *4/*4 × 2 | *4/*4 + *4 | *4/*4 × 2 | GeT-RM | | duplication |
| HG00337 | *2 × 2/*22 | *2 × 2/*22 | *2 + *2/*22 | *1/*2 × 2 | GeT-RM | 1 kGP | duplication |
| HG01622 | *1/*2 × 2 | *1/*2 × 2 | *1/*2 + *2 | *1/*34 × 2 | PacBio | | duplication |
| HG03131 | *17/*2 × 2 | *17/*2 × 2 | *17/*2 + *2 | *2D × 2/*17 | PacBio | | duplication |
| HG01190 | *68 + *4/*5 | *5/*4 + *68 | *4/*68 | *5/*68 + *4 | GeT-RM | | fusion |
| NA12878 | *3/*4 + *68 | *3/*4 + *68 | *3/*4 + *68 | *3/*4 + *68 | PacBio | | fusion |
| NA12877 | *4/*4 + *68 | *4/*4 + *68 | *4/*4 + *68 | *4/*4 + *68 | PacBio | | fusion |
| NA21781 | *2 × 2 | *2 × 2 | *2 + *2/ | *2 × 2 | GeT-RM | | fusion |
| | *68 + *4 | *4 + *68 | *68 + *4 | *68 + *4 | | | |
| HG01772 | *4 + *68 | *4 + *68/ | *68 + *4/ | ungenotyped | PacBio | | fusion |
| | *4 + *68 | *4 + *68 | *68 + *4 | | | | |
| NA1832 | *1/(*68) + *4 | *1/*4 + *68 | *1/*68 + *4 | *1/*68 + *4 | GeT-RM | | fusion |
| NA12878 | *3/(*68) + *4 | *3/*4 + *68 | *3/*68 + *4 | *3/*68 + *4 | GeT-RM | 1 kGP | fusion |
| NA12154 | (*68) + *4/*33 | *33/*4 + *68 | *33/*68 + *4 | *33 × 2/ | GeT-RM | 1 kGP | fusion |
| | | | | *68 + *4 | | | |
| HG00731 | *4/*4 + *68 | *4/*4 + *68 | *4/*68 + *4 | *4/*68 + *4 | PacBio | 1 kGP | fusion |
| HG00553 | *29/*4 + *68 | *29/*4 + *68 | *29/*68 + *4 | *29/*68 + *4 | PacBio | 1 kGP | fusion |
| NA23874 | *4/*4 | *4/*4 + *68 | *4/*68 + *4 | *4/*68 + *4 | GeT-RM | | fusion |
| NA24008 | *1/*4 | *1/*4 + *68 | *1/*68 + *4 | *1/*68 + *4 | GeT-RM | | fusion |
| NA18524 | *1/*36 × 2 + | *1/*10 + *36 + | *1 + *36/ | *36 + *10/ | GeT-RM | | fusion |
| | *10 | *36 | *36 + *10 | *36 + *10 | | | |
| NA18526 | *1/*36 × 2 + | *1/*10 + *36 + | *1 + *36/ | *36 + *10/ | GeT-RM | | fusion |
| | *10 | *36 | *36 + *10 | *36 + *10 | | | |
| NA18540 | (*36 + )10/*41 | *41/*10 + *36 + | *36 + *10/ | *36 + *10/ | GeT-RM | | fusion |
| | | *36 | *61 + *69 | *36 + *10 | | | |
| NA18564 | *2A/*36 + *10 | *2/*10 + *36 | *2/*36 + *10 | *2/*36 + *10 | GeT-RM | | fusion |
| NA18565 | *10/*36 × 2 | *10/*10 + *36 | *10/*36 + *10 | *10/*36 + *10 | GeT-RM | | fusion |
| NA18617 | *36 + *10/ | *36 + *10/ | *36 + *10/ | *36 + *10/ | GeT-RM | | fusion |
| | *36 + *10 | *36 + *10 | *36 + *10 | *36 + *10 | | | |
| NA18959 | *2/*36 + *10 | *2/*10 + *36 | *2/*36 + *10 | *2/*36 + *10 | GeT-RM | | fusion |
| NA23246 | *10 × 2/ | *10 × 2/ | *10 + *10/ | *10 × 2/ | GeT-RM | | fusion |
| | *36 + *10 | *10 + *36 | *36 + *10 | *36 + *10 | | | |
| NA18980 | *2/*36 + *10 | *2/*10 + *36 | *2/*36 + *10 | *2/*36 + *10 | GeT-RM | | fusion |
| NA18642 | *36 + *10/ | *36 + *10/ | *1 + *90/ | *1 × 2/ | GeT-RM | 1 kGP | fusion |
| | *1 + *90 | *1 + *90 | *36 + *10 | *36 + *10 | | | |
| HG00463 | *36 + *10/ | *36 + *10/ | *36 + *10/ | *36 + *10/ | GeT-RM | 1 kGP | fusion |
| | *36 + *10 | *36 + *10 | *36 + *10 | *36 + *10 | | | |
| HG02373 | *14/*36 + *10 | *14/*10 + *36 | *14/*36 + *10 | *14/*36 + *10 | GeT-RM | 1 kGP | fusion |
| NA18572 | *36 + *10/*41 | *41/*10 + *36 | *10 + *83/*69 | *36 + *10/*41 | GeT-RM | 1 kGP | fusion |
| NA18632 | *36 × 2 + | *52/*10 + *36 + | *36 + *10/ | *36 + *10/ | GeT-RM | 1 kGP | fusion |
| | *10/*52 | *36 | *36 + *52 | *36 + *10 | | | |
| NA18563 | *1/*36 + *10 | *1/*10 + *36 | *1/*36 + *10 | *1/*36 + *10 | GeT-RM | 1 kGP | fusion |
| NA18545 | *5/*36 × 2 + | *36 + *10/ | *36 + *10/ | *36 + *10/ | GeT-RM | 1 kGP | fusion |
| | *10 × 2 | *36 + *10 | *36 + *10 | *36 + *10 | | | |
| HG02068 | *10/*10 + *36 | *10/*10 + *36 | *10/*36 + *10 | *10/*36 + *10 | PacBio | | fusion |
| HG00612 | *10/*10 + *36 | *10/*10 + *36 | *10/*36 + *10 | *10/*36 + *10 | PacBio | | fusion |
| HG00597 | *49/*10 + *36 + | *49/*10 + *36 + | *36 + *10 + | *36 + *10/ | PacBio | | fusion |
| | *36 + *83 | *36 + *83 | *49/*36 + *39 | *39 × 3 | | | |
| HG02015 | *10/*10 + *36 | *10/*10 + *36 | *10/*36 + *10 | *10/*36 + *10 | PacBio | | fusion |
| HG02071 | *2/*10 + *36 | *2/*10 + *36 | *2/*36 + *10 | *2/*36 + *10 | PacBio | | fusion |
| HG02129 | *1/*10 + *36 | *1/*10 + *36 | *1/*36 + *10 | *1/*36 + *10 | PacBio | | fusion |
| HG02074 | *10 + *36/ | *36 + *10/ | *36 + *10/ | *36 + *10/ | PacBio | | fusion |
| | *10 + *36 | *36 + *10 | *36 + *10 | *36 + *10 | | | |
| HG00844 | *10 + *36/ | *36 + *10/ | *36 + *10/ | *36 + *10/ | PacBio | 1 kGP | fusion |
| | *10 + *36 | *36 + *10 | *36 + *10 | *36 + *10 | | | |
| HG005 | *49/*10 + *36 | *49/*10 + *36 | *49/*10 + *36 | *10/*36 + *10 | PacBio | | fusion |
| (NA24631) | | | | | | | |
| NA19785 | *1/*13 + *2 | *2 + *13/*1 | *1/*79 + *2 | ungenotyped | GeT-RM | 1 kGP | fusion |
| HG00290 | *2 + *13/*1 | *2 + *13/*1 | *1/*79 + *2 | ungenotyped | PacBio | 1 kGP | fusion |
| HG00421 | *2/*10 × 2 | *10 + *36/*2 | *2/*36 + *10 | *2/*36 + *10 | GeT-RM, | 1 kGP | fusion, |
| | | (updated to | | | PacBio | | *10D is a |
| | | *10 × 2/*2) | | | | | fusion, see |
| | | | | | | | FIG. 27 |
| HG00589 | *1/*21 | *1/*21 | *1/*21 | *1/*21 | GeT-RM | | No SV |
| NA06991 | *1/*4 | *1/*4 | *1/*4 | *1/*4 | GeT-RM | | No SV |
| NA07000 | *2 (*35)/*9 | *35/*9 | *35/*9 | *9/*35 | GeT-RM | | No SV |
| NA07019 | *1/*4 | *1/*4 | *1/*4 | *1/*4 | GeT-RM | | No SV |
| NA07029 | *1/*35 | *1/*35 | *1/*35 | *1/*35 | GeT-RM | | No SV |
| NA07055 | *4/*4 | *4/*4 | *4/*4 | *4/*4 | GeT-RM | | No SV |
| NA07056 | *2/*4 | *2/*4 | *2/*4 | *2/*4 | GeT-RM | | No SV |
| NA07348 | *1/*6 | *1/*6 | *1/*6 | *1/*6 | GeT-RM | | No SV |
| NA07357 | *1/*6 | *1/*6 | *1/*6 | *1/*6 | GeT-RM | | No SV |
| NA10847 | *1/*41 | *1/*41 | *1/*41 | *1/*41 | GeT-RM | | No SV |
| NA10851 | *1/*4 | *1/*4 | *39/*4 | *1/*4 | GeT-RM | | No SV |
| NA10854 | *1/*4 | *1/*4 | *1/*4 | *1/*4 | GeT-RM | | No SV |
| NA11839 | *1/*2 | *1/*2 | *1/*2 | *1/*2 | GeT-RM | | No SV |
| NA11993 | *1/*9 | *1/*9 | *1/*9 | *1/*9 | GeT-RM | | No SV |
| NA12003 | *4/*35 | *35/*4 | *35/*4 | *4/*35 | GeT-RM | | No SV |
| NA12006 | *4/*41 | *41/*4 | *4/*41 | *4/*41 | GeT-RM | | No SV |
| NA12145 | *1/*4 | *1/*4 | *1/*4 | *1/*4 | GeT-RM | | No SV |
| NA12156 | *1/*4 | *1/*4 | *1/*4 | *1/*4 | GeT-RM | | No SV |
| NA12236 | *1/*4 | *1/*4 | *1/*4 | *1/*4 | GeT-RM | | No SV |
| NA12717 | *1/*1 | *1/*1 | *1/*1 | *1/*1 | GeT-RM | | No SV |
| NA12813 | *2/*4 | *2/*4 | *2/*4 | *2/*4 | GeT-RM | | No SV |
| NA17074 | *1/*2 | *1/*2 | *1/*2 | *1/*2 | GeT-RM | | No SV |
| NA17102 | *1/*40 | *1/*40 | *1/*40 | *1/*40 | GeT-RM | | No SV |
| NA17204 | *1/*35 | *1/*35 | *1/*35 | *1/*35 | GeT-RM | | No SV |
| NA17227 | *1/*9 | *1/*9 | *1/*9 | *1/*9 | GeT-RM | | No SV |
| NA17234 | *1/*41 | *1/*41 | *1/*41 | *1/*41 | GeT-RM | | No SV |
| NA17448 | *1/*28 | *1/*28 | *1/*28 | *1/*28 | GeT-RM | | No SV |
| NA17641 | *2/*35 | *2/*35 | *2/*35 | *2/*35 | GeT-RM | | No SV |
| NA17642 | *1/*1 | *1/*1 | *1/*61 | *1/*1 | GeT-RM | | No SV |
| NA17657 | *4/*9 | *4/*9 | *4/*9 | *4/*9 | GeT-RM | | No SV |
| NA17658 | *1/*2 | *1/*2 | *1/*2 | *1/*2 | GeT-RM | | No SV |
| NA17660 | *1/*2 | *1/*2 | *1/*2 | *1/*2 | GeT-RM | | No SV |
| NA17673 | *1/*4 | *1/*4 | *1/*4 | *1/*4 | GeT-RM | | No SV |
| NA17679 | *1/*4 | *1/*4 | *1/*4 | *1/*4 | GeT-RM | | No SV |
| NA17702 | *1/*35 | *1/*35 | *1/*35 | *1/*35 | GeT-RM | | No SV |
| NA18484 | *1/*17 | *1/*17 | *61-like/*78 | *1/*17 | GeT-RM | | No SV |
| NA18509 | *2/*17 | *17/*2 | *17/*2 | *2/*17 | GeT-RM | | No SV |
| NA18518 | *17/*29 | *17/*29 | *17/*29 | *17/*29 | GeT-RM | | No SV |
| NA18519 | *1/*29 | *106/*29 | *106/*29 | *29/*106 | GeT-RM | | No SV |
| NA18544 | *10/*41 | *10/*41 | *10/*41 | *10/*41 | GeT-RM | | No SV |
| NA18552 | *1/*14 | *1/*14 | *1/*14 | *1/*14 | GeT-RM | | No SV |
| NA18942 | *2/*2 | *2/*2 | *2/*2 | *2/*2 | GeT-RM | | No SV |
| NA18952 | *2/*2 | *2/*2 | *2/*2 | *2/*2 | GeT-RM | | No SV |
| NA18966 | *1/*2 | *1/*2 | *1/*2 | *1/*2 | GeT-RM | | No SV |
| NA18973 | *1/*21 | *1/*21 | *1/*21 | *2D/*21 | GeT-RM | | No SV |
| NA19003 | *1/*1 | *1/*1 | *1/*1 | *1/*1 | GeT-RM | | No SV |
| NA19007 | *1/*1 | *1/*1 | *1/*1 | *1/*1 | GeT-RM | | No SV |
| NA19095 | *1/*29 | *1/*29 | *1/*29 | *1/*29 | GeT-RM | | No SV |
| NA19122 | *2/*17 | *17/*2 | *17/*2 | *2/*17 | GeT-RM | | No SV |
| NA19143 | *2 (*45)/*10 | *10/*45 | *10/*45 | *2/*10 | GeT-RM | | No SV |
| NA19147 | *17/*29 | *17/*29 | *17/*29 | *17/*29 | GeT-RM | | No SV |
| NA19174 | *4/*40 | *40/*4 | *4/*40 | *4/*40 | GeT-RM | | No SV |
| NA19176 | *1/*2 | *1/*2 | *1/*2 | *1/*2 | GeT-RM | | No SV |
| NA19178 | *1/*1 | *1/*1 | *1/*1 | *1/*1 | GeT-RM | | No SV |
| NA19213 | *1/*1 | *1/*1 | *1/*1 | *1/*1 | GeT-RM | | No SV |
| NA19239 | *15/*17 | *15/*17 | *15/*17 | *2/*15 | GeT-RM | | No SV |
| NA19789 | *1/*1 | *1/*1 | *1/*61 | *1/*1 | GeT-RM | | No SV |
| NA19908 | *1/*46 | *1/*46; | *43/*45 | *1/*46 | GeT-RM | | No SV |
| | | *43/*45 | | | | | |
| NA19917 | *1/*40 | *1/*40 | *1/*40 | *1/*40 | GeT-RM | | No SV |
| NA20296 | *1/*2 | *1/*2 | *1/*2 | *1/*2D | GeT-RM | | No SV |
| NA20509 | *4/*35 | *35/*4 | *35/*4 | *4/*35 | GeT-RM | | No SV |
| NA23275 | *1/*40 | *1/*17 | *1/*40 | *1/*40 | GeT-RM | | No SV |
| | | (updated to | | | | | |
| | | *1/*40) | | | | | |
| NA23348 | *7/*35 | *35/*7 | *35/*7 | *7/*35 | GeT-RM | | No SV |
| HG03882 | *1/*112 | *1/*112 | *1/*1- | *1/*1 | GeT-RM | 1 kGP | No SV |
| | | | like + *61 | | | | |
| HG03780 | *1/*112 | *1/*112 | *1/*1-like | *1/*1 | GeT-RM | 1 kGP | No SV |
| NA19238 | *1/*17 | *1/*17 | *1/*17 | *1/*17 | GeT-RM, | 1 kGP | No SV |
| | | | | | PacBio | | |
| NA20803 | *2/*22 | *2/*22 | *2/*22 | *1/*2 | GeT-RM | 1 kGP | No SV |
| HG04206 | *2/*113 | *113/*2 | *1/*2 | *2/*113 | GeT-RM | 1 kGP | No SV |
| HG01108 | *2/*106 | *106/*2 | *106/*2 | *2/*106 | GeT-RM | 1 kGP | No SV |
| NA20875 | *1/*111 | *1/*111 | *111/*2 | *1/*2 | GeT-RM | 1 kGP | No SV |
| HG01094 | *1/*31 | *1/*31 | *1/*31 | *1/*31 | GeT-RM | 1 kGP | No SV |
| HG01086 | *1/*31 | *1/*31 | *1/*31 | *1/*31 | GeT-RM | 1 kGP | No SV |
| NA07048 | *1/*4 | *1/*4 | *10/*74-like | *1/*4 | GeT-RM | 1 kGP | No SV |
| HG03703 | *1/*99 | *1/*99 | *1/*10 | *1/*99 | GeT-RM | 1 kGP | No SV |
| NA20289 | *6/*11 | *11/*6 | *11/*6 | *6/*11 | GeT-RM | 1 kGP | No SV |
| NA19700 | *4/*29 | *29/*4 | *29/*4 | *4/*29 | GeT-RM | 1 kGP | No SV |
| HG00373 | *2/*2 | *2/*2 | *2/*2 | *2/*2 | GeT-RM | 1 kGP | No SV |
| NA21105 | *3/*111 | *111/*3 | *2/*3-like | *2/*3 | GeT-RM | 1 kGP | No SV |
| NA11881 | *2/*3 | *2/*3 | *2/*3 | *2/*3 | GeT-RM | 1 kGP | No SV |
| NA12815 | *2/*41 | *2/*41 | *2/*41 | *2/*41 | GeT-RM | 1 kGP | No SV |
| HG01680 | *28/*59 | *28/*59 | *28/*59 | *28/*59 | GeT-RM | 1 kGP | No SV |
| HG03643 | *2/*7 | *2/*7 | *2/*7 | *2/*7 | GeT-RM | 1 kGP | No SV |
| HG03781 | *2/*99 | *2/*99 | *10/*2 | *2/*99 | GeT-RM | 1 kGP | No SV |
| HG00111 | *3/*3 | *3/*3 | *3/*3 | *3/*3 | GeT-RM | 1 kGP | No SV |
| NA06989 | *9/*9 | *9/*9 | *9/*9-like | *9/*9 | GeT-RM | 1 kGP | No SV |
| NA19777 | *1/*82 | *1/*82 | *1/*82 | *1/*1 | GeT-RM | 1 kGP | No SV |
| HG02723 | *17/*2 | *17/*2 | *17/*2 | *2/*17 | PacBio | | No SV |
| HG03522 | *1/*1 | *1/*1 | *1/*1 | *1/*1 | PacBio | | No SV |
| HG00450 | *10/*41 | *10/*41 | *10/*41 | *10/*41 | PacBio | | No SV |
| HG03453 | *1/*29 | *1/*29 | *1/*29 | *1/*29 | PacBio | | No SV |
| HG01687 | *1/*6 | *1/*6 | *1/*6 | *1/*6 | PacBio | | No SV |
| HG02984 | *2/*4 | *2/*4 | *2/*4 | *2/*4 | PacBio | | No SV |
| HG01763 | *1/*1 | *1/*1 | *1/*1 | *1/*1 | PacBio | | No SV |
| HG03098 | *2/*29 | *2/*29 | *2/*29 | *2/*29 | PacBio | | No SV |
| HG03041 | *17/*29 | *17/*29 | *17/*29 | *17/*29 | PacBio | | No SV |
| HG02622 | *17/*46 | *17/*46 | *61-like/*78 | *17/*46 | PacBio | | No SV |
| HG01621 | *2/*33 | *2/*33 | *2/*33 | *2/*33 | PacBio | | No SV |
| HG03579 | *1/*2 | *1/*2 | *1/*2 | *1/*2 | PacBio | | No SV |
| HG02975 | *17/*29 | *17/*29 | *17/*29 | *17/*29 | PacBio | | No SV |
| HG03101 | *1/*17 | *1/*17 | *1/*17 | *1/*17 | PacBio | | No SV |
| HG03065 | *106/*29 | *106/*29 | *106/*29 | *29/*106 | PacBio | | No SV |
| HG03486 | *1/*17 | *1/*17 | *1/*17 | *1/*17 | PacBio | | No SV |
| HG03308 | *1/*29 | *1/*29 | *1/*29 | *1/*29 | PacBio | | No SV |
| HG00513 | *10/*10 | *10/*10 | *10/*10 | *10/*10 | PacBio | 1 kGP | No SV |
| HG00143 | *1/*4 | *1/*4 | *1/*4 | *1/*4 | PacBio | 1 kGP | No SV |
| NA20527 | *2/*4 | *2/*4 | *2/*4 | *2/*4 | PacBio | 1 kGP | No SV |
| HG00732 | *41/*9 | *41/*9 | *41/*9 | *9/*41 | PacBio | 1 kGP | No SV |
| HG01119 | *1/*4 | *1/*4 | *1/*4 | *1/*4 | PacBio | 1 kGP | No SV |
| HG00554 | *4/*4 | *4/*4 | *4/*4 | *4/*4 | PacBio | 1 kGP | No SV |
| HG01254 | *2/*41 | *2/*41 | *2/*41 | *2/*41 | PacBio | 1 kGP | No SV |
| HG00186 | *2/*4 | *2/*4 | *2/*4 | *2/*4 | PacBio | 1 kGP | No SV |
| HG00263 | *1/*35 | *1/*35 | *1/*35 | *1/*2 | PacBio | 1 kGP | No SV |
| NA19239 | *15/*17 | *15/*17 | *15/*17 | *1/*15 | PacBio | 1 kGP | No SV |
| NA19437 | *17/*2 | *17/*2 | *17/*2 | *2/*17 | PacBio | 1 kGP | No SV |
| NA19449 | *1/*17 | *1/*17 | *1/*17 | *1/*17 | PacBio | 1 kGP | No SV |
| HG002 | *2/*4 | *2/*4 | *2/*4 | *2/*4 | PacBio | | No SV |
| (NA24385) | | | | | | | |

By comparing against the GeT-RM samples, three samples were found where the calls of all three callers agree but disagree with GeT-RM consensus. Whole genome PacBio sequencing confirmed that the three callers' calls were correct and the GeT-RM consensus should be updated (FIG. 24).

FIG. 24 shows structural variants validated by PacBio CCS reads. PacBio reads supporting deletion (*5), duplications, and fusions (*36, *68 and *13). Plots were generated using sv-viz2 (zotero.org/google-docs/?xAunA6. For deletions and duplications, due to the identical sequence in the REP6/7 regions, the exact positions of the breakpoints within REP6/7 were not available. The breakpoints in A and B are for illustration purposes only. The genotypes of samples in Panel A-E are *2/*5, *17/*2×2, *10/*10+*36, *29/*4+*68 and *1/*2+*13, respectively.

Cyrius initially made four discordant calls from the truth GeT-RM, showing a sensitivity of 97.9%. Included amongst these discrepancies was the sample NA19908 (GeT-RM defined *1/*46), where Cyrius called *1/*46 and *43/*45 as two possible diplotypes. Both of these two star allele combinations result in the same set of variants. Neither read phasing or population frequency analysis could rule out either genotype combination. The genotyping results from various assays that generated the GeT-RM consensus for this sample also showed disagreement between *1/*46 and *43/*45, highlighting the difficulty of these combinations (Table 22). Future sequencing of more samples of either diplotypes could help identify new variants that distinguish the two.

TABLE 22
GeT-RM results for sample NA19908.

| | | | | | iPLEX | |
|---|---|---|---|---|---|---|
| | | | | | CYP2D6 | |
| | | | PharmacoScan | | V1.1 + | |
| | TaqMan + | | custom | iPLEX | custom | Sequencing |
| Consensus | CNVs + | PharmacoScan | v.r6 + | CYP2D6 | panel and | Sanger, NGS |
| genotype | XL-PCR | v.r6 | 20180103 | V1.1 | VeriDose | or SMRT |
| *1/*46 | *1/*45 | *1/*2 | *1/*46, *43/*45 | *1/*46 | n/a | *46 (ASXL- |
| | | | | | | PCR) Sanger; |
| | | | | | | NGS |

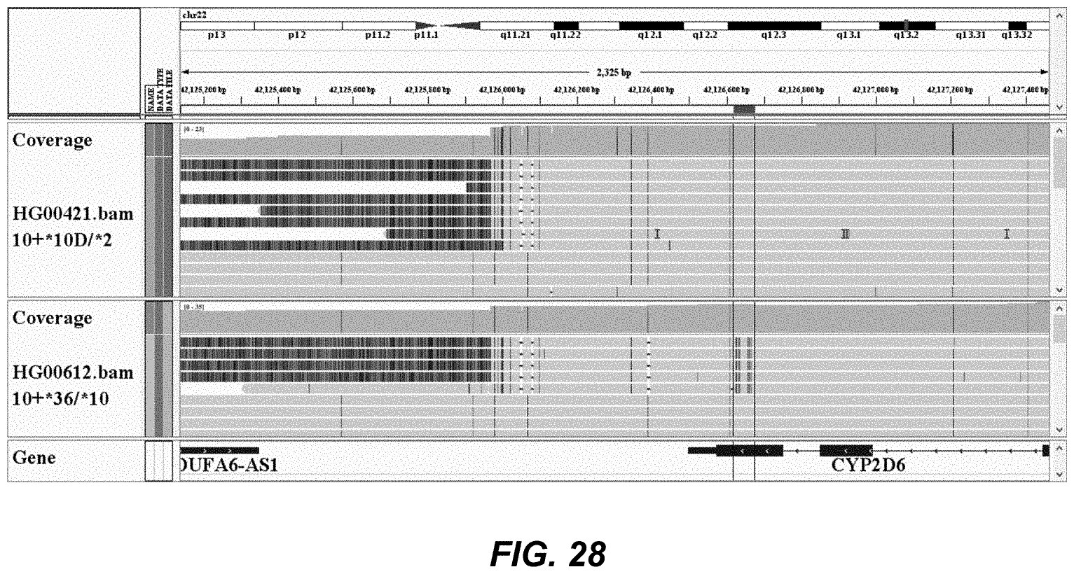

In the remaining three samples where Cyrius was discordant with the truth, the errors made were identified, and Cyrius was improved to call the correct genotypes. First, in NA23275 (*1/*40), the 18 bp insertion defining *40 was originally missed as the insertion-containing reads were often not aligned as having an insertion but as soft-clips. The caller was improved to consider soft-clips when looking for the variant. Second, in HG03225 (*5/*56), CYP2D7-derived reads were aligned to CYP2D6, preventing the *56 defining variant from being called. The caller was improved to be more sensitive to variant reads in this region. Lastly, in HG00421 (*10×2/*2), the fusion was miscalled to be *36, as did the other two callers. Closer examination of this sample with PacBio data showed a different fusion, *10D, with the fusion breakpoint located downstream of Exon 9 (FIG. 28). This fusion has the same function as *10 (decreased function), while *36 has no function due to CYP2D7-derived exon9. The caller was improved to be able to call *10D. While these three samples were treated as miscalls in this Example, the improvements made to Cyrius after seeing these three samples allowed calling 187 of the 188 samples accurately, highlighting how more truth data and more population data can identify limitations that can enable improvements to the caller for subsequent samples.

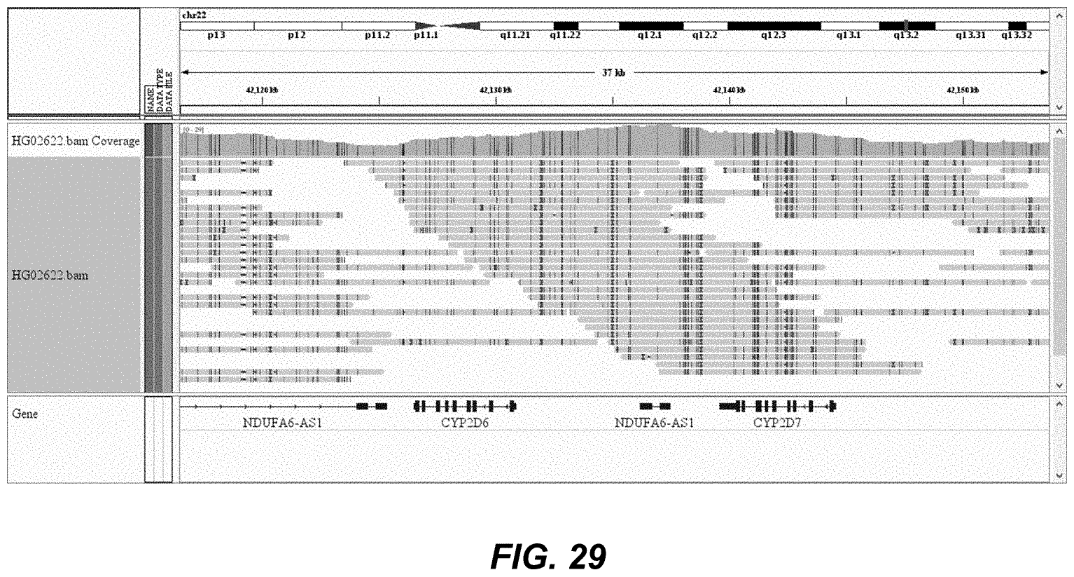

In contrast, both of the other CYP2D6 callers had sensitivity less than 90% when compared against these samples. Aldy had a sensitivity of 88.8%. In particular, it overcalled CYP2D6/CYP2D7 fusions such as *61, *63, *78 and *83 (called in 8 out of 21 discordant samples, Table 21). An Aldy-called fusion can be disproved by PacBio data in FIG. 29. Stargazer had a sensitivity of 85.6% and was most prone to errors when SVs were present. The sensitivity in samples with SVs was only 77.8%, and 16 out of the 27 discordant calls were in samples with structural variants. Notably, it miscalled NA19317 (*5*5) as *2/*2, completely missing the double deletion. Stargazer was not able to genotype the two samples with the *13 fusion (Table 21). In addition, Stargazer showed a high error rate with the *36 fusion (7 wrong calls out of 25 total samples with *36). Particularly, Stargazer miscalled all 5 samples in which there are more than one copy of *36 in one haplotype.

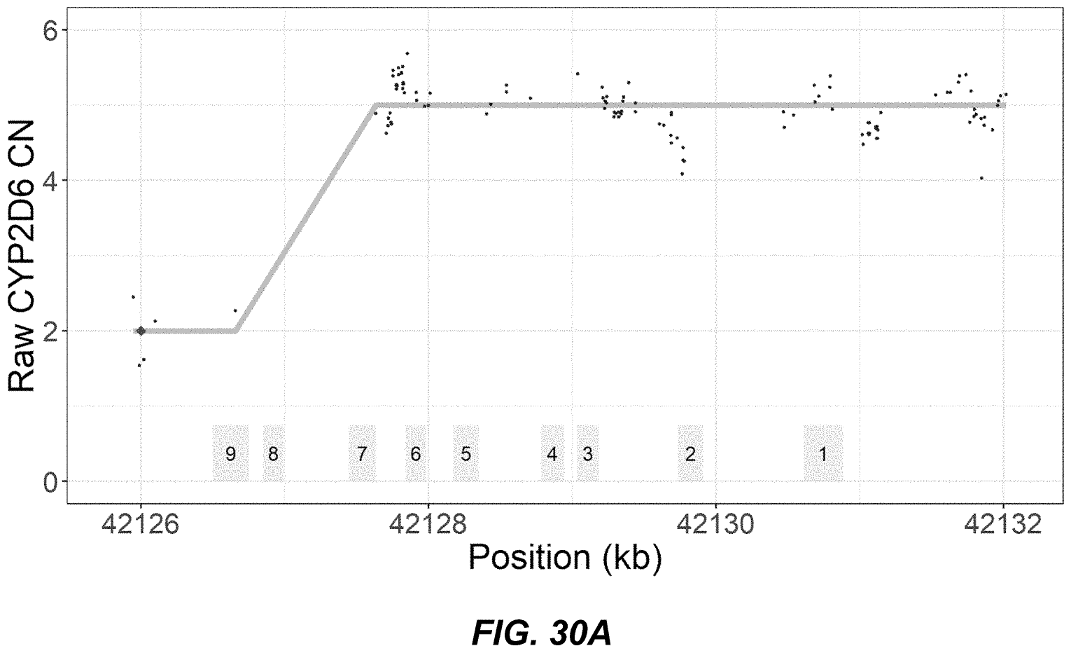

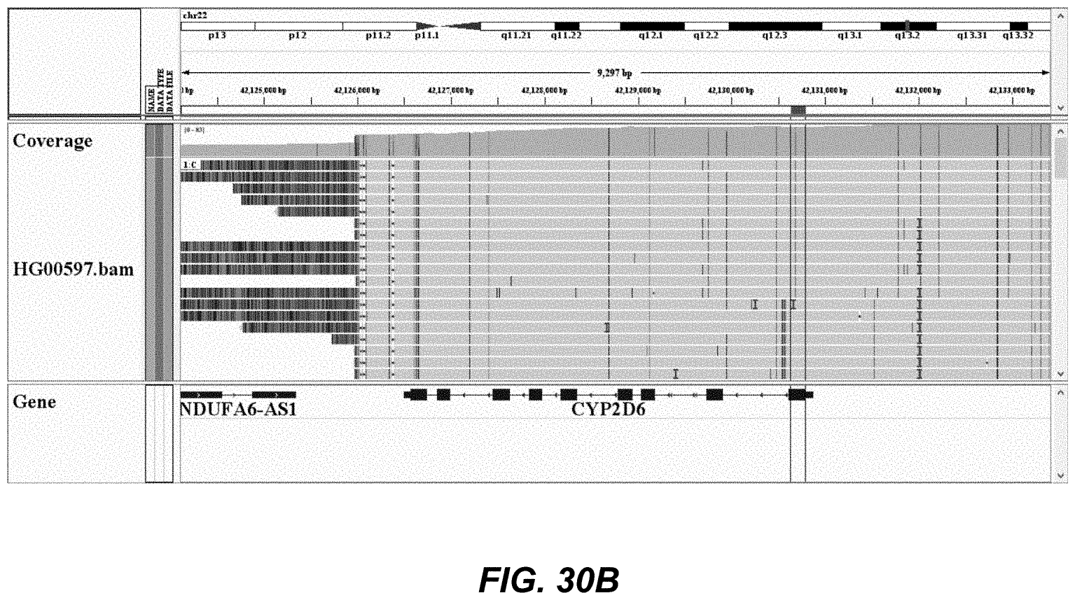

Together, the 188 validation samples used in this example confirmed CYP2D6 calling accuracy of Cyrius in 48 distinct haplotypes (Table 23), including 41 star alleles as well as several common and rare SV structures, such as duplications, *2+*13, *4+*68, *10+*36, *10+*36+*36 and *10+*36+*36+*83 (a novel haplotype that has not been reported before, see FIGS. 30A and 30B). These 41 star alleles that had been tested in the validation data represent 38.7% of the 106 curated star alleles currently listed in PharmVar and 53.4% (31 out of 58) of the ones with known function. They overlap 96.4% of the haplotypes Cyrius called from the 1kGP samples (Table 23, also see next section).

TABLE 23
Haplotypes validated in this example and their frequencies in 1 kGP.

| | | | | | | | Validated | In GeT- | |
|---|---|---|---|---|---|---|---|---|---|
| | Pan- | | Admixed- | East- | | South- | in this | RM full | |
| Haplotype | ethnic | European | American | Asian | African | Asian | example | set | Function |
| *1 | 33.43 | 35.79 | 45.97 | 26.19 | 26.25 | 39.26 | x | x | Normal |
| *2 | 14.86 | 16.2 | 18.44 | 7.74 | 13.24 | 20.45 | x | x | Normal |
| *3 | 0.54 | 1.79 | 0.58 | 0 | 0.23 | 0.2 | x | x | None |
| *4 | 5.83 | 11.83 | 8.79 | 0.2 | 2.34 | 8.08 | x | x | None |
| *5 | 3.49 | 2.39 | 2.02 | 3.47 | 5.82 | 2.56 | x | x | None |
| *6 | 0.5 | 2.09 | 0.29 | 0 | 0.08 | 0.1 | x | x | None |
| *7 | 0.18 | 0 | 0 | 0 | 0 | 0.92 | x | x | None |
| *9 | 0.7 | 2.49 | 1.3 | 0 | 0.08 | 0 | x | x | Decreased |
| *10 | 5.41 | 1.39 | 1.44 | 15.08 | 4.39 | 3.78 | x | x | Decreased |
| *11 | 0.02 | 0 | 0 | 0 | 0.08 | 0 | x | x | None |
| *13 | 0.1 | 0.2 | 0.14 | 0 | 0.08 | 0.1 | x | x | None |
| *14 | 0.18 | 0 | 0 | 0.89 | 0 | 0 | x | x | Decreased |
| *15 | 0.06 | 0 | 0 | 0 | 0.23 | 0 | x | x | None |
| *17 | 5.25 | 0.2 | 0.86 | 0 | 19.29 | 0 | x | x | Decreased |
| *21 | 0.1 | 0 | 0 | 0.5 | 0 | 0 | x | x | None |
| *22 | 0.06 | 0.3 | 0 | 0 | 0 | 0 | x | x | Unknown |
| *28 | 0.12 | 0.5 | 0.14 | 0 | 0 | 0 | x | x | Unknown |
| *29 | 2.64 | 0 | 0.29 | 0 | 9.83 | 0 | x | x | Decreased |
| *31 | 0.12 | 0.2 | 0.58 | 0 | 0 | 0 | x | x | None |
| *33 | 0.18 | 0.6 | 0.29 | 0 | 0 | 0.1 | x | x | Normal |
| *34 | 0.02 | 0 | 0 | 0 | 0.08 | 0 | | | Normal |
| *35 | 1.48 | 4.77 | 2.45 | 0 | 0.23 | 0.61 | x | x | Normal |
| *36 | 0.1 | 0 | 0 | 0.1 | 0.3 | 0 | | | None |
| *39 | 0.08 | 0 | 0.14 | 0 | 0.08 | 0.2 | | x | Normal |
| *40 | 0.24 | 0 | 0 | 0 | 0.91 | 0 | x | x | None |
| *41 | 6.15 | 9.05 | 6.05 | 3.77 | 1.59 | 11.86 | x | x | Decreased |
| *43 | 0.5 | 0.1 | 0 | 0 | 1.06 | 1.02 | x | x | Unknown |
| *45 | 0.88 | 0 | 0.29 | 0 | 3.18 | 0 | x | x | Normal |
| *46 | 0.14 | 0 | 0.14 | 0 | 0.45 | 0 | x | x | Normal |
| *49 | 0.1 | 0 | 0 | 0.5 | 0 | 0 | x | | Decreased |
| *52 | 0.02 | 0 | 0 | 0.1 | 0 | 0 | x | x | Unknown |
| *56 | 0.02 | 0 | 0 | 0 | 0.08 | 0 | x | x | None |
| *59 | 0.06 | 0.2 | 0.14 | 0 | 0 | 0 | x | x | Decreased |
| *71 | 0.12 | 0 | 0 | 0.6 | 0 | 0 | x | x | Unknown |
| *82 | 0.06 | 0 | 0.43 | 0 | 0 | 0 | x | x | Unknown |
| *84 | 0.02 | 0 | 0 | 0 | 0.08 | 0 | | | Decreased |
| *86 | 0.44 | 0 | 0 | 0 | 0 | 2.25 | | | Unknown |
| *99 | 0.04 | 0 | 0 | 0 | 0 | 0.2 | x | x | None |
| *106 | 0.32 | 0 | 0.14 | 0 | 1.13 | 0 | x | x | Unknown |
| *108 | 0.06 | 0.3 | 0 | 0 | 0 | 0 | | x | Unknown |
| *111 | 0.16 | 0 | 0 | 0 | 0 | 0.82 | x | x | Unknown |
| *112 | 0.04 | 0 | 0 | 0 | 0 | 0.2 | x | x | Unknown |
| *113 | 0.16 | 0 | 0 | 0 | 0 | 0.82 | x | x | Unknown |
| *1 × 2 | 0.5 | 0.5 | 1.15 | 0.1 | 0.45 | 0.51 | x | x | Increased |
| *1 × 3 | 0.02 | 0 | 0 | 0 | 0.08 | 0 | | | Increased |
| *2 × 2 | 1.14 | 1.49 | 0.58 | 0.6 | 2.12 | 0.41 | x | x | Increased |
| *2 × 3 | 0.04 | 0.1 | 0 | 0 | 0.08 | 0 | | | Increased |
| *4 × 2 | 0.84 | 0.3 | 0.14 | 0 | 2.87 | 0 | x | x | None |
| *4 × 3 | 0.04 | 0 | 0 | 0 | 0.15 | 0 | | | None |
| *9 × 2 | 0.02 | 0.1 | 0 | 0 | 0 | 0 | | | Normal |
| *10 × 2 | 0.06 | 0 | 0 | 0.3 | 0 | 0 | x | x | Decreased |
| *17 × 2 | 0.02 | 0 | 0 | 0 | 0.08 | 0 | | x | Normal |
| *29 × 2 | 0.1 | 0 | 0 | 0 | 0.38 | 0 | | | Normal |
| *35 × 2 | 0.02 | 0 | 0.14 | 0 | 0 | 0 | | | Increased |
| *43 × 2 | 0.04 | 0 | 0.14 | 0 | 0.08 | 0 | | | Unknown |
| *45 × 3 | 0.02 | 0 | 0 | 0 | 0.08 | 0 | | | Increased |
| *10 + *36 | 7.23 | 0 | 0.14 | 34.62 | 0.08 | 1.12 | x | x | Decreased |
| *4 + *68 | 1.94 | 5.57 | 2.45 | 0 | 0.23 | 2.15 | x | x | None |
| *4 + *68 + | 0.08 | 0.1 | 0.43 | 0 | 0 | 0 | | | None |
| *68 | | | | | | | | | |
| *10 + *36 + | 0.32 | 0 | 0 | 1.59 | 0 | 0 | x | x | Decreased |
| *36 | | | | | | | | | |
| *10 + *36 + | 0.02 | 0 | 0 | 0.1 | 0 | 0 | x | x | Decreased |
| *36 + *36 | | | | | | | | | |
| *2 + *13 | 0.06 | 0.2 | 0.14 | 0 | 0 | 0 | x | x | Normal |
| *4 + *4N | 0.14 | 0.7 | 0 | 0 | 0 | 0 | | x | None |
| *1 + *90 | 0.02 | 0 | 0 | 0.1 | 0 | 0 | x | x | Unknown |
| *10 + *36 + | 0.02 | 0 | 0 | 0.1 | 0 | 0 | x | | Decreased |
| *36 + *83 | | | | | | | | | |
| Unknown | 2.36 | 0.6 | 3.75 | 3.37 | 2.27 | 2.25 | | | |
| % haplotypes | 96.4 | 98.1 | 95.4 | 96.5 | 96.3 | 95.3 | | | |
| overlapping the | | | | | | | | | |
| validation set | | | | | | | | | |

CYP2D6 Haplotype Frequencies Across Five Ethnic Populations

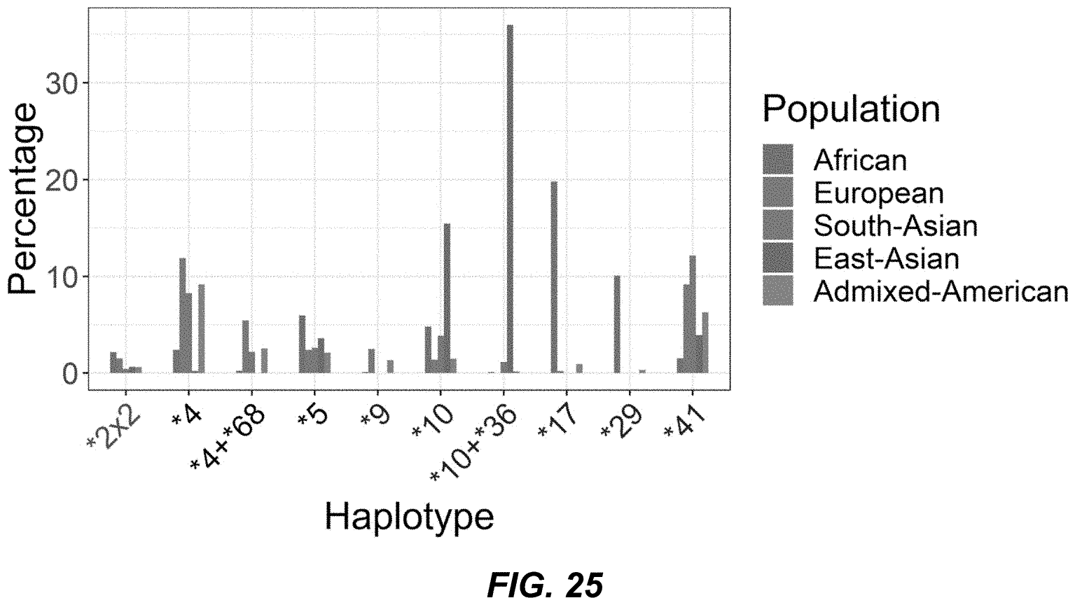

Given the high accuracy demonstrated in the previous section, Cyrius was used beyond the validation samples to study CYP2D6 in the global population. The haplotype distribution by population (Europeans, Africans, East Asians, South Asians and admixed Americans consisting of Colombians, Mexican-Americans, Peruvians and Puerto Ricans) in 2504 1kGP samples was analyzed (FIG. 25, Table 23). Cyrius made definitive diplotype calls in 2445 (97.6%) out of 2504 samples calling 46 distinct star alleles, of which 41 star alleles overlapped those that had been included in the validation data. These 41 previously validated star allele calls represent 96.5% of all the star alleles called in the 1kGP samples (Table 23).

FIG. 25 is a non-limiting exemplary plot showing CYP2D6 allele frequencies across five ethnic populations with ten most common haplotypes with altered CYP2D6 function. One haplotype (*2×2) has increased function, two haplotypes (*4 and *4+*68) have no function, and the remaining haplotypes have decreased function.

In the 59 samples where Cyrius did not make a definitive diplotype call, 10 samples had a nondefinitive SV call, 30 samples had variant calls that did not match any of the known star alleles, four samples had the same ambiguity between *1/*46 and *43/*45 as described in the validation sample NA19908 above, and 15 samples had definitive star allele calls that Cyrius was not able to unambiguously phase into diplotypes.

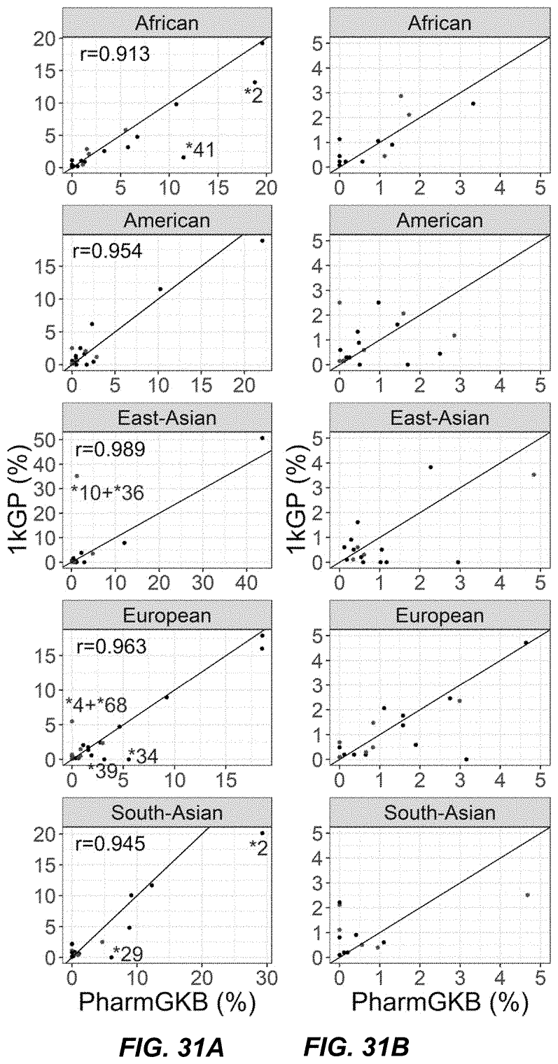

The haplotype frequencies agree with pharmGKB in most cases (FIGS. 31A and 31B, Table 24). For example, Africans have a high frequency of *17 (˜20%) and *29 (˜10%), South-Asians have a high frequency of *41 (12%), Europeans have a high frequency of *4 (18-20%, including *4+*68), and East Asians have a high frequency of *10 (40%-50%, including *10+*36). With the improved sensitivity for structural variants by Cyrius, providing a more comprehensive picture on the frequencies of structural variants across populations is possible. Among those, the fusion-containing haplotype *10+*36 is very common in East Asians (>30%, compared to 1-2% reported in pharmGKB, FIGS. 31A and 31B), and another fusion-containing haplotype *4+*68 is also quite common in Europeans (>5%, data not available in pharmGKB, FIGS. 31A and 31B). Together, the frequencies of haplotypes involving SVs were estimated to be 32.2%, 5.57%, 1.47%, 1.34% and 0.45% more than reported in PharmGKB in East-Asians, Europeans, Americans, Africans and South-Asians, respectively (total reported frequencies in PharmGKB are 7.48%, 5.33%, 5.17%, 9.9% and 6.19% respectively).

There are a few other haplotypes for which a lower frequency than PharmGKB was reported (FIGS. 31A and 31B), highlighting the difficulty of merging data from multiple studies using different technologies. These include *2 in Africans and South-Asians. Since *2 is a default assignment if some other star alleles are not tested, its frequency could be overestimated in PharmGKB. A lower frequency for *41 in Africans was determined. According to PharmGKB, *41 has not consistently been determined by its defining SNP across studies, leading to an overestimation of the*41 frequency, especially in those of African ancestry. The much higher frequency of *29 in South-Asians in PharmGKB (6% vs. 0% estimated in this example) was caused by an error in PharmGKB: 0.2% in Sistonen et al. (CYP2D6 worldwide genetic variation shows high frequency of altered activity variants and no continental structure. Pharmacogenet Genomics. 2007; 17(2):93-101. doi:10.1097/01.fpc.0000239974.69464.f2) was included erroneously in PharmGKB as 20%. In Europeans, a much lower frequency for *34 and *39 were estimated. *34 and *39 are each defined by one of the two variants that define *2, so both of these two variants should have been tested in any study that profiles CYP2D6. *34 and *39 are reported at >1% by only 3 out of 91 studies on Europeans in PharmGKB, among which Wesmiller et al. (The Association of CYP2D6 Genotype and Postoperative Nausea and Vomiting in Orthopedic Trauma Patients. Biol Res Nurs. 2013; 15(4):382-389. doi:10.1177/1099800412449181) which reported *39 only and had a limited sample size (N=112), Kapedanovska Nestorovska (Distribution of the most Common Genetic Variants Associated with a Variable Drug Response in the Population of the Republic of Macedonia. Balk J Med Genet BJMG. 2014; 17(2):5-14. doi:10.2478/bjmg-2014-0069) reported both *34 and *39 and was for a specific country Macedonia and also had a small sample size (N=184), and Del Tredici et al. (Frequency of CYP2D6 Alleles Including Structural Variants in the United States. Front Pharmacol. 2018; 9. doi:10.3389/fphar.2018.00305), did not report *34 or *39 but PharmGKB may have erroneously took the frequency reported for *35 as that for *34.

Analysis on CYP2D6/CYP2D7 Differentiating Bases

A total of 208 single nucleotide differences between CYP2D6/7 were pulled out from the reference genome. In 1kGP samples where a total CYP2D6+CYP2D7 CN of 4, i.e. no structural variations were called, the percentage of samples where the CN of CYP2D6 base called as 2 across the 208 sites was queried (FIG. 26). Many sites showed a low percentage of samples with two copies of CYP2D6 base, suggesting that the CYP2D6/CYP2D7 base difference is not fixed in the population, so the base differences cannot be used to differentiate between the two genes. Relying on read alignments to these sites would result in a large amount of noise when differentiating the two genes. A total of 118 highly stable sites with >98% of samples showing two copies of CYP2D6 bases for CYP2D6/CYP2D7 differentiation were selected, which allowed gaining the cleanest signal for calling SVs.

### Additional Figures and Tables

FIG. 26 shows that CYP2D6/CYP2D7 base difference sites have high variability in the population. The y axis shows the frequency of samples where the CN of the CYP2D6 base is called at 2 out of all samples that have a total CYP2D6+CYP2D7 CN of 4. The x axis shows genome coordinates in hg38. CYP2D6 exons are drawn as gray boxes above the plot. The black horizontal line denotes the 98% cutoff.

FIG. 27 shows raw CYP2D6 CNs across CYP2D6/7 differentiating sites in examples with SVs. Raw CYP2D6 CN was calculated as the total CYP2D6+CYP2D7 CN multiplied by the ratio of CYP2D6 supporting reads out of CYP2D6 and CYP2D7 supporting reads. The large diamond denotes the copy number of genes that are CYP2D6-derived at the end of the gene (can be complete CYP2D6 or fusion gene ending in CYP2D6), calculated as the total CYP2D6+CYP2D7 CN minus the CN of the CYP2D7 spacer region (see FIG. 23). To detect SVs, a CYP2D6 CN was called at each site and a change in CYP2D6 CN within the gene indicated the presence of SV. For example, in HG01161, the CYP2D6 CN changed from 2 to 1 between Exon 7 and Exon 9, indicating a CYP2D7-CYP2D6 hybrid gene. In HG00553, the CYP2D6 CN changed from 2 to 3 between Exon 1 and Exon 2, indicating a CYP2D6-CYP2D7 hybrid gene.

FIG. 28 shows that PacBio data confirms *10D fusion in HG00421. A sample with *36 (HG00612) is shown in comparison. PacBio reads that contain the fusions are those with shaded bases that represent soft-clips made by the aligner and were derived from CYP2D7 part of the fusion. The fusion breakpoints are close to each other but the breakpoint for *36 is upstream of the base differences in exon 9 (those inside the black box), while the breakpoint for *10D is downstream, leaving the CYP2D6 gene intact.

FIG. 29 shows that PacBio data had a false *61 (CYP2D6/CYP2D7 hybrid) call made by Aldy in HG02622. Expected genotype was *17/*45 but Aldy called *61-like/*78 (both *61 and *78 are star alleles with SVs). PacBio data showed that there was no structural variant in the region (each read aligns completely, with no soft-clips indicating unaligned parts).

FIGS. 30A and 30B show novel *10+*36+*36+*83 haplotype in HG00597. FIG. 30A. Depth plot as in FIG. 27, showing that HG00597 had three copies of *36-like fusions, all of which had a breakpoint in the homology region between Exon 7 and Exon 9. FIG. 30B. IGV screenshot of the PacBio data, showing all the fusion-containing reads, i.e. those that aligned with a soft-clip. One copy of the fusion gene did not have g.42130692G>A, the SNP that was in *36 but not in *83, as shown in the region flanked by two black vertical lines. This copy was *83, and unlike what was reported in PharmVar, this was a fusion gene with REP7 not REP6, otherwise the copy number of the region downstream of exon 9 would be 3 instead of 2 in FIG. 30A.

FIGS. 31A and 31B compare between 1kGP and pharmGKB frequencies. Each dot represents a haplotype with a frequency >=0.5% in either 1kGP or pharmGKB. SV-related haplotypes are marked, including the two haplotypes with the largest deviation (*10+*36 in East-Asians and *4+*68 in Europeans). Other haplotypes with deviated values are annotated (*2, *41, *34, *39, *2, and *29). A diagonal line is drawn for each panel. Correlation coefficients are listed for each population (*10+*36 is excluded in East-Asians and *4+*68 is excluded in Europeans for calculation). FIG. 31B shows values in the low value range (<5%).

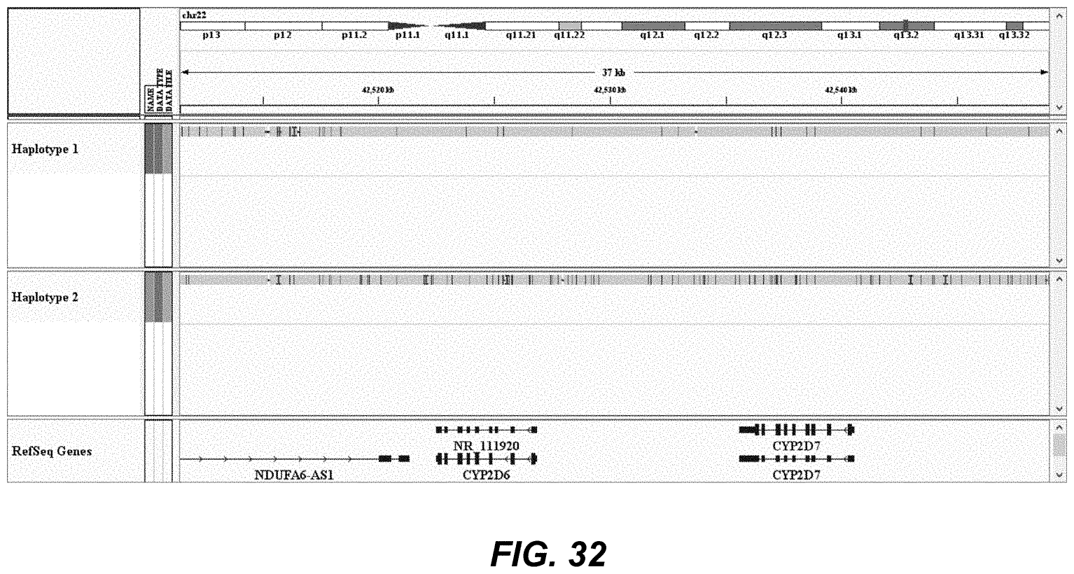

FIG. 32 is anon-limiting exemplary IGV snapshot showing de novo assembly of PacBio reads in HG00733 did not include the *68 fusion.

TABLE 24
Comparison of Cyrius-called haplotype
frequencies and pharmGKB frequencies

| Haplotype | Ethnicity | pharmGKB | 1 kGP | Function |
|---|---|---|---|---|
| *1 | African | 9.53 | 26.786 | Normal function |
| *2 | African | 18.81 | 13.509 | Normal function |
| *3 | African | 0.15 | 0.233 | No function |
| *4 | African | 3.33 | 2.407 | No function |
| *6 | African | 0 | 0.078 | No function |
| *9 | African | 0 | 0.078 | Decreased function |
| *10 | African | 6.71 | 4.814 | Decreased function |
| *15 | African | 0.57 | 0.233 | No function |
| *17 | African | 19.58 | 19.798 | Decreased function |
| *29 | African | 10.73 | 10.093 | Decreased function |
| *35 | African | 0 | 0.233 | Normal function |
| *39 | African | 0 | 0.078 | Normal function |
| *40 | African | 1.31 | 0.932 | No function |
| *41 | African | 11.47 | 1.553 | Decreased function |
| *43 | African | 0.96 | 1.087 | Uncertain function |
| *45 | African | 5.77 | 3.261 | Normal function |
| *46 | African | 0 | 0.466 | Normal function |
| *1 × 2 | African | 1.12 | 0.466 | Increased function |
| *2 × 2 | African | 1.73 | 2.174 | Increased function |
| *4 × 2 | African | 1.53 | 2.95 | No function |
| *5 | African | 5.52 | 5.978 | No function |
| *106 | African | 0 | 1.165 | Uncertain function |
| *1 | American | 51.05 | 47.598 | Normal function |
| *2 | American | 22.09 | 19.219 | Normal function |
| *3 | American | 0.02 | 0.601 | No function |
| *4 | American | 10.25 | 9.159 | No function |
| *6 | American | 0.25 | 0.3 | No function |
| *7 | American | 0.5 | 0 | No function |
| *9 | American | 0.45 | 1.351 | Decreased function |
| *10 | American | 1.44 | 1.502 | Decreased function |
| *12 | American | 1.7 | 0 | No function |
| *17 | American | 0.48 | 0.901 | Decreased function |
| *28 | American | 0.09 | 0.15 | Uncertain function |
| *29 | American | 0.19 | 0.3 | Decreased function |
| *33 | American | 0.17 | 0.3 | Normal function |
| *35 | American | 0.97 | 2.553 | Normal function |
| *41 | American | 2.33 | 6.306 | Decreased function |
| *82 | American | 2.5 | 0.45 | Unknown function |
| *1 × 2 | American | 2.86 | 1.201 | Increased function |
| *2 × 2 | American | 0.61 | 0.601 | Increased function |
| *4 × 2 | American | 0.11 | 0.15 | No function |
| *35 × 2 | American | 0 | 0.15 | Increased function |
| *5 | American | 1.59 | 2.102 | No function |
| *4 + *68 | American | 0 | 2.553 | No function |
| *1 | East-Asian | 24.74 | 27.216 | Normal function |
| *2 | East-Asian | 12.09 | 8.041 | Normal function |
| *4 | East-Asian | 0.54 | 0.206 | No function |
| *10 | East-Asian | 43.56 | 15.464 | Decreased function |
| *14 | East-Asian | 0.29 | 0.928 | Decreased function |
| *21 | East-Asian | 0.35 | 0.515 | No function |
| *34 | East-Asian | 1.02 | 0 | Normal function |
| *39 | East-Asian | 0.59 | 0 | Normal function |
| *41 | East-Asian | 2.27 | 3.918 | Decreased function |
| *49 | East-Asian | 1.05 | 0.515 | Decreased function |
| *52 | East-Asian | 0.18 | 0.103 | Uncertain function |
| *65 | East-Asian | 2.95 | 0 | Uncertain function |
| *69 | East-Asian | 1.17 | 0 | No function |
| *71 | East-Asian | 0.12 | 0.619 | Uncertain function |
| *1 × 2 | East-Asian | 0.34 | 0.103 | Increased function |
| *2 × 2 | East-Asian | 0.45 | 0.619 | Increased function |
| *10 × 2 | East-Asian | 0.61 | 0.309 | Decreased function |
| *5 | East-Asian | 4.84 | 3.608 | No function |
| *10 + *36 | East-Asian | 1.24 | 35.979 | Decreased function |
| *10 + *36 + | East-Asian | 0.45 | 1.649 | Decreased function |
| *36 | | | | |
| *1 | European | 23.79 | 36.593 | Normal function |
| *2 | European | 18.52 | 16.23 | Normal function |
| *3 | European | 1.58 | 1.714 | No function |
| *4 | European | 18.56 | 11.895 | No function |
| *6 | European | 1.11 | 1.915 | No function |
| *9 | European | 2.75 | 2.52 | Decreased function |
| *10 | European | 1.58 | 1.411 | Decreased function |
| *17 | European | 0.36 | 0.202 | Decreased function |
| *28 | European | 0 | 0.504 | Uncertain function |
| *31 | European | 0.12 | 0.202 | No function |
| *33 | European | 1.9 | 0.605 | Normal function |
| *34 | European | 5.54 | 0 | Normal function |
| *35 | European | 4.64 | 4.839 | Normal function |
| *39 | European | 3.16 | 0 | Normal function |
| *41 | European | 9.23 | 9.173 | Decreased function |
| *43 | European | 0 | 0.101 | Uncertain function |
| *59 | European | 0.65 | 0.202 | Decreased function |
| *1 × 2 | European | 0.83 | 0.504 | Increased function |
| *2 × 2 | European | 0.84 | 1.512 | Increased function |
| *4 × 2 | European | 0.66 | 0.302 | No function |
| *9 × 2 | European | 0.01 | 0.101 | Normal function |
| *5 | European | 2.99 | 2.419 | No function |
| *4 + *68 | European | 0 | 5.444 | No function |
| *4 + *4N | European | 0 | 0.706 | No function |
| *1 | South-Asian | 24.9 | 40.147 | Normal function |
| *2 | South-Asian | 29.3 | 20.964 | Normal function |
| *3 | South-Asian | 0.11 | 0.21 | No function |
| *4 | South-Asian | 9.13 | 8.281 | No function |
| *6 | South-Asian | 0 | 0.105 | No function |
| *7 | South-Asian | 0.41 | 0.943 | No function |
| *10 | South-Asian | 8.84 | 3.878 | Decreased function |
| *29 | South-Asian | 6.08 | 0 | Decreased function |
| *35 | South-Asian | 1.1 | 0.629 | Normal function |
| *39 | South-Asian | 0.2 | 0.21 | Normal function |
| *41 | South-Asian | 12.29 | 12.159 | Decreased function |
| *1 × 2 | South-Asian | 0.56 | 0.419 | Increased function |
| *2 × 2 | South-Asian | 0.95 | 0.419 | Increased function |
| *5 | South-Asian | 4.68 | 2.621 | No function |
| *10 + *36 | South-Asian | 0 | 1.153 | Decreased function |
| *4 + *68 | South-Asian | 0 | 2.201 | No function |
| *86 | South-Asian | 0 | 2.306 | Unknown function |
| *111 | South-Asian | 0 | 0.839 | Unknown function |
| *113 | South-Asian | 0 | 0.839 | Unknown function |

### Discussion

This examples describes Cyrius, a method that can accurately diplotype the difficult CYP2D6 region. A unique feature of this example is that long read data was sued to validate both the haplotypes and the SVs. Long reads provide a unique opportunity to confirm the breakpoint regions for common SVs (CYP2D6 deletions and duplications, as well as CYP2D6/7 fusion genes) and confirm the phasing of the CYP2D6 gene. Using 188 samples, including 50 with long read validation data, as an orthogonal validation dataset, Cyrius was shown to outperforms other CYP2D6 genotypers achieving 97.9% accuracy versus 88.8% for Aldy and 85.6% for Stargazer. In particular, compared to these existing CYP2D6 callers, Cyrius allowed for the possibility that reads may be misaligned in the regions where CYP2D6/7 have high-similarity. Ambiguous read alignments in these regions can lead to incorrect copy number estimation and errors in small variant calling. By accounting for possible mis-aligned reads and selecting a set of reliable CYP2D6/7 differentiating sites, Cyrius is able to do a much better job identifying star alleles with SVs, achieving 97.2% accuracy compared to 88.9% for Aldy and 77.8% for Stargazer.

Across these 188 validation samples, a total of 41 different star alleles, representing (38.7%) of all star alleles listed in PharmGKB including 53.4% of the ones with a known functional status, were validated. Even though the validation set included only 38.7% of the total known star alleles, based on analysis of the 1kGP samples in this example, these were estimated to represent 96.5% of the star alleles in the pangenomic population. In general, the allele frequencies calculated for the 2504 1kGP samples from five ethnic populations agreed with previous studies for simple star alleles. Conversely, for some of the star alleles that were defined by the presence of SVs, quite different frequencies were identified, likely because many of the SV-impacted star alleles are difficult to resolve with conventional assays. This highlights the inherent errors of merging results from studies that used a variety of different CYP2D6 assays, some of which may be designed to just call a subset of star alleles. For example, of the 5 assays used to generate the GeT-RM consensus genotypes, the individual accuracy ranged from 47.1% to 75.2% when compared against the consensus (Table 25). A single method that is able to resolve all of the known star alleles from a single assay is a better choice to build a population-level database.

TABLE 25
Accuracy of individual GeT-RM assays.

| | | | | | iPLEX |
|---|---|---|---|---|---|
| | | | | | CYP2D6 |
| | TaqMan + | | PharmacoScan | | V1.1 + |
| | CNVs + | PharmacoScan | custom | iPLEX | custom panel and |
| | XL-PCR | v.r6 | v.r6 + 20180103 | CYP2D6 V1.1 | VeriDose |
| Samples not | 60.9% | 78.2% | 78.2% | 100.0% | 29.1% |
| listed as n/a | | | | | |
| Accuracy | 75.2% | 47.1% | 60.7% | 59.8% | 69.2% |

Furthermore, Cyrius was used to analyze 2504 1kGP samples from five ethnic populations to determine the star allele frequencies. The allele frequencies calculated agree with previous studies for simple star alleles, and Cyrius greatly improved the allele frequency estimates for star alleles involving structural variants, which can be difficult to detect by conventional methods.

Some existing methods rely on accurate alignment of reads to distinguish CYP2D6 and CYP2D7, which can be error prone due to a few high sequence similarity regions between the two genes, particularly intron1-exon2 and exon7-exon9 regions. Ambiguous alignments can lead to noise in depth profiles, resulting in false CNV calls. Additionally, erroneous read alignments can lead to false positive or negative variant calls. In contrast, Cyrius first called the total CN of CYP2D6+CYP2D7 by counting all reads that align to either gene, and a total CN not equal to 4 clearly suggested the existence of SV. To determine the exact position of the SV, not all differences based on the reference genome were used. Many CYP2D6/CYP2D7 base differences are not fixed, so not all of these positions can be used to reliably distinguish CYP2D6 from CYP2D7 (FIG. 26). Cyrius used 118 CYP2D6/CYP2D7 differentiating positions selected to determine the exact position of the SV. By calling the total CN first and then differentiating them using a subset of good differentiating bases, Cyrius was able to achieve more accurate SV calls. For small variant calling, Cyrius overcome the dependency on unambiguous alignments by looking for variant reads both at the CYP2D6 position and the corresponding position in CYP2D7, thus deriving the most accurate small variant calls.

In his example, long read data was used to validate both the haplotypes and the SV calls. PacBio data in this example provides a clear picture of the CYP2D6-CYP2D7 region with high quality long reads (10-20 kb). Particularly, PacBio data helps resolve the breakpoint regions for common structural variants (CYP2D6 deletions and duplications, as well as CYP2D6-CYP2D7 fusion genes). Even with PacBio reads, genotyping CYP2D6 may not be straightforward and can require a targeted analysis, especially for structural variants involving duplications (CYP2D6 duplications and CYP2D6-CYP2D7 duplication fusions), where the duplicated region is >10 kb. For example, a de novo assembly approach failed to capture the *68 fusion in sample HG00733 (FIGS. 31A and 31B). Furthermore, PacBio reads are not long enough to cover more than one copy of the duplicated sequence, and PacBio reads are too long for CN calling based on read counting (for short reads), so estimating the copy number is difficult. Whole genome sequencing with short reads provides the most accurate solution to genotyping CYP2D6.

In the analysis of the 1kGP samples, Cyrius was able to call a definitive genotype in greater than 97.6% of the samples. Cyrius can solve the remaining 2.4% of the samples in some embodiments. For example, in samples where multiple haplotype configurations are possible, taking a probabilistic approach to derive the most likely genotype given the observed variants can be useful. In addition, continuing to sequence and test more samples will help confirm the ability to genotype rare star alleles and will also identify new variants that can be used to distinguish ambiguous diplotypes. This process was demonstrated in this example where improvements were made to better call three star alleles that were initially mis-called in the 188 validation samples. The improvements were beneficial to population-level genotyping as the three star alleles are found in almost 1% (23 of 2504) of the 1kGP samples.

As new star alleles are identified, the new star alleles can be added into the Cyrius database. One consideration with adding new star alleles that are defined by new variants is that these variants are unlikely to be considered in the previous star allele definitions. As a result, there could exist novel combinations of new and existing variants that could not match any of the known combinations, leading to no-calls. For example, Cyrius includes an option to genotype against 25 new star alleles added in PharmVar v4 (not included in GeT-RM, Aldy or Stargazer). However, five (*119, *122, *135, *136, *139) of the 25 new star alleles have new variants that, when included, led to no-calls in samples that could be called before, suggesting that there exist common novel star alleles with a variant combination not captured in PharmVar. As a result, these five star alleles were removed together with two others (*127, with a gene conversion variant in homology region, and *131, with a variant in a noisy site), keeping the remaining 18. Novel star alleles may be possible as new variants/star alleles are identified. Public WGS datasets like the 2504 1kGP samples analyzed here can be an important component of integrating new variants into the star allele definitions because this data will allow variants to be rapidly assessed across many samples with diverse genotypes.

WGS provides a valuable opportunity to profile all genetic variations of the entire genome but many of regions/variants that are clinically important are beyond the ability of most secondary analysis pipelines. CYP2D6 is among the difficult regions in the genome that are both clinically important and also requires targeted bioinformatics solutions in addition to normal WGS pipelines. Such targeted methods have already been applied successfully to some difficult regions, such as SMN1 gene responsible for spinal muscular atrophy as illustrated in Example 1. The more targeted methods like Cyrius can accelerate pharmacogenomics enable personalized medicine.

### ADDITIONAL CONSIDERATIONS

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

One skilled in the art will appreciate that, for this and other processes and methods disclosed herein, the functions performed in the processes and methods can be implemented in differing order. Furthermore, the outlined steps and operations are only provided as examples, and some of the steps and operations can be optional, combined into fewer steps and operations, or expanded into additional steps and operations without detracting from the essence of the disclosed embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Accordingly, phrases such as “a device configured to” are intended to include one or more recited devices. Such one or more recited devices can also be collectively configured to carry out the stated recitations. For example, “a processor configured to carry out recitations A, B and C can include a first processor configured to carry out recitation A and working in conjunction with a second processor configured to carry out recitations B and C. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

It will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

It is to be understood that not necessarily all objects or advantages may be achieved in accordance with any particular embodiment described herein. Thus, for example, those skilled in the art will recognize that certain embodiments may be configured to operate in a manner that achieves or optimizes one advantage or group of advantages as taught herein without necessarily achieving other objects or advantages as may be taught or suggested herein.

All of the processes described herein may be embodied in, and fully automated via, software code modules executed by a computing system that includes one or more computers or processors. The code modules may be stored in any type of non-transitory computer-readable medium or other computer storage device. Some or all the methods may be embodied in specialized computer hardware.

Many other variations than those described herein will be apparent from this disclosure. For example, depending on the embodiment, certain acts, events, or functions of any of the algorithms described herein can be performed in a different sequence, can be added, merged, or left out altogether (for example, not all described acts or events are necessary for the practice of the algorithms). Moreover, in certain embodiments, acts or events can be performed concurrently, for example through multi-threaded processing, interrupt processing, or multiple processors or processor cores or on other parallel architectures, rather than sequentially. In addition, different tasks or processes can be performed by different machines and/or computing systems that can function together.

The various illustrative logical blocks and modules described in connection with the embodiments disclosed herein can be implemented or performed by a machine, such as a processing unit or processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor can be a microprocessor, but in the alternative, the processor can be a controller, microcontroller, or state machine, combinations of the same, or the like. A processor can include electrical circuitry configured to process computer-executable instructions. In another embodiment, a processor includes an FPGA or other programmable device that performs logic operations without processing computer-executable instructions. A processor can also be implemented as a combination of computing devices, for example a combination of a DSP and a microprocessor, a plurality of microprocessors, one or more microprocessors in conjunction with a DSP core, or any other such configuration. Although described herein primarily with respect to digital technology, a processor may also include primarily analog components. For example, some or all of the signal processing algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

Any process descriptions, elements or blocks in the flow diagrams described herein and/or depicted in the attached figures should be understood as potentially representing modules, segments, or portions of code which include one or more executable instructions for implementing specific logical functions or elements in the process. Alternate implementations are included within the scope of the embodiments described herein in which elements or functions may be deleted, executed out of order from that shown, or discussed, including substantially concurrently or in reverse order, depending on the functionality involved as would be understood by those skilled in the art.

It should be emphasized that many variations and modifications may be made to the above-described embodiments, the elements of which are to be understood as being among other acceptable examples. All such modifications and variations are intended to be included herein within the scope of this disclosure and protected by the following claims.

1. A method for determining a copy number of survival of motor neuron 1 (SMN1) gene comprising:
under control of a hardware processor:
receiving sequence data comprising a plurality of sequence reads obtained from a sample of a subject aligned to survival of motor neuron 1 (SMN1) gene or survival of motor neuron 2 (SMN2) gene;
determining (i) a first number of sequence reads of the plurality of sequence reads aligned to a first SMN1 or SMN2 region comprising at least one of exon 1 to exon 6 of the SMN1 gene or the SMN2 gene, respectively, and (ii) a second number of sequence reads of the plurality of sequence reads aligned to a second SMN1 or SMN2 region comprising at least one of exon 7 and exon 8 of the SMN1 gene or the SMN2 gene, respectively;
determining (i) a first normalized number of the sequence reads aligned to the first SMN1 or SMN2 region and (ii) a second normalized number of the sequence reads aligned to the second SMN1 or SMN2 region using (i) a length of the first SMN or SMN2 region and (ii) a length of the second SMN or SMN2 region, respectively;
determining (i) a copy number of total survival of motor neuron (SMN) genes, each being an intact SMN1 gene, an intact SMN2 gene, a truncated SMN1 gene, or a truncated SMN2 gene, and (ii) a copy number of any intact SMN genes, each being the intact SMN1 gene or the intact SMN2 gene, using a Gaussian mixture model comprising a plurality of Gaussians each representing a different integer copy number, given (i) the first normalized number of the sequence reads aligned to the first SMN or SMN2 region and (ii) the second normalized number of the sequence reads aligned to the second SMN or SMN2 region, respectively;
for one of a plurality of SMN gene-specific bases associated with the intact SMN1 gene, determining a most likely combination, of a plurality of possible combinations each comprising a possible copy number of the SMN1 gene and a possible copy number of the SMN2 gene summed to the copy number of any intact SMN genes determined, given (a) a number of sequence reads of the plurality of sequence reads with bases that support the SMN1 gene-specific base and (b) a number of sequence reads of the plurality of sequence reads with bases that support a SMN2 gene-specific base of the SMN2 gene corresponding to the SMN1 gene-specific base; and
determining a copy number of the SMN1 gene using the most likely combination of the possible copy number of the SMN1 gene and the possible copy number of the SMN2 gene determined for the SMN gene-specific base.

2. The method of claim 1, wherein the sequencing data comprises whole genome sequencing (WGS) data or short-read WGS data.

3.-5. (canceled)

6. The method of claim 1, wherein a sequence read of the plurality of sequence reads is aligned to the first SMN1 or SMN2 region or the second SMN1 or SMN2 region with an alignment quality score of about zero.

7. The method of claim 1, wherein the first SMN1 or SMN2 region comprises the exon 1 to the exon 6 of the SMN1 gene or the SMN2 gene, respectively, and is about 22.2 kb in length, wherein the second SMN1 or SMN2 region comprises the exon 7 and the exon 8 of the SMN gene or the SMN2 gene, respectively, and is about 6 kb in length.

8. The method of claim 1, wherein determining (i) the first normalized number of the sequence reads aligned to the first SMN1 or SMN2 region and (ii) the second normalized number of the sequence reads aligned to the second region comprises: determining (i) the first normalized number of the sequence reads aligned to the first SMN1 or SMN2 region and (ii) the second normalized number of the sequence reads aligned to the second SMN1 or SMN2 region using (i) the length of the first SMN1 or SMN2 region and (ii) the length of the second SMN1 or SMN2 region, respectively, and (iii) a depth of sequence reads of a region of a genome of the subject other than genetic loci comprising the SMN1 gene and the SMN2 gene in the sequence data.

9.-13. (canceled)

14. The method of claim 1, wherein the Gaussian mixture model comprises a one-dimensional Gaussian mixture model.

15. The method of claim 1, wherein the plurality of Gaussians of the Gaussian mixture model represents integer copy numbers 0 to 10.

16. The method of claim 1, wherein a mean of each of the plurality of Gaussians is the integer copy number represented by the Gaussian.

17. The method of claim 1, wherein determining (i) the copy number of the total SMN genes and (ii) the copy number of any intact SMN genes comprises determining (i) the copy number of the total SMN genes and (ii) the copy number of any intact SMN genes using the Gaussian mixture model, and a first predetermined posterior probability threshold, given (i) the first normalized number of the sequence reads aligned to the first SMN1 or SMN2 region and (ii) the second normalized number of the sequence reads aligned to the second SMN1 or SMN2 region, respectively.

18. (canceled)

19. The method of claim 1, comprising determining a copy number of truncated SMN genes using (i) the copy number of the total SMN genes determined and (ii) the copy number of the intact SMN genes determined.

20.-22. (canceled)

23. The method of claim 1, wherein the most likely combination of the possible copy number of the SMN1 gene and the possible copy number of the SMN2 gene is associated with a highest posterior probability, relative to other combinations of the plurality of combinations given (a) the number of sequence reads of the plurality of sequence reads with bases that support the SMN1 gene-specific base and (b) the number of sequence reads of the plurality of sequence reads with bases that support the corresponding SMN2 gene-specific base.

24. The method of claim 1, wherein determining the most likely combination of the possible copy number of the SMN1 gene and the possible combination of the SMN2 gene comprises: determining the most likely combination, of the plurality of possible combinations each comprising a possible copy number of the SMN1 gene and a possible copy number of the SMN2 gene summed to the copy number of any intact SMN genes determined, given a ratio of (a) a number of sequence reads of the plurality of sequence reads with bases that support the SMN1 gene-specific base and (b) a number of sequence reads of the plurality of sequence reads with bases that support the SMN2 gene-specific base of the SMN2 gene corresponding to the SMN gene-specific base.

25. The method of claim 1, wherein determining the most likely combination of the possible copy number of the SMN1 gene and the possible combination of the SMN2 gene comprises:
determining (a) a number of sequence reads of the plurality of sequence reads with bases that support the SMN1 gene-specific base and (b) a number of sequence reads of the plurality of sequence reads with bases that support the SMN2 gene-specific base of the SMN2 gene corresponding to the SMN gene-specific base;
determining the ratio of (a) a number of sequence reads of the plurality of sequence reads with bases that support the SMN1 gene-specific base and (b) a number of sequence reads of the plurality of sequence reads with bases that support the SMN2 gene-specific base of the SMN2 gene corresponding to the SMN gene-specific base; and
determining the most likely combination, of the plurality of possible combinations each comprising a possible copy number of the SMN1 gene and a possible copy number of the SMN2 gene summed to the copy number of any intact SMN genes determined based on the ratio of (a) a number of sequence reads of the plurality of sequence reads with bases that support the SMN1 gene-specific base and (b) a number of sequence reads of the plurality of sequence reads with bases that support the SMN2 gene-specific base of the SMN2 gene corresponding to the SMN1 gene-specific base.

26. The method of claim 1,
wherein determining the most likely combination of the possible copy number of the SMN1 gene and the possible combination of the SMN2 gene comprises: for each of the plurality of SMN1 gene-specific bases, determining a most likely combination, of a plurality of possible combinations each comprising a possible copy number of the SMN1 gene and a possible copy number of the SMN2 gene summed to the copy number of any intact SMN genes determined, associated with a highest posterior probability given (a) a number of sequence reads of the plurality of sequence reads with bases that support the SMN1 gene-specific base and (b) a number of sequence reads of the plurality of sequence reads with bases that support a SMN2 gene-specific base of the SMN2 gene corresponding to the SMN1 gene-specific base, and
wherein determining the copy number of the SMN gene comprises: determining the copy number of the SMN1 gene based on the possible copy number of the SMN1 gene of the most likely combination of the possible copy number of the SMN1 gene and the possible copy number of the SMN2 gene determined for each of the plurality of SMN1 gene-specific bases.

27.-33. (canceled)

34. The method of claim 1, comprising:
receiving race information of the subject; and
selecting the plurality of SMN1 gene-specific bases from pluralities of SMN1 gene-specific bases based on the race information received.

35.-40. (canceled)

41. The method of claim 1, comprising determining a spinal muscular atrophy (SMA) status of the subject based on the copy number of the SMN1 gene.

42. (canceled)

43. The method of claim 1, comprising determining subject is a silent SMA carrier using a number of sequence reads of the plurality of sequence reads aligned to g.27134 of the SMN1 gene and the bases of the sequence reads aligned to the g.27134 of the SMN1 gene.

44. The method of claim 1, comprising determining a treatment recommendation for the subject based on the copy number of the SMN1 gene determined.

45. (canceled)

46. A method for genotyping cytochrome P450 family 2 subfamily D member 6 (CYP2D6) gene comprising:
under control of a hardware processor:
receiving sequence data comprising a plurality of sequence reads obtained from a sample of a subject aligned to cytochrome P450 family 2 subfamily D member 6 (CYP2D6) gene or cytochrome P450 Family 2 Subfamily D Member 7 (CYP2D7) gene;
determining (i) a first number of sequence reads of the plurality of sequence reads aligned to the CYP2D6 gene or the CYP2D7 gene;
determining (i) a first normalized number of the sequence reads aligned to the CYP2D6 gene or the CYP2D7 gene using (i) a length of the CYP2D6 gene or the CYP2D7 gene, respectively;
determining (i) a total copy number of the CYP2D6 gene and the CYP2D7 gene using a Gaussian mixture model comprising a plurality of Gaussians each representing a different integer copy number, given (i) the first normalized number of the sequence reads aligned to the CYP2D6 gene or the CYP2D7 gene;
for one of a plurality of CYP2D6 gene-specific bases, determining a most likely combination, of a plurality of possible combinations each comprising a possible copy number of the CYP2D6 gene and a possible copy number of the CYP2D7 gene summed to the total copy number of the CYP2D6 gene and the CYP2D7 gene determined, given (a) a number of sequence reads of the plurality of sequence reads with bases that support the CYP2D6 gene-specific base and (b) a number of sequence reads of the plurality of sequence reads with bases that support a CYP2D7 gene-specific base of corresponding to the CYP2D6 gene-specific base; and
determining an allele of the CYP2D6 gene the subject has using the most likely combination of the possible copy number of the CYP2D6 gene and the possible copy number of the CYP2D7 gene determined for the CYP2D6 gene-specific base.

47.-96. (canceled)

97. A system for paralog genotyping comprising:
non-transitory memory configured to store executable instructions and sequence data comprising a plurality of sequence reads obtained from a sample of a subject aligned to a first paralog or a second paralog; and
a hardware processor in communication with the non-transitory memory, the hardware processor programmed by the executable instructions to perform:
determining a copy number of paralogs of a first type using a Gaussian mixture model comprising a plurality of Gaussians each representing a different integer copy number given (i) a first number of sequence reads aligned to a first region;
for one of a plurality of first paralog-specific bases, determining a most likely combination, of a plurality of possible combinations each comprising a possible copy number of a first paralog of the first type and a possible copy number of a second paralog of the first type summed to the copy number of the paralogs of the first type determined, given (a) a number of sequence reads of the plurality of sequence reads with bases that support the first paralog-specific base and (b) a number of sequence reads of the plurality of sequence reads with bases that support a second paralog-specific base of the second paralog corresponding to the first paralog-specific base; and
determining a copy number or an allele of first paralog using the most likely combination of the possible copy number of the first paralog and the possible copy number of the second paralog determined for the first paralog-specific base.

98.-106. (canceled)
